(19) United States
(12) Patent Application Publication
(10) Pub. No.: US 2017/0326232 A1
(43) Pub. Date: Nov. 16, 2017

(54) INTRATUMORAL ADMINISTRATION OF PARTICLES CONTAINING A TOLL-LIKE RECEPTOR 9 AGONIST AND A TUMOR ANTIGEN FOR TREATING CANCER

(71) Applicant: Dynavax Technologies Corporation, Berkeley, CA (US)

(72) Inventors: Cristiana GUIDUCCI, Albany, CA (US); Edwina NAIK, Oakland, CA (US); Robert J. MILLEY, Oakland, CA (US); Stewart D. CHIPMAN, Bainbridge Island, WA (US)

(21) Appl. No.: 15/488,353

(22) Filed: Apr. 14, 2017

Related U.S. Application Data
(60) Provisional application No. 62/439,438, filed on Dec. 27, 2016.
(60) Provisional application No. 62/323,622, filed on Apr. 15, 2016.

(51) Int. Cl. A61K 39/395; A61K 31/7088; C12N 15/113; C12Q 1/68

(52) U.S. Cl. CPC A61K 39/395; C12Q 1/6886; A61K 31/7088; C12N 15/1135

(57) ABSTRACT

The present disclosure relates to methods for treating cancer by intratumoral delivery of particles containing a Toll-like receptor 9 agonist (TLR9) and a tumor antigen, in which the TLR9 agonist is a polynucleotide or a chimeric compound thereof. The methods of the present disclosure involve injection of the particles into at least one tumor, and are effective for treating both injected and uninjected tumors of a mammalian subject. Additionally, the present disclosure provides immunogenic compositions containing the particles, as well as methods of manufacture thereof.

### CROSS-REFERENCE TO RELATED APPLICATIONS

This application claims benefit of U.S. Provisional Application No. 62/439,438, filed Dec. 27, 2016, and U.S. Provisional Application No. 62/323,622, filed Apr. 15, 2016, which are hereby incorporated by reference in their entirety.

### SUBMISSION OF SEQUENCE LISTING AS ASCII TEXT FILE

None.

### FIELD

The present disclosure relates to methods for treating cancer by intratumoral delivery of particles containing a Toll-like receptor 9 (TLR9) agonist and a tumor antigen, in which the TLR9 agonist is a polynucleotide or a chimeric compound thereof. The methods of the present disclosure involve injection of the particles into at least one tumor, and are effective for treating both injected and uninjected tumors of a mammalian subject. Additionally, the present disclosure provides immunogenic compositions containing the particles, as well as methods of manufacture thereof.

### BACKGROUND

According to the American Cancer Society, over 500 thousand Americans are expected to die of cancer annually, with cancer accounting for nearly one of every four deaths. In 2015, over 1.5 million new cancer cases are expected to be diagnosed. Although the survival rate has improved over time, over 30% of cancer patients still die within five years of diagnosis.

Polynucleotides containing unmethylated CG dinucleotides stimulate the innate immune system by activating cells expressing Toll-like receptor 9 (TLR9). Several polynucleotide TLR9 agonists have been tested as immunotherapeutic agents for cancer. While results of preclinical and phase II trials of a polynucleotide TLR9 agonist were promising, systemic administration of the polynucleotide TLR9 agonist did not improve survival of patients with non-small cell lung cancer when combined with a chemotherapy regimen (Schmidt, Nature Biotechnology 2007, 25:825-826).

The route of administration of polynucleotide TLR9 agonists has since been shown to be important, with intratumoral injection resulting in superior antitumor immune responses than intravenous injection (Lou et al., J Immunother 2011, 34:279-288). Even so, there remains a need in the art to improve the efficacy of polynucleotide TLR9 agonist-containing cancer vaccines.

### SUMMARY

The present disclosure relates to methods for treating cancer by intratumoral delivery of particles containing a Toll-like receptor 9 agonist (TLR9) and a tumor antigen, in which the TLR9 agonist is a polynucleotide or a chimeric compound thereof. The methods of the present disclosure involve injection of the particles into at least one tumor lesion, and are effective for treating both injected and uninjected tumors in a mammalian subject (e.g., human subject). Additionally, the present disclosure provides immunogenic compositions containing the particles, as well as methods of manufacture thereof.

In particular, the present disclosure provides a method of treating cancer in a mammalian subject (e.g., human subject), the method comprising administering to the subject an effective amount of an immunogenic composition by intratumoral delivery, wherein the immunogenic composition comprises a particle comprising a TLR9 agonist and a tumor antigen each associated with a biocompatible multimerization agent, the multimerization agent has a diameter of 10 to 25,000 nanometers and/or a molecular weight of about 10,000 to about 1,000,000 Daltons, the TLR9 agonist comprises a polynucleotide comprising the sequence 5′-TCGNs-3′ (SEQ ID NO:1), wherein each N is an independently selected nucleoside and s=4 to 47, the tumor antigen comprises a polypeptide, and the TLR9 agonist and the tumor antigen are either each associated with the multimerization agent by one or more covalent linkages, or each associated with the multimerization agent by adsorption. In some embodiments the tumor antigen comprises a polypeptide of about 9 to about 2000 amino acids. In certain preferred embodiments the tumor antigen comprises a polypeptide of about 9 to about 60 amino acids. In some embodiments, the multimerization agent has a diameter of 10 to 25,000 nanometers. In some preferred embodiments the multimerization agent has a diameter of 500 to 5,000 nanometers. In some embodiments, the multimerization agent has a molecular weight of about 10,000 to about 1,000,000 Daltons. In some embodiments, the multimerization agent has a diameter of 10 to 25,000 nanometers and a molecular weight of about 10,000 to about 1,000,000 Daltons. In some preferred embodiments, the multimerization agent has a diameter of 500 to 5,000 nanometers and a molecular weight of about 10,000 to about 1,000,000 Daltons. Unless otherwise noted, both the TLR9 agonist and the tumor antigen are each associated with the same multimerization agent (same complex or molecule).

In some aspects, the present disclosure provides a method of treating cancer in a mammalian subject (e.g., human subject), the method comprising administering to the subject an effective amount of an immunogenic composition by intratumoral delivery, wherein the immunogenic composition comprises a particle comprising a TLR9 agonist and a tumor antigen each associated with a biocompatible multimerization agent, the multimerization agent comprises an aluminum salt complex having a diameter of 0.1 to 25 micrometers, 0.5 to 25 micrometers, or 1 to 25 micrometers, or 0.5 to 5 micrometers, the TLR9 agonist comprises a polynucleotide comprising the sequence 5′-TCGNs-3′ (SEQ ID NO:1), wherein each N is an independently selected nucleoside and s=4 to 47, the tumor antigen comprises a polypeptide, and the TLR9 agonist and the tumor antigen are associated with the same complex by adsorption. In some embodiments, the polypeptide is 8 to 1800 amino acids, about 9 to about 1000 amino acids, or about 10 to about 100 amino acids. Similarly, in some embodiments, the polypeptide is about 9 to about 2000, about 9 to about 1000, about 9 to about 100, or about 9 to about 60 amino acids in length.

In other aspects, the present disclosure provides a method of treating cancer in a mammalian subject (e.g., human subject), the method comprising administering to the subject an effective amount of an immunogenic composition by intratumoral delivery, wherein the immunogenic composition comprises a particle comprising a TLR9 agonist and a tumor antigen each associated with a biocompatible multimerization agent, the multimerization agent comprises a polysaccharide having a diameter of from about 10 to 1,000 nanometers and/or a molecular weight of about 10,000 to about 1,000,000 Daltons, the TLR9 agonist comprises a polynucleotide comprising the sequence 5′-TCGNs-3′ (SEQ ID NO:1), wherein each N is an independently selected nucleoside and s=4 to 47, the tumor antigen comprises a polypeptide, and the TLR9 agonist and the tumor antigen are each associated with the same molecule of the polysaccharide by one or more covalent linkages. In some embodiments, the multimerization agent comprises a polysaccharide having a diameter of from about 10 to 1,000 nanometers. In some embodiments, the multimerization agent comprises a polysaccharide having a molecular weight of about 10,000 to about 1,000,000 Daltons. In some embodiments, the multimerization agent comprises a polysaccharide having a diameter of from about 10 to 1,000 nanometers and a molecular weight of about 10,000 to about 1,000,000 Daltons. In some embodiments, the polypeptide is 8 to 1800 amino acids, about 9 to about 1000 amino acids, or about 10 to about 100 amino acids. Similarly, in some embodiments, the polypeptide is about 9 to about 2000, about 9 to about 1000, about 9 to about 100, or about 9 to about 60 amino acids in length.

In some embodiments, in which the multimerization agent comprises an aluminum salt complex having a diameter of 0.1 to 25 micrometers, about 0.5 to about 25 micrometers, about 1 to about 25 micrometers, or 0.5 to 5 micrometers, and the TLR9 agonist and the tumor antigen are each associated with the same complex by adsorption, the particles are microparticles. In some embodiments, the aluminum salt complex comprises an aluminum hydroxide complex. In other embodiments, in which the multimerization agent comprises a polysaccharide having a diameter of from about 10 to about 1,000 nanometers and/or a molecular weight of about 10,000 to about 1,000,000 Daltons, and the TLR9 agonist and the tumor antigen are each associated with the same molecule of the polysaccharide by one or more covalent linkages, the particles are nanoparticles. In some embodiments, the polysaccharide is selected from the group consisting of a branched copolymer of sucrose and epichlorohydrin, dextran, mannan, chitosan, agarose, and starch. In some embodiments, the polysaccharide is a branched copolymer of sucrose and epichlorohydrin having a molecular weight of about 100,000 to about 700,000 Daltons. In some embodiments, the polysaccharide is a branched copolymer of sucrose and epichlorohydrin having a molecular weight of about 400,000±100,000 Daltons (e.g., FICOLL® PM 400 marketed by GE Healthcare).

In some embodiments, the TLR9 agonist is a polynucleotide consisting of: 5′-(TCG(N_{q}))ᵢN_{w}(X₁X₂CGX₂′X₁′(CG)ₚ)ⱼNᵥ-3′ (SEQ ID NO:2), wherein each N is an independently selected nucleoside; p=0 or 1; q=0, 1, 2, 3, 4 or 5; v=0 to 41; w=0, 1 or 2; i=1, 2, 3 or 4; j=1, 2, 3 or 4; X₁ and X₁′ are self-complementary nucleosides; and X₂ and X₂′ are self-complementary nucleosides; and wherein the polynucleotide is from 9 to 50 nucleotides in length. In some of these embodiments, q=0, 1 or 2. In some of these embodiments, p=0 or 1; q=0, 1 or 2; v=0 to 20; w=0; i=1; and j=1, 2, 3 or 4.

In some embodiments, the TLR9 agonist is a polynucleotide consisting of: 5′-TCGN_{q}(X₁X₂CGX₂′X₁′CG)ⱼNᵥ-3′ (SEQ ID NO:3), wherein each N is an independently selected nucleoside; q=0, 1, 2, 3, 4, or 5; v=1 to 39; j=1, 2, 3 or 4; X₁ and X₁′ are self-complementary nucleosides; and X₂ and X₂′ are self-complementary nucleosides; and wherein the polynucleotide is from 12 to 50 nucleotides in length.

In some embodiments, the TLR9 agonist is a polynucleotide consisting of: 5′-TCGN_{q}AACGTTCGAACGTTCGAANᵣ-3′ (SEQ ID NO:4), wherein each N is an independently selected nucleoside; q=0, 1, 2, 3, 4 or 5; and r=0 to 29.

In some embodiments, the TLR9 agonist is a polynucleotide consisting of a sequence selected from the group consisting of:

(SEQ ID NO: 6)

5′-TCG AAC GTT CGA ACG TTC GAA CGT TCG AAT-3′;

(SEQ ID NO: 7)

5′-TCG TTC GAA CGT TCG AAC GTT CGA A-3′;

(SEQ ID NO: 8)

5′-TCG AAC GTT CGA ACG TTC GAA TTT T-3′;

(SEQ ID NO: 9)

5′-TCG TAA CGT TCG AAC GTT CGA ACG TTA-3′;
and

(SEQ ID NO: 10)

5′-TCG TAA CGT TCG AAC GTT CGA AC-3′.

In some embodiments, the TLR9 agonist is a polynucleotide consisting of 5′-TCG AAC GTT CGA ACG TTC GAA CGT TCG AAT-3′(SEQ ID NO:6).

In some embodiments, the TLR9 agonist is a chimeric compound of the formula Nu1-Sp1-Nu2-Sp2-Nu3, wherein Nu1, Nu2 and Nu3 are independently selected nucleic acid moieties from 7 to 50 nucleotides in length, and Nu1 consists of the sequence 5′-TCGNs-3′ where s=4 to 47, wherein Sp1 and Sp2 are the same or different non nucleic acid spacer moieties comprising at least one member of the group consisting of hexaethylene glycol (HEG), triethylene glycol (TEG), propyl, butyl and hexyl, and wherein Sp1 is covalently linked to Nu1 and Nu2, and Sp2 is covalently linked to Nu2 and Nu3. In some of these embodiments, Nu2 consists of the sequence 5′-AACGTTNm-3′ where m=1 to 44 (SEQ ID NO:73). In some of these embodiments, Nu3 consists of the sequence 5′-AACGTTNm-3′ where m=1 to 44 (SEQ ID NO:73). In some of these embodiments, Nu2 and Nu3 independently consist of the sequence 5′-AACGTTNm-3′ where m=1 to 44 (SEQ ID NO:73). In some of these embodiments, the TLR9 agonist is a chimeric compound comprising three nucleic acid moieties and two hexaethylene glycol (HEG) spacers as

(SEQ ID NO: 5)

5′-TCGGCGC-3′-HEG-5′-AACGTTC-3′-HEG-5′-TCGGCGC-3′
or

(SEQ ID NO: 72)

5′-TCGCCGG-3′-HEG-5′-AACGTTC-3′-HEG-5′-TCGCCGG-3′.

In some embodiments, one or more linkages between nucleotides of the polynucleotide or chimeric compound and/or between the nucleotides and the spacers of the chimeric compound are phosphorothioate ester linkages. In some of these embodiments, all of the linkages between nucleotides and between the nucleotides and the spacers are phosphorothioate ester linkages.

In some embodiments of the method of treating cancer in a mammalian subject (e.g., human subject) comprising administering to the subject an effective amount of an immunogenic composition by intratumoral delivery, wherein the immunogenic composition comprises a particle comprising a TLR9 agonist and a tumor antigen each associated with a biocompatible multimerization agent that comprises a polysaccharide, the composition comprises a heterogeneous mixture of particles in which the average molar ratio of the TLR9 agonist to the polysaccharide and the average molar ratio of the antigen to the polysaccharide are each within the range of from about 10 to about 120.

In some embodiments of the method of treating cancer in a mammalian subject (e.g., human subject) comprises administering to the subject an effective amount of an immunogenic composition by intratumoral delivery, wherein the immunogenic composition comprises a particle comprising a TLR9 agonist and a tumor antigen each associated with a biocompatible multimerization agent that comprises an aluminum salt complex, wherein the composition comprises a heterogeneous mixture of particles in which the ratio of the TLR9 agonist to the aluminum salt complex and the ratio of the antigen to the aluminum salt complex are each within the range of from about 0.1 to about 1 (weight/weight). In some embodiments, the composition comprises a heterogeneous mixture of particles in which the ratio of the TLR9 agonist to the aluminum salt complex is within the range of from about 0.1 to about 1 (weight/weight), while the ratio of the antigen to the aluminum salt complex is with a broader range of from 0.005 to about 1 (weight/weight). In further embodiments, the composition comprises a mixture of particles in which the ratio of the antigen and the TLR9 agonist co-adsorbed to the aluminum salt complex are each within the range of about 0.1 to about 2.5 (w/w), or within the range of about 0.1 to about 5.0 (w/w).

In some embodiments, the tumor antigen comprises the amino acid sequence of a full length protein or a fragment thereof (e.g., a polypeptide of about 10 to about 100 amino acids in length). In some embodiments, the tumor antigen comprises a full length protein or polypeptide fragment of one or more of the group consisting of WT1, MUC1, LMP2, HPV E6, HPV E7, EGFRvIII, Her-2/neu, idiotype, MAGE A3, p53, NY-ESO-1 (CTAG1), PSMA, CEA, MelanA/Mart1, Ras, gp100, proteinase 3, bcr-able, tyrosinase, survivin, PSA, hTERT, sarcoma translocation breakpoints, EphA2, PAP, MP-IAP, AFP, EpCAM, ERG, NA17-A, PAX3, ALK, androgen receptor, cyclin B1, MYCN, PhoC, TRP-2, mesothelin, PSCA, MAGE A1, CYP1B1, PLAC1, BORIS, ETV6-AML, NY-BR-1, RGS5, SART3, carbonic anhydrase IX, PAX5, OY-TESL sperm protein 17, LCK, HMWMAA, AKAP-4, SSX2, XAGE 1, B7-H3, legumain, Tie 2, Page4, VEGFR2, MAD-CT-1, FAP, PAP, PDGFR-beta, MAD-CT-2, CEA, TRP-1 (gp75), BAGE1, BAGE2, BAGE3, BAGE4, BAGE5, CAMEL, MAGE-A2, MAGE-A4, MAGE-A5, MAGE-A6, MAGE-A8, MAGE-A9, MAGE-A10, MAGE-A11, MAGE-A12, and Fos-related antigen 1. In some preferred embodiments, the tumor antigen comprises an amino acid sequence or fragment thereof from one or more of the group consisting of gp100, hTERT, MAGE A1, MAGE A3, MAGE A10, MelanA/Mart1, NY-ESO-1, PSA, Ras, survivin, TRP1 (gp75), TRP2, and tyrosinase.

In some of these embodiments, the tumor antigen is a fusion protein comprising two or more polypeptides, wherein each polypeptide comprises amino acid sequences from different tumor antigens or non-contiguous amino acid sequences from the same tumor antigen. In one variation, the fusion protein comprises a first polypeptide and a second polypeptide, wherein each polypeptide comprises non-contiguous amino acid sequences from the same tumor antigen. In some of these embodiments, the tumor antigen comprises a mammalian antigen expressed by cells of the tumor. In one variation, the mammalian antigen is a neoantigen or encoded by a gene comprising a mutation relative to the gene present in normal cells from the mammalian subject. In some of these embodiments, the tumor antigen comprises a viral antigen expressed by the tumor. In one variation, the viral antigen comprises one or both of HPV E6 and HPV E7. In some preferred embodiments, the tumor antigen comprises the amino acid sequence of a human cancer/testis antigen 1 (CTAG1, also known as NY-ESO-1) protein or fragment thereof. In some variations, the tumor antigen comprises the amino acid sequence of one of the group consisting of SEQ ID NO:52, SEQ ID NO:53, SEQ ID NO:54, SEQ ID NO:55, SEQ ID NO:56, SEQ ID NO:57, SEQ ID NO:58, SEQ ID NO:59, and combinations thereof. In some preferred embodiments described herein, the mammalian subject is a human.

In some embodiments of the method, intratumoral delivery comprises injection of the immunogenic composition into at least one tumor. In some of these embodiments, treating cancer comprises inducing accumulation of tumor antigen-specific T cells in the injected tumor, for example, at greater numbers than had the immunogenic composition been administered at an extratumoral site. In some of these embodiments, treating cancer comprises eliciting a systemic tumor antigen-specific T cell response, for example, a systemic tumor antigen-specific T cell response of a higher magnitude than had the immunogenic composition been administered at an extratumoral site. In some of these embodiments, treating cancer comprises eliciting a systemic tumor antigen-specific T cell response. In some of these embodiments, treating cancer comprises reducing numbers of CD4+FoxP3+ regulatory T cells in the injected tumor. In some of these embodiments, the subject has one or more uninjected tumors in addition to the injected tumor and treating cancer comprises one or more of the following: (a) reducing number of uninjected tumors; (b) reducing volume of uninjected tumors; and (c) retarding growth of uninjected tumors. In some of these embodiments, treating cancer comprises one or more of the following: (d) increasing survival time of the subject; (e) reducing volume of the injected tumor; and (f) retarding growth of the injected tumor. In some embodiments, treating cancer comprises increasing progression free survival or increasing time to progression.

In some embodiments, the tumor is a sarcoma or a carcinoma. In some embodiments, the tumor is a lymphoma. In some embodiments, the cancer is selected from the group consisting of breast cancer, prostate cancer, lung cancer, colorectal cancer, uterine cancer, bladder cancer, melanoma, head and neck cancer, non-Hodgkin lymphoma, kidney cancer, ovarian cancer, pancreatic cancer, and thyroid cancer. In some embodiments, the cancer is a primary cancer of a site selected from the group consisting of oral cavity, digestive system, respiratory system, skin, breast, genital system, urinary system, ocular system, nervous system, endocrine system and lymphoma.

In some embodiments, the method further comprises administering an effective amount of a second therapeutic agent to the subject. In some of these embodiments, the second therapeutic agent comprises a chemotherapeutic agent selected from the group consisting of actinomycin, afatinib, alectinib, asparaginase, azacitidine, azathioprine, bicalutamide, binimetinib, bleomycin, bortezomib, camptothecin, carboplatin, capecitabine, carmustine, certinib, cisplatin, chlorambucil, cobimetinib, crizotinib, cyclophosphamide, cytarabine, dabrafenib, dacarbazine, daunorubicin, docetaxel, doxifluridine, doxorubicin, encorafenib, erlotinib, epirubicin, epothilone, etoposide, fludarabine, flutamine, fluorouracil, gefitinib, gemcitabine, hydroxyurea, idarubicin, ifosfamide, imatinib, irinotecan, lapatinib, letrozole, mechlorethamine, mercaptopurine, methotrexate, mitomycin, mitoxantrone, octreotide, oxaliplatin, paclitaxel, pemetrexed, raltitrexed, sorafenib, sunitinib, tamoxifen, temozolomide, teniposide, tioguanine, topotecan, trametinib, valrubicin, vemurafenib, vinblastine, vincristine, vindesine, vinorelbine, and combinations thereof. In some embodiments, the second therapeutic agent comprises one or both of a BRAF inhibitor and a MEK inhibitor. In some embodiments, the second therapeutic agent comprises a epigenetic modulator selected from the group consisting of HDAC inhibitors (see e.g., voronistat [SAHA], romidepsin, entinostat, abexinostat, elinostat [CHR-3996], panobinostat, quisinostat [JNJ-26481585], 4SC-202, resminostat [SB939], pracinostat [CI-9940], and valproate), and DNA methyltransferase inhibitors (see e.g., azacytidine, decitabine, zebularine, SGI-1027, RG-108, and sinfungin), and combinations thereof.

In some of these embodiments, the second therapeutic agent is an antagonist of an inhibitory immune checkpoint molecule, for example, an inhibitory immune checkpoint molecule selected from the group consisting of PD-1, PD-L1, PD-L2, CTLA-4 (CD152), LAG-3, TIM-3, TIGIT, IL-10, indoleamine 2,3-dioxygenase (IDO), P-selectin glycoprotein ligand-1 (PSGL-1) and TGF-beta. In some of these embodiments, the second therapeutic agent is an agonist of an immune stimulatory molecule. In some of these embodiments, the immune stimulatory molecule is selected from the group consisting of CD27, CD40, OX40 (CD134), GITR, 4-1BB CD137, CD28 and ICOS (CD278). In some of these embodiments, the second therapeutic agent comprises an antibody, fragment or derivative thereof. In some of these embodiments, the second therapeutic agent is an antagonist of an inhibitory immune checkpoint molecule and the second therapeutic agent comprises an antibody, fragment or derivative thereof.

In some embodiments, the method further comprises administering radiation therapy and/or administering an effective amount of a second therapeutic agent to the subject. In some of these embodiments, the effective amount of the immunogenic composition and the effective amount of the second therapeutic agent together result in a cooperative effect or better against the tumor. In some of these embodiments, the effective amount of the immunogenic composition and the effective amount of the second therapeutic agent together result in an additive effect or better against the tumor. In some of these embodiments, the effective amount of the immunogenic composition and the effective amount of the second therapeutic agent together result in a synergistic effect against the tumor.

In some embodiments of the method, treating cancer does not result in development of flu-like symptoms of such severity that repeated administration of the immunogenic composition is contraindicated, wherein the flu-like symptoms comprise one or more of the group consisting of fever, headache, chills, myalgia and fatigue.

In some aspects, the present disclosure provides an immunogenic composition comprising a particle comprising a TLR9 agonist and a tumor antigen each associated with a biocompatible multimerization agent, wherein: the multimerization agent has a diameter of 10 to 10,000 nanometers and/or a molecular weight of about 10,000 to about 1,000,000 Daltons; the TLR9 agonist comprises a polynucleotide comprising the sequence 5′-TCGNs-3′ (SEQ ID NO:1), wherein each N is an independently selected nucleoside, s=4 to 47; the tumor antigen comprises a polypeptide of 8 to 1800 amino acids, about 9 to about 1000 amino acids, or about 10 to about 100 amino acids; and the TLR9 agonist and the tumor antigen are either each associated with the multimerization agent by one or more covalent linkages, or each associated with the multimerization agent by adsorption. In some embodiments, the multimerization agent is an aluminum salt complex, and the TLR9 agonist and the tumor antigen are each associated with the same complex by adsorption. In one variation, the aluminum salt complex comprises an aluminum hydroxide complex. In some embodiments, the multimerization agent is a polysaccharide, and the TLR9 agonist and the tumor antigen are each associated with the same molecule of the polysaccharide by one or more covalent linkages. In one variation, the polysaccharide is selected from the group consisting of a branched copolymer of sucrose and epichlorohydrin, dextran, mannan, chitosan, agarose, and starch. In a further variation, the polysaccharide is a branched copolymer of sucrose and epichlorohydrin having a molecular weight of about 100,000 to about 700,000 Daltons, or about 300,000 to about 500,000 Daltons, or about 400,000±100,000 Daltons (e.g., a FICOLL® PM 400 marketed by GE Healthcare).

In some embodiments of the composition, the particle is a compound of formula (I):

[D-L¹-L²-(PEG)-L³]ₓ-F-[L³-(PEG)-L²-A]ₜ (I),

wherein:
D is the TLR9 agonist;
L¹ is a first linker comprising an alkylthio group;
L² is a second linker comprising a succinimide group;
L³ is a third linker comprising an amide group;
PEG is a polyethylene glycol (e.g., —(OCH₂CH₂)ₙ—, where n is an integer from 2 to 80);
t and x are independently integers from 3 to 200;
A is the tumor antigen; and
F is the polysaccharide, which is connected to L³ via an ether group.

In some embodiments of the composition, the TLR9 agonist is a polynucleotide consisting of 5′-TCGN_{q}AACGTTCGAACGTTCGAANᵣ-3′ (SEQ ID NO:4), wherein each N is an independently selected nucleoside, q=0, 1, 2, 3, 4 or 5, and r=0 to 29. In one variation, the TLR9 agonist is a polynucleotide consisting of 5′-TCG AAC GTT CGA ACG TTC GAA CGT TCG AAT-3′ (SEQ ID NO:6).

In some embodiments of the composition, the TLR9 agonist is a chimeric compound of the formula Nu1-Sp1-Nu2-Sp2-Nu3, wherein Nu1, Nu2 and Nu3 are independently selected nucleic acid moieties from 7 to 50 nucleotides in length, and Nu1 consists of the sequence 5′-TCGNs-3′ where s=4 to 47; wherein Sp1 and Sp2 are the same or different non nucleic acid spacer moieties comprising at least one member of the group consisting of hexaethylene glycol (HEG), triethylene glycol (TEG), propyl, butyl and hexyl; and wherein Sp1 is covalently linked to Nu1 and Nu2, and Sp2 is covalently linked to Nu2 and Nu3. In some of these embodiments, Nu2 and/or Nu3 independently consist of the sequence 5′-AACGTTNm-3′ where m=1 to 44 (SEQ ID NO:73). In some of these embodiments, the TLR9 agonist is a chimeric compound comprising three nucleic acid moieties and two hexaethylene glycol (HEG) spacers as

(SEQ ID NO: 5)

5′-TCGGCGC-3′-HEG-5′-AACGTTC-3′-HEG-5′-TCGGCGC-3′
or

(SEQ ID NO: 72)

5′-TCGCCGG-3′-HEG-5′-AACGTTC-3′-HEG-5′-TCGCCGG-3′.

In some embodiments of the composition where the TLR9 agonist is a chimeric compound of the formula Nu1-Sp1-Nu2-Sp2-Nu3, one or more linkages between nucleotides of the polynucleotide or chimeric compound and/or between the nucleotides and the spacers of the chimeric compound are phosphorothioate ester linkages. In one variation, all of the linkages between nucleotides and between the nucleotides and the spacers are phosphorothioate ester linkages.

In some aspects, provided is a method for preparing a compound of formula (I):

[D-L¹-L²-(PEG)-L³]ₓ-F-[L³-(PEG)-L²-A]ₜ (I),

wherein:
D is a TLR9 agonist;
L¹ is a first linker comprising an alkylthio group;
L² is a second linker comprising a succinimide group;
L³ is a third linker comprising an amide group;
PEG is a polyethylene glycol (e.g., —(OCH₂CH₂)ₙ—, where n is an integer from 2 to 80);
t and x are independently an integer from 3 to 200;
A is a tumor antigen comprising a polypeptide of about 9 to about 1000 amino acids; and
F is a polysaccharide having a molecular weight of about 10,000 to about 1,000,000 Daltons and is connected to L³ via an ether group,
wherein the TLR9 agonist comprises a polynucleotide comprising the sequence 5′-TCGNs-3′, wherein s=4 to 47 and each N is a nucleoside, and wherein one or more linkages between the nucleotides and between the 3′-terminal nucleotide and L¹ are phosphorothioate ester linkages, and
wherein A is a tumor antigen comprising a polypeptide of about 9 to about 1000 amino acids and comprises at least one thiol group,
the method comprising:
reacting a compound of the formula D-L^{1a}-SH, where D is as defined for formula (I) and L^{1a} is (CH₂)ₘ where m is an integer from 2 to 9, and reacting a compound of the formula A, with a compound of formula (II):

[L^{2a}-(PEG)-L³]_{y}-F (II)
wherein L³, PEG and F are as defined for formula (I);
L^{2a} is

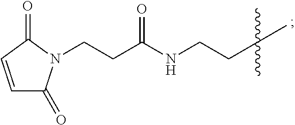 [SMILES: *CCNC(=O)CCN1C(=O)C=CC1=O]

and
y is an integer from 3 to 350; provided that y is no less than the sum of t and x.

In some embodiments of the method for preparing a compound of formula (I), the method comprises reacting a compound of the formula D-L^{1a}-SH with a compound of formula (II) for form an intermediate, and subsequently reacting a compound of the formula A with the intermediate. In some embodiments, the method comprises simultaneously reacting a compound of the formula D-L^{1a}-SH and a compound of the formula A with a compound of formula (II). In some of these embodiments, the reaction is carried out in a medium comprising guanidine hydrochloride.

In some embodiments of the method for preparing a compound of formula (I), D is a chimeric compound of the formula Nu1-Sp1-Nu2-Sp2-Nu3, wherein Nu1, Nu2 and Nu3 are independently selected nucleic acid moieties from 7 to 50 nucleotides in length, and Nu1 consists of the sequence 5′-TCGNs-3′ where s=4 to 47; wherein Sp1 and Sp2 are the same or different non nucleic acid spacer moieties comprising at least one member of the group consisting of hexaethylene glycol (HEG), triethylene glycol (TEG), propyl, butyl and hexyl; and wherein Sp1 is covalently linked to Nu1 and Nu2, and Sp2 is covalently linked to Nu2 and Nu3. In some of these embodiments, Nu2 consists of the sequence 5′-AACGTTNm-3′ where m=1 to 44 (SEQ ID NO:73). In some of these embodiments, Nu3 consists of the sequence 5′-AACGTTNm-3′ where m=1 to 44 (SEQ ID NO:73). In some of these embodiments, Nu2 and Nu3 independently consist of the sequence 5′-AACGTTNm-3′ where m=1 to 44 (SEQ ID NO:73). In some of these embodiments, D is

(SEQ ID NO: 5)

5′-TCGGCGC-3′-HEG-5′-AACGTTC-3′-HEG-5′-TCGGCGC-3′
or

(SEQ ID NO: 72)

5′-TCGCCGG-3′-HEG-5′-AACGTTC-3′-HEG-5′-TCGCCGG-3′.

In some embodiments of the method for preparing a compound of formula (I), the polypeptide comprises at least one cysteine residue. In some of these embodiments, the at least one cysteine residue is located at the N-terminus or the C-terminus of the polypeptide, or is within five amino acids of the N-terminus or the C-terminus of the polypeptide.

In some embodiments of the method for preparing a compound of formula (I), the polysaccharide is selected from the group consisting of a branched copolymer of sucrose and epichlorohydrin, a dextran, a mannan, a chitosan, an agarose, and a starch. In some embodiments, he polysaccharide is a branched copolymer of sucrose and epichlorohydrin having a molecular weight of about 100,000 to about 700,000 Daltons, or about 300,000 to about 500,000 Daltons, or about 400,000±100,000 Daltons (e.g., a FICOLL® PM 400 marketed by GE Healthcare).

In some aspects, provided is a method for preparing a co-adsorbate particle comprising a TLR9 agonist and a tumor antigen each associated with a biocompatible multimerization agent by adsorption, wherein:
the multimerization agent is an aluminum salt complex having a diameter of 0.1 to 25 micrometers, about 0.5 to about 25 micrometers, about 1 to about 25 micrometers, or about 0.5 to about 5 micrometers,
the TLR9 agonist comprises a polynucleotide comprising the sequence 5′-TCGNs-3′ (SEQ ID NO:1), wherein s=4 to 47 and each N is a nucleoside, and
the tumor antigen comprises a polypeptide of 8 to 1800 amino acids, about 9 to about 1000 amino acids, or about 10 to about 100 amino acids,
the method comprising:
adding the tumor antigen dissolved in an aqueous solution containing about 5% to about 30% isopropanol, and adding the TLR9 agonist, to the aluminum salt complex equilibrated in a buffer,
wherein the buffer is in a pH range of about 6 to about 9 and the buffer is not a phosphate buffer.

In some embodiments of the method for preparing the co-adsorbate particle, the aluminum salt complex comprises an aluminum hydroxide complex. In some embodiments, the buffer is in a pH range of about 7 to about 8. In some embodiments, the tumor antigen is dissolved in an aqueous solution containing about 10% to about 20% of an organic solvent (e.g., isopropanol). In some embodiments, the TLR9 agonist is dissolved in an acetate buffer having a pH of about 7. In some embodiments, the tumor antigen and the TLR9 agonist are adsorbed to the aluminum salt complex at the same time. In some embodiments, the tumor antigen is adsorbed to the aluminum salt complex first followed by adsorption of the TLR9 agonist. In some embodiments, the TLR9 agonist is adsorbed to the aluminum salt complex first followed by adsorption of the tumor antigen. In some embodiments, the TLR9 agonist is a polynucleotide consisting of 5′-TCGN_{q}AACGTTCGAACGTTCGAANᵣ-3′ (SEQ ID NO:4), wherein each N is an independently selected nucleoside, q=0, 1, 2, 3, 4 or 5, and r=0 to 29. In one variation, the TLR9 agonist is a polynucleotide consisting of 5′-TCG AAC GTT CGA ACG TTC GAA CGT TCG AAT-3′ (SEQ ID NO:6). In some embodiments, the TLR9 agonist is a polynucleotide consisting of a polynucleotide sequence selected from group consisting of SEQ ID NO:2, SEQ ID NO:3, SEQ ID NO:7, SEQ ID NO:8, SEQ ID NO:9, and SEQ ID NO:10. In some embodiments, the TLR9 agonist is a chimeric compound of the formula Nu1-Sp1-Nu2-Sp2-Nu3, wherein Nu1, Nu2 and Nu3 are independently selected nucleic acid moieties from 7 to 50 nucleotides in length, and Nu1 consists of the sequence 5′-TCGNs-3′ where s=4 to 47; wherein Sp1 and Sp2 are the same or different non nucleic acid spacer moieties comprising at least one member of the group consisting of hexaethylene glycol (HEG), triethylene glycol (TEG), propyl, butyl and hexyl; and wherein Sp1 is covalently linked to Nu1 and Nu2, and Sp2 is covalently linked to Nu2 and Nu3. In some of these embodiments, Nu2 and/or Nu3 independently consist of the sequence 5′-AACGTTNm-3′ where m=1 to 44 (SEQ ID NO:73). In some of these embodiments, the TLR9 agonist is

(SEQ ID NO: 5)

5′-TCGGCGC-3′-HEG-5′-AACGTTC-3′-HEG-5′-TCGGCGC-3′
or

(SEQ ID NO: 72)

5′-TCGCCGG-3′-HEG-5′-AACGTTC-3′-HEG-5′-TCGCCGG-3′.

In some preferred embodiments of the present disclosure, the tumor antigen comprises a polypeptide of from 8 to 1800 amino acids in length, preferably 9 to 1000 amino acids in length, and more preferably from 10 to 100 amino acids in length. In some variations, the tumor antigen is a fusion protein comprising two or more polypeptides, wherein each polypeptide comprises amino acid sequences from different tumor antigens or non-contiguous amino acid sequences from the same tumor antigen. In some variations, the fusion protein comprises a first polypeptide and a second polypeptide, wherein each polypeptide comprises non-contiguous amino acid sequences from the same tumor antigen. In some variations, the tumor antigen comprises a neoantigen encoded by a gene comprising a mutation relative to the gene present in normal cells from the mammalian subject. In other variations, the tumor antigen comprises a viral antigen expressed by the tumor. In some preferred embodiments, the tumor antigen comprises the amino acid sequence of a human cancer/testis antigen 1 (CTAG1 also known as NY-ESO-1) protein or a fragment thereof. In some variations, the tumor antigen comprises the amino acid sequence of one of the group consisting of SEQ ID NO:52, SEQ ID NO:53, SEQ ID NO:54, SEQ ID NO:55, SEQ ID NO:56, SEQ ID NO:57, SEQ ID NO:58, SEQ ID NO:59, and combinations thereof.

The present disclosure provides in some preferred embodiments, an immunogenic composition comprising a particle comprising a TLR9 agonist and a tumor antigen each associated with an aluminum hydroxide complex; wherein the aluminum hydroxide complex has a diameter of about 0.1 to about 25 micrometers (preferably about 0.5 to about 25 micrometers or about 0.5 to about 2.5 micrometers); the TLR9 agonist comprises a polynucleotide comprising the sequence of SEQ ID NO:6; the tumor antigen comprises a polypeptide having the amino acid sequence of the human cancer/testis antigen 1 of SEQ ID NO:60 or a fragment thereof that is at least eight amino acids in length; and the TLR9 agonist and the tumor antigen are either each associated with the aluminum hydroxide complex by adsorption. In some embodiments, the tumor antigen comprises the amino acid sequence of one of the group consisting of SEQ ID NO:52, SEQ ID NO:53, SEQ ID NO:54, SEQ ID NO:55, SEQ ID NO:56, SEQ ID NO:57, SEQ ID NO:58, SEQ ID NO:59, and combinations thereof. In some embodiments, the composition comprises a heterogeneous mixture of particles in which the mean ratio of the TLR9 agonist to the aluminum salt complex is within a range of about 0.2 to about 1.2 (weight/weight), the mean ratio of the tumor antigen to the aluminum salt complex is within a range of from about 0.005 to about 2.0 (weight/weight) and the weight of the aluminum salt complex is based on aluminum content. Also provided are methods of treating cancer in a mammalian subject (e.g., a human subject), comprising administering to the subject an effective amount of the immunogenic composition by intratumoral delivery.

Moreover, the present disclosure provides a method of preparing a sterile immunogenic composition, comprising the steps of:
(a) dissolving one or more peptide antigens in an aqueous solution comprising an organic solvent to produce an aqueous peptide solution;
(b) contacting the aqueous peptide solution with a slurry comprising an aluminum hydroxide complex to produce particles comprising peptide antigens adsorbed to the aluminum hydroxide complex;
(c) isolating the peptide-aluminum hydroxide particles and reconstitution in a neutral buffer to produce a buffered peptide-aluminum hydroxide particle solution;
(d) autoclaving the buffered peptide-aluminum hydroxide particle solution to produce a sterile particle solution;
(e) dissolving a TLR9 agonist in a neutral buffer to produce a buffered TLR9 agonist solution;
(f) passing the buffered TLR9 agonist solution through an about 0.2 micrometer filter to produce a sterile TLR9 agonist solution; and
(g) contacting the sterile particle solution and the sterile TLR9 agonist solution to produce a sterile immunogenic solution comprising particles comprising the TLR9 agonist and the peptide antigens each adsorbed to the aluminum hydroxide complex; wherein:
the one or more peptide antigens are tumor antigens each comprising a polypeptide of about 9 to 2000 amino acids in length,
the aluminum hydroxide complex has a diameter of about 500 to about 5,000 nanometers, and
the TLR9 agonist comprises a CpG-containing polynucleotide of 12 to 50 nucleotides in length. In some embodiments, the organic solvent is selected from the group consisting of isopropyl alcohol, dimethyl sulfoxide, dimethyformamide, formic acid, ethanol, 2-butanol, acetone, acetic acid, and combinations thereof. In some embodiments, the tumor antigens each comprise a polypeptide of about 8 to about 60 amino acids in length. In some embodiments, the neutral buffer is in a pH range of about 6 to about 9 and the buffer is not a phosphate buffer. In some embodiments, steps (a)-(d) occur before or concurrently with steps (e) and (f). In some embodiments, the sterile immunogenic composition comprises a heterogeneous mixture of particles in which the ratio of each of the peptide antigens to the aluminum hydroxide complex and the ratio of the TLR9 agonist to the aluminum hydroxide complex are within the range of about 0.1 to about 5.0 (weight/weight)(e.g., peptide:alum:TLR9=0.1-5.0:1:0:0.1-5.0). In some embodiments, the TLR9 agonist comprises the sequence 5′-TCGNs-3′ (SEQ ID NO:1), wherein s=4 to 47 and each N is a nucleoside. In a subset of these embodiments, the TLR9 agonist is a polynucleotide consisting of 5′-TCGNqAACGTTCGAACGTTCGAANr-3′ (SEQ ID NO:4), wherein each N is an independently selected nucleoside, q=0, 1, 2, 3, 4 or 5, and r=0 to 29. In some preferred embodiments, the TLR9 agonist is a polynucleotide consisting of 5′-TCG AAC GTT CGA ACG TTC GAA CGT TCG AAT-3′ (SEQ ID NO:6). In some embodiments, the sterile immunogenic composition comprises a heterogeneous mixture of particles in which the ratio of each of the peptide antigens to the aluminum hydroxide complex is in the range of about 0.6 to 1.2:1.0 (w/w), and the ratio of the TLR9 agonist to the aluminum hydroxide complex is in the range of about 1.7 to 3.4:1.0 (w/w). In some preferred embodiments, the ratio of each of the peptide antigens to the aluminum hydroxide complex is about 1.2:1.0 (w/w), and the ratio of the TLR9 agonist to the aluminum hydroxide complex is about 3.4:1.0 (w/w). As described herein, the weight of the aluminum hydroxide complex is based on aluminum content. In other embodiments, the TLR9 agonist is a chimeric compound of the formula Nu1-Sp1-Nu2-Sp2-Nu3, wherein Nu1, Nu2 and Nu3 are independently selected nucleic acid moieties from 7 to 50 nucleotides in length, and Nu1 consists of the sequence 5′-TCGNs-3′ where s=4 to 47, wherein Sp1 and Sp2 are the same or different non nucleic acid spacer moieties comprising at least one member of the group consisting of hexaethylene glycol (HEG), triethylene glycol (TEG), propyl, butyl and hexyl, and wherein Sp1 is covalently linked to Nu1 and Nu2, and Sp2 is covalently linked to Nu2 and Nu3. In some preferred embodiments, the TLR9 agonist is a chimeric compound comprising three nucleic acid moieties and two hexaethylene glycol (HEG) spacers as

(SEQ ID NO: 5)

5′-TCGGCGC-3′-HEG-5′-AACGTTC-3′-HEG-5′-TCGGCGC-3′
or

(SEQ ID NO: 72)

5′-TCGCCGG-3′-HEG-5′-AACGTTC-3′-HEG-5′-TCGCCGG-3′.

In some embodiments, the peptide antigens are tumor antigens. In some preferred embodiments, at least one of the tumor antigens comprises a polypeptide having the amino acid sequence of the human cancer/testis antigen 1 of SEQ ID NO:60 or a fragment thereof that is at least eight amino acids in length. In some embodiments, at least one of the tumor antigens comprises the amino acid sequence of one of the group consisting of SEQ ID NO:52, SEQ ID NO:53, SEQ ID NO:54, SEQ ID NO:55, SEQ ID NO:56, SEQ ID NO:57, SEQ ID NO:58, SEQ ID NO:59, and combinations thereof. Also provided are methods of treating cancer in a mammalian subject (e.g., a human subject), comprising administering to the subject an effective amount of the immunogenic composition by intratumoral delivery.

### BRIEF DESCRIPTION OF DRAWINGS

FIG. 1A-B provides a flow chart for the manufacturing scheme used to prepare an exemplary particle (nanoparticle) comprising a TLR9 agonist (CpG) and a tumor antigen (peptide), each conjugated to a polysaccharide multimerization agent (FICOLL® brand polysaccharide marketed by GE Healthcare), as [CpG-PEG₆]ₓ-FICOLL-[(PEG₆-peptide]ₜ.

FIG. 2 illustrates preparation of an exemplary particle (nanoparticle) comprising a TLR9 agonist (CpG) and a tumor antigen (peptide), each conjugated to a polysaccharide multimerization agent (FICOLL), as [CpG-PEG₆]ₓ-FICOLL-[(PEG₆-peptide]ₜ.

FIG. 3A-D provides growth curves depicting the change in tumor volume over time of tumor-bearing mice following intratumoral (IT) or subcutaneous (SC) administration of TLR9 agonist-containing nanoparticles, as compared to unvaccinated controls. Mean tumor volume shown is representative of two independent experiments of groups of 5-6 mice. In FIG. 3A, mice with EG7-OVA lymphoma tumors were left untreated or received TLR9 agonist-containing nanoparticles on days 4, 7 and 11 post-transplant. In two groups, the nanoparticles also contained ovalbumin (OVA) protein. In FIG. 3B, mice with B16-OVA melanoma tumors were left untreated or received TLR9 agonist-containing nanoparticles on days 10, 14 and 17 post-transplant. In two groups, the nanoparticles also contained ovalbumin (OVA) protein. In FIG. 3C, mice with B16-OVA melanoma tumors were left untreated or received TLR9 agonist-containing nanoparticles on days 8, 12 and 16 post-transplant. In two groups, the nanoparticles also contained an ovalbumin polypeptide (OVApep). In FIG. 3D, mice with B16-F10 melanoma tumors were left untreated or received TLR9 agonist-containing nanoparticles on days 8, 12 and 16 post-transplant. In two groups, the nanoparticles also contained a polypeptide including epitopes of three melanoma differentiation antigens (Triple).

FIG. 4 provides a growth curve depicting the change in tumor volume over time of tumor-bearing mice following intratumoral (IT) administration of TLR9 agonist-containing nanoparticles, as compared to unvaccinated controls. Mice with EG7-OVA lymphoma tumors were left untreated or received TLR9 agonist-containing nanoparticles on days 0, 3 and 7. Data representative of two independent experiments are shown as the mean tumor volume of groups of 4-6 mice. In one group the nanoparticles also contained an ovalbumin polypeptide (OVApep), while in another group separate nanoparticles contained OVApep. In the schematic, grey circles depict FICOLL® brand polysaccharide marketed by GE Healthcare, dark wavy lines depict the D61-01 TLR9 agonist (CpG), and dark ovals depict the OVApep antigen. Statistical significance was calculated using unpaired Student's t-test and GraphPad Prism software, with a p value of less than 0.05 considered to be significant.

FIG. 5 provides graphs showing antigen-specific IFN-γ secretion by lymphocytes of tumor-bearing mice following intratumoral (IT) or subcutaneous (SC) administration of TLR9 agonist-containing nanoparticles, as compared to unvaccinated controls. Mice bearing established EG7-OVA lymphoma or B16-OVA melanoma tumors were left untreated or received TLR9 agonist-containing nanoparticles on days 0, 7 and 10. In two groups, the nanoparticles also contained an ovalbumin polypeptide (OVApep). Lymphocytes were obtained from tumor-draining lymph nodes collected on day 13, and restimulated with varying concentrations of the OVA class I peptide. IFN-γ secretion in supernatants was assessed by ELISA. Data representative of two independent experiments is shown as the mean IFN-γ concentration of lymphocytes isolated from lymph nodes pooled from 2-5 mice to generate 2-3 replicates per group.

FIG. 6A provides graphs showing antigen-specific IFN-γ secretion by splenocytes of tumor-bearing mice following intratumoral (IT) or subcutaneous (SC) administration of TLR9 agonist-containing nanoparticles, as compared to unvaccinated controls. Mice bearing established EG7-OVA lymphoma or B16-OVA melanoma tumors were left untreated or received TLR9 agonist-containing nanoparticles on days 0, 3 and 7. In two groups, the nanoparticles also contained an ovalbumin polypeptide (OVApep). Splenocytes were obtained from spleens collected on day 10, and restimulated with varying concentrations of the OVA class I peptide. IFN-γ secretion in supernatants was assessed by ELISA. Data representative of two independent experiments is shown as the mean IFN-γ concentration of lymphocytes isolated from spleens pooled from 2-5 mice to generate 2-3 replicates per group. FIG. 6B provides a schematic of the schedule for establishment of bilateral B16-OVA melanoma tumors and subsequent treatment with TLR9 agonist-containing nanoparticles. FIG. 6C provides growth curves depicting the change in vaccinated and unvaccinated tumor volumes over time of tumor-bearing mice following intratumoral (IT) or subcutaneous (SC) administration of TLR9 agonist-containing nanoparticles, as compared to unvaccinated controls. In two groups, the nanoparticles also contained an ovalbumin polypeptide (OVApep).

FIG. 7A provides a schematic of the schedule for establishment of bilateral B16-OVA melanoma tumors and subsequent treatment with TLR9 agonist-containing nanoparticles (D61-01-Fic-OVApep). Mice were vaccinated IT in the right tumor or at distant site from both tumors (SC) at days 10, 13 and 17 post tumor cell inoculation. Three days after the last immunization, tumors were collected to extract RNA and perform gene expression analysis, and volumes of the left tumors were recorded. FIG. 7B shows that administration of an immunogenic composition (D61-01-Fic-OVApep) by the intratumoral route elicited a stronger anti-tumor response against distant site uninjected tumors as compared to extratumoral administration of the immunogenic composition via subcutaneous injection. FIG. 7C provides graphs depicting the magnitude of defined immune cells types present in the tumor microenvironment, as determined by Nanostring gene expression analysis. Signatures identifying the presence of CD8+ T cells, cytotoxic cells, Th1 cells and NK cells, are significantly upregulated in uninjected tumors from mice vaccinated IT versus mice vaccinated SC or with adjuvant alone (D61-01-Fic). Data represent n=3 per treatment condition. Statistical significance was calculated using unpaired Student's t-test and GraphPad Prism software with values less than 0.05 considered to be significant. *p≦0.05, **p≦0.01 and ***p≦0.001.

FIG. 8A provides a cartoon showing the establishment of B16-OVA melanoma tumors in both the subcutaneous space and in the lung of mice. Mice harboring concomitant subcutaneous tumors and lung tumors were vaccinated with D61-01-Fic-OVApep in the subcutaneously growing tumors (IT) or at distant site (SC). D61-01-Fic adjuvant alone was administered IT as a control. Lung tumors were established by injecting B16-OVA tumor cells by the intravenous route. Mice were vaccinated at days 8, 12, 15 and 18 after the implantation of the subcutaneous tumor. Seven days after last vaccination, mice were sacrificed and lungs were collected. Lungs were then fixed in formalin and numbers of macroscopic metastasis were enumerated. FIG. 8B provides a graph showing volumes of injected tumors, which demonstrates that administering the vaccine directly into the tumor results in superior antitumor activity as compared to distant site immunization (SC) or adjuvant alone. FIG. 8C depicts representative photographs of lungs of mice from each study group. FIG. 8D provides a graph of cumulative metastasis data from two independent experiments. Statistical significance was calculated using unpaired Student's t-test and GraphPad Prism software with values less than 0.05 considered to be significant. *p≦0.05, **p≦0.01 and ***p≦0.001.

FIG. 9 provides a flow chart for the manufacturing scheme used to prepare an exemplary particle (microparticle) comprising a TLR9 agonist (CpG) and one or more tumor antigens (peptides), each co-adsorbed to an aluminum hydroxide particle.

FIG. 10A-C provides growth curves depicting the change in tumor volume over time of tumor-bearing mice following intratumoral (IT) or subcutaneous (SC) administration of TLR9 agonist-containing microparticles, as compared to unvaccinated controls. Mean tumor volume shown is representative of at least two independent experiments of groups of 5-7 mice. In FIG. 10A, mice with B16-OVA melanoma tumors were left untreated or received TLR9 agonist-containing microparticles on days 8, 11 and 15 post-transplant. In two groups, the microparticles also contained an ovalbumin polypeptide (OVApep). In FIG. 10B, mice with B16-OVA melanoma tumors were left untreated or received TLR9 agonist-containing microparticles on days 8, 11 and 15 post-transplant. In two groups, the microparticles also contained a polypeptide including epitopes of three melanoma differentiation antigens (Triple). In FIG. 10C, mice with EG7-OVA lymphoma tumors were left untreated or received TLR9 agonist-containing microparticles on days 8, 11 and 15 post-transplant. In two groups, the microparticles also contained an ovalbumin polypeptide (OVApep). In this experiment, Alum is ALHYDROGEL® 85, an aluminum hydroxide complex marketed by Brenntag Biosector A/S.

FIG. 11A-B demonstrates that administration of immunogenic compositions (DV61-04-Alum-OVApep) by the intratumoral route elicited a superior anti-tumor response as compared to extratumoral administration via subcutaneous injection into a site distant from the tumor. Mice harboring concomitant subcutaneous and lung tumors were vaccinated with DV61-04-Alum-OVApep in a subcutaneously growing tumor (IT vaccine) or at a distant site (SC vaccine). DV61-04-Alum adjuvant alone, given IT, was used as a control. Lung tumors were established by injecting B16-OVA tumor cells by the intravenous route. Mice were vaccinated at days 11, 14, 18 and 21 after the implantation of the subcutaneous tumor. Four days after the last vaccination, mice were sacrificed and lungs were collected. Lungs were then fixed in formalin and numbers of macroscopic metastasis were enumerated. In this experiment, Alum is ALHYDROGEL® 85, an aluminum hydroxide complex marketed by Brenntag Biosector A/S.

### DETAILED DESCRIPTION

The present disclosure relates to methods for treating cancer by intratumoral delivery of particles containing a Toll-like receptor 9 agonist (TLR9) and a tumor antigen, in which the TLR9 agonist is a polynucleotide or a chimeric compound thereof. The methods of the present disclosure involve injection of the particles into at least one tumor, and are effective for treating both injected and uninjected tumors of a mammalian subject. Additionally, the present disclosure provides immunogenic compositions containing the particles, as well as methods of manufacture thereof.

### I. General Methods and Definitions

The practice of the present disclosure will employ, unless otherwise indicated, conventional techniques of molecular biology, microbiology, cell biology, biochemistry, nucleic acid chemistry, and immunology, which are within the skill of the art. Such techniques are fully described in the literature, see for example: Animal Cell Culture, sixth edition (Freshney, Wiley-Blackwell, 2010); Antibodies, A Laboratory Manual, second edition (Greenfield, ed., Cold Spring Harbor Publications, 2013); Bioconjugate Techniques, third edition (Hermanson, Academic Press, 1996); Current Protocols in Cell Biology (Bonifacino et al., ed., John Wiley & Sons, Inc., 1996, including supplements through 2014); Current Protocols in Immunology (Coligan et al., eds., John Wiley & Sons, Inc., 1991 including supplements through 2014); Current Protocols in Molecular Biology (Ausubel et al., eds., John Wiley & Sons, Inc., 1987, including supplements through 2014); Current Protocols in Nucleic Acid Chemistry (Egli et al., ed., John Wiley & Sons, Inc., 2000, including supplements through 2014); Molecular Cloning: A Laboratory Manual, third edition (Sambrook and Russell, Cold Spring Harbor Laboratory Press, 2001); Molecular Cloning: A Laboratory Manual, fourth edition (Green and Sambrook, Cold Spring Harbor Laboratory Press, 2012); Oligonucleotide Synthesis: Methods and Applications (Herdewijn, ed., Humana Press, 2004); Protocols for Oligonucleotides and Analogs, Synthesis and Properties (Agrawal, ed., Humana Press, 1993); and Using Antibodies: A Laboratory Manual (Harlow and Lane, Cold Spring Harbor Laboratory Press, 1998).

As used herein and in the appended claims, the singular forms “a,” “an,” and “the” include plural references unless indicated otherwise. For example, “an” excipient includes one or more excipients.

The phrase “comprising” as used herein is open-ended, indicating that such embodiments may include additional elements. In contrast, the phrase “consisting of” is closed, indicating that such embodiments do not include additional elements (except for trace impurities). The phrase “consisting essentially of” is partially closed, indicating that such embodiments may further comprise elements that do not materially change the basic characteristics of such embodiments. It is understood that aspects and embodiments described herein as “comprising” include “consisting of” and “consisting essentially of” embodiments.

The term “about” as used herein in reference to a value, encompasses from 90% to 110% of that value (e.g., about 20 μg survivin antigen refers to 18 μg to 22 μg survivin antigen and includes 20 μg survivin antigen).

As used interchangeably herein, the terms “polynucleotide,” “oligonucleotide” and “nucleic acid” include single-stranded DNA (ssDNA), double-stranded DNA (dsDNA), single-stranded RNA (ssRNA) and double-stranded RNA (dsRNA), modified oligonucleotides and oligonucleosides, or combinations thereof. Polynucleotides are polymers of nucleosides joined, generally, through phosphodiester linkages, although alternate linkages, such as phosphorothioate esters may also be used. A nucleoside consists of a purine (adenine (A) or guanine (G) or derivative thereof) or pyrimidine (thymine (T), cytosine (C) or uracil (U), or derivative thereof) base bonded to a sugar. The four nucleoside units (or bases) in DNA are called deoxyadenosine, deoxyguanosine, thymidine, and deoxycytidine. The four nucleoside units (or bases) in RNA are called adenosine, guanosine, uridine and cytidine. A nucleotide is a phosphate ester of a nucleoside.

The term “palindromic sequence” or “palindrome” refers to a nucleic acid sequence that is an inverted repeat, e.g., ABCDD′C′B′A′, where the bases, e.g., A, and A′, B and B′, C and C′, D and D′, are capable of forming Watson-Crick base pairs. Such sequences may be single-stranded or may form double-stranded structures or may form hairpin loop structures under some conditions. For example, as used herein, “an 8 base palindrome” refers to a nucleic acid sequence in which the palindromic sequence is 8 bases in length, such as ABCDD′C′B′A′. A palindromic sequence may be part of a polynucleotide that also contains non-palindromic sequences. A polynucleotide may contain one or more palindromic sequence portions and one or more non-palindromic sequence portions. Alternatively, a polynucleotide sequence may be entirely palindromic. In a polynucleotide with more than one palindromic sequence portions, the palindromic sequence portions may or may not overlap with each other.

The terms “individual” and “subject” refer to mammals. “Mammals” include, but are not limited to, humans, non-human primates (e.g., monkeys), farm animals, sport animals, rodents (e.g., mice and rats) and pets (e.g., dogs and cats).

The term “antigen” refers to a substance that is recognized and bound specifically by an antibody or by a T cell antigen receptor. Antigens can include peptides, polypeptides, proteins, glycoproteins, polysaccharides, complex carbohydrates, sugars, gangliosides, lipids and phospholipids; portions thereof and combinations thereof. Antigens when present in the compositions of the present disclosure can be synthetic or isolated from nature. Antigens suitable for administration in the methods of the present disclosure include any molecule capable of eliciting an antigen-specific B cell or T cell response. Haptens are included within the scope of “antigen.” A “hapten” is a low molecular weight compound that is not immunogenic by itself but is rendered immunogenic when conjugated with a generally larger immunogenic molecule (carrier).

“Polypeptide antigens” can include purified native peptides, synthetic peptides, recombinant peptides, crude peptide extracts, or peptides in a partially purified or unpurified active state (such as peptides that are part of attenuated or inactivated viruses, microorganisms or cells), or fragments of such peptides. Polypeptide antigens are preferably at least six amino acid residues in length, preferably from 8 to 1800 amino acids in length, more preferably from 9 to 1000 amino acids in length, or from 10 to 100 amino acids in length. Similarly, in some embodiments, the polypeptide is about 9 to about 2000, about 9 to about 1000, about 9 to about 100, or about 9 to about 60 amino acids in length. In some embodiments, the polypeptide is at least (lower limit) 9, 10, 11, 12, 13, 14, 15, 16, 17, 18, 19, 20, 25, 30, 35, 40, 45, 50, 60, 70, 80 or 90 amino acids in length. In some embodiments, the polypeptide is at most (upper limit) 1000, 900, 800, 700, 600, 500, 400, 300, 250, 200, 150 or 100 amino acids in length. In some embodiments, the polypeptide antigen is from 10 to 100 amino acids in length.

As used herein, the term “immunogenic” refers to the ability of an agent (e.g., polypeptide antigen) to elicit an adaptive immune response upon administration under suitable conditions to a mammalian subject. The immune response may be B cell (humoral) and/or T cell (cellular) response.

“Adjuvant” refers to a substance which, when mixed with an immunogenic agent such as antigen, nonspecifically enhances or potentiates an immune response to the agent in the recipient upon exposure to the mixture.

The term “agonist” is used in the broadest sense and includes any molecule that activates signaling through a receptor. In some embodiments, the agonist binds to the receptor. For instance, a TLR9 agonist binds to a TLR9 receptor and activates a TLR9-signaling pathway. In another example, an agonist of the immune stimulatory molecule CD27 binds to and activates a CD27 signaling pathway.

The term “antagonist” is used in the broadest sense, and includes any molecule that blocks at least in part, a biological activity of an agonist. In some embodiments, the antagonist binds to the agonist, while in other embodiments, the antagonist binds to the ligand of the agonist. For example, an antagonist of the inhibitory immune checkpoint molecule PD-1 binds to and blocks a PD-1 signaling pathway.

The terms “immunostimulatory sequence” and “ISS” refer to a nucleic acid sequence that stimulates a measurable immune response (e.g., measured in vitro, in vivo, and/or ex vivo).

For the purpose of the present disclosure, the term ISS refers to a nucleic acid sequence comprising an unmethylated CG dinucleotide. Examples of measurable immune responses include, but are not limited to, antigen-specific antibody production, cytokine secretion, lymphocyte activation and lymphocyte proliferation.

The terms “CpG” and “CG” are used interchangeably herein to refer to, unless stated otherwise, a cytosine and guanine separated by a phosphate. These terms refer to a linear sequence as opposed to base-pairing of cytosine and guanine. The polynucleotides of the present disclosure contain at least one unmethylated CpG dinucleotide. That is the cytosine in the CpG dinucleotide is not methylated (i.e., is not 5-methylcytosine).

“CpG PNs” or “CpG polynucleotides” of the present disclosure are polynucleotides from 7 to 50 nucleotides in length, which comprise one or more unmethylated CG dinucleotides. In some preferred embodiments, the polynucleotide is an oligodeoxynucleotide (ODN). In some preferred embodiments, the CpG PN includes a TCG at its 5′ end, which imparts the ability to stimulate B cells. In some embodiments, the CpG PN includes a CG-containing palindrome, which imparts the ability to induce human plasmacytoid dendritic cell (PDC) maturation and secretion of high levels of type I interferons (e.g., IFN-α, IFN-γ, etc.). In some embodiments, the CpG PNs are preferably from 12 to 50 nucleotides in length. In some embodiments, the PN is at least (lower limit) 7, 8, 9, 10, 11, 12, 13, 14, 15, 16, 17, 18, 19, 20, 22, 24, 26, 28, 30, 32, 34, 36, 38, 40 or 45 nucleotides in length. In some embodiments, the PN is at most (upper limit) 50, 45, 40, 38, 36, 34, 32, 30, 28, 26, 24, 22 or 20 nucleotides in length. In some embodiments, the at least one palindromic sequence is at least (lower limit) 8, 10, 12, 14, 16, 18, 20, 22, 24, 26, 28, or 30 bases in length. In some embodiments, the at least one palindromic sequence is at most (upper limit) 32, 30, 28, 26, 24, 22, 20, 18, 16, 14, 12 or 10 bases in length. That is, the at least one palindromic sequence can be from 8 to 32 bases in length.

The terms “antisense” and “antisense sequence” as used herein refer to a non-coding strand of a polynucleotide having a sequence complementary to the coding strand of mRNA. In preferred embodiments, the polynucleotides of the present disclosure are not antisense sequences, or RNAi molecules (miRNA and siRNA). That is in preferred embodiments, the polynucleotides of the present disclosure do not have significant homology (or complementarity) to transcripts (or genes) of the mammalian subjects in which they will be used. For instance, a polynucleotide of the present disclosure for modulating an immune response in a human subject is preferably less than 80% identical over its length to nucleic acid sequences of the human genome (e.g., a polynucleotide that is 50 nucleotides in length would share no more than 40 of the 50 bases with a human transcript). That is, in preferred embodiments, the polynucleotides are less than 80%, 75%, 70%, 65%, 60%, 55%, 50%, 45%, 40%, 35%, 30%, 25% or 20%, identical to nucleic acid sequences of mammalian subjects (e.g., such as humans, nonhuman primates, farm animals, dogs, cats, rabbits, rats, mice, etc.) in which they are to be used.

“Stimulation” of a response or parameter includes eliciting and/or enhancing that response or parameter when compared to otherwise same conditions except for a parameter of interest, or alternatively, as compared to another condition (e.g., increase in TLR-signaling in the presence of a TLR agonist as compared to the absence of the TLR agonist). For example, “stimulation” of an immune response means an increase in the response.

“Inhibition” of a response or parameter includes blocking and/or suppressing that response or parameter when compared to otherwise same conditions except for a parameter of interest, or alternatively, as compared to another condition (e.g., decrease in PD-1-signaling in the presence of a PD-1 ligand and a PD-1 antagonist as compared to the presence of the PD-1 ligand in the absence of the PD-1 antagonist). For example, “inhibition” of an immune response means a decrease in the response.

An “effective amount” of an agent disclosed herein is an amount sufficient to carry out a specifically stated purpose. An “effective amount” may be determined empirically in relation to the stated purpose. An “effective amount” or an “amount sufficient” of an agent is that amount adequate to affect a desired biological effect, such as a beneficial result, including a beneficial clinical result. The term “therapeutically effective amount” refers to an amount of an agent (e.g., polynucleotide TLR9 agonist) effective to “treat” a disease or disorder in a subject (e.g., a mammal such as a human). An “effective amount” or an “amount sufficient” of an agent may be administered in one or more doses.

The terms “treating” or “treatment” of a disease refer to executing a protocol, which may include administering one or more drugs to an individual (human or otherwise), in an effort to alleviate a sign or symptom of the disease. Thus, “treating” or “treatment” does not require complete alleviation of signs or symptoms, does not require a cure, and specifically includes protocols that have only a palliative effect on the individual. As used herein, and as well-understood in the art, “treatment” is an approach for obtaining beneficial or desired results, including clinical results. Beneficial or desired clinical results include, but are not limited to, alleviation or amelioration of one or more symptoms, diminishment of extent of disease, stabilized (i.e., not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival of an individual not receiving treatment. “Palliating” a disease or disorder means that the extent and/or undesirable clinical manifestations of the disease or disorder are lessened and/or time course of progression of the disease or disorder is slowed, as compared to the expected untreated outcome. Further, palliation and treatment do not necessarily occur by administration of one dose, but often occur upon administration of a series of doses.

The term “aluminum salts” as used herein, refer to a class of aluminum-containing inorganic chemical compounds suitable for use as a vaccine adjuvant to increase the desired immune response to a vaccine antigen (e.g., generating antibodies or inducing cell-mediated immunity against a simultaneously administered antigen; see, e.g., Lindblad, 2004 Vaccine 22:3658-3668). When used in vaccines, aluminum salts are typically wet-gel suspensions of irregularly-shaped and sized particles that possess crystalline structures of any one of several polymorphs. Antigens adsorb to the particles by several mechanisms, including electrostatic interactions, ligand exchange, and/or hydrophobic interactions. Aluminum salts commonly used as vaccine adjuvants include for instance, aluminum hydroxide (e.g., ALHYDROGEL® 1.3%, ALHYDROGEL® 2% and ALHYDROGEL® 85 adjuvants marketed by Brenntag Biosector A/S), aluminum oxide hydroxide, and aluminum phosphate (e.g., ADJU-PHOS® marketed by Brenntag Biosector A/S). Although, ALHYDROGEL® 85 was employed in exemplary methods, the present disclosure is in no way limited to the use of this brand of aluminum hydroxide adjuvant. Other brands and non-branded aluminum-containing adjuvants are suitable for use in the methods and compositions described herein, provided they have comparable physiochemical properties. Such properties, which are compatible with incorporation into human vaccines, include: 500-10,000 nm diameter size range; porous crystalline structure; and net surface charge (see, e.g., Hem and HogenEsch, 2007 Expert Rev Vaccines, 6:685-698). Aluminum salts suitable for use in the compositions and methods of the present disclosure are aluminum hydroxide salts (also referred to herein as “alum”), which have a net positive charge capable of adsorbing polynucleotides and polypeptides having an overall negative charge.

### II. Compositions and Synthesis of Particles Comprising a Toll Like Receptor 9 (TLR9) Agonist and a Tumor Antigen

Particles of the present disclosure comprise a TLR9 agonist, in which the TLR9 agonist comprises a polynucleotide comprising the sequence 5′-TCGNs-3′ (SEQ ID NO:1), wherein each N is an independently selected nucleoside and s=4 to 47. Exemplary TLR9 agonists are provided in Table S1-1 and may be present as a polynucleotide or chimeric compound thereof. In some embodiments, the TLR9 agonist is a polynucleotide consisting of a polynucleotide sequence selected from group consisting of SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, and SEQ ID NO:73.

In some embodiments, the TLR9 agonist is a polynucleotide consisting of 5′-TCGNqAACGTTCGAACGTTCGAANr-3′ (SEQ ID NO:4), wherein each N is an independently selected nucleoside, q=0, 1, 2, 3, 4 or 5, and r=0 to 29. In some embodiments, the TLR9 agonist is a polynucleotide consisting of 5′-TCG AAC GTT CGA ACG TTC GAA CGT TCG AAT-3′ (SEQ ID NO:6).

In other embodiments, the TLR9 agonist is a chimeric compound of the formula Nu1-Sp1-Nu2-Sp2-Nu3, wherein Nu1, Nu2 and Nu3 are independently selected nucleic acid moieties from 7 to 50 nucleotides in length, and Nu1 consists of the sequence 5′-TCGNs-3′ where s is 4 to 47, wherein Sp1 and Sp2 are the same or different non nucleic acid spacer moieties comprising at least one member of the group consisting of hexaethylene glycol (HEG), triethylene glycol (TEG), propyl, butyl and hexyl, and wherein Sp1 is covalently linked to Nu1 and Nu2, and Sp2 is covalently linked to Nu2 and Nu3. In some embodiments, Nu2 and/or Nu3 of the chimeric compound consist of the sequence 5′-AACGTTNm-3′ where m=1 to 44 (SEQ ID NO:73). In some embodiments, Nu2 of the chimeric compound consists of the sequence 5′-AACGTTNm-3′ where m=1 to 44 (SEQ ID NO:73). In some embodiments, Nu3 of the chimeric compound consists of the sequence 5′-AACGTTNm-3′ where m=1 to 44 (SEQ ID NO:73). In some embodiments, Nu2 and Nu3 of the chimeric compound consist of the sequence 5′-AACGTTNm-3′ where m=1 to 44 (SEQ ID NO:73). In some embodiments, Sp1 and Sp2 are hexaethylene glycol (HEG).

In some preferred embodiments, he TLR9 agonist is:

(SEQ ID NO: 5)

5′-TCGGCGC-3′-HEG-5′-AACGTTC-3′-HEG-5′-TCGGCGC-3′,
or

(SEQ ID NO: 72)

5′ TCGCCGG-3′-HEG-5′-AACGTTC-3′-HEG-5′-TCGCCGG-
3′.

Particles of the present disclosure also comprise a tumor antigen, in which the tumor antigen is a polypeptide from 8 to 1800 amino acids, 9 to 1000 amino acids, or 10 to 100 amino acids. Specifically, the tumor antigen comprises the amino acid sequence of at least one full length protein or fragment thereof. Suitable tumor antigens have been described in the art (see, e.g., Cheever et al., 2009 Clinical Cancer Research, 15:5323-5337; and Caballero and Chen, 2009, Cancer Science, 100:2014-2021). For instance, suitable tumor antigens include but are not limited to WT1, MUC1, LMP2, HPV E6, HPV E7, EGFRvIII, Her-2/neu, idiotype, MAGE A3, p53, NY-ESO-1 (CTAG1B), PSMA, GD2, CEA, MelanA/Mart1, Ras, gp100, proteinase 3, bcr-able, tyrosinase, survivin, PSA, hTERT, sarcoma translocation breakpoints, EphA2, PAP, MP-IAP, AFP, EpCAM, ERG, NA17-A, PAX3, ALK, androgen receptor, cyclin B1, MYCN, PhoC, TRP-2, mesothelin, PSCA, MAGE A1, CYP1B1, PLAC1, BORIS, ETV6-AML, NY-BR-1, RGS5, SART3, carbonic anhydrase IX, PAX5, OY-TES1, sperm protein 17, LCK, HMWMAA, AKAP-4, SSX2, XAGE 1, B7-H3, legumain, Tie 2, Page4, VEGFR2, MAD-CT-1, FAP, PAP, PDGFR-beta, MAD-CT-2, CEA, TRP-1 (gp75), BAGE1, BAGE2, BAGE3, BAGE4, BAGE5, CAMEL, MAGE-A2, MAGE-A4, MAGE-A5, MAGE-A6, MAGE-A8, MAGE-A9, MAGE-A10, MAGE-A11, MAGE-A12, and Fos-related antigen 1. The amino acid sequences of representative tumor antigens are catalogued in the UniProtKB database under the accessions numbers listed in Table I, and incorporated by reference herein.

TABLE I
Tumor Antigens

| | | | UniProtKB |
|---|---|---|---|
| Tumor Antigen | Protein | Gene | Accession No. |
| WT-1 | Wilms tumor protein | WT1 | P19544 |
| MUC-1 | Mucin-1 | MUC1 | P15941 |
| LMP2 | Latent membrane protein 2 | LMP2 | P13285 |
| HPV E6 | HPV Protein E6 | E6 | P03126 |
| HPV E7 | HPV Protein E7 | E7 | P03129 |
| EGFRvIII | Epidermal growth factor receptor | EGRF | P00533 |
| Her-2/neu | Receptor tyrosine-protein kinase erbB-2 | ERBB2 | P04626 |
| MAGE A1 | Melanoma-associated antigen 1 | MAGEA1 | P43355 |
| MAGE A2 | Melanoma-associated antigen 2 | MAGEA2 | P43356 |
| MAGE A3 | Melanoma-associated antigen 3 | MAGEA3 | P43357 |
| MAGE A4 | Melanoma-associated antigen 4 | MAGEA4 | Q1RN33 |
| MAGE A5 | Melanoma-associated antigen 5 | MAGEA5 | P43359 |
| MAGE A6 | Melanoma-associated antigen 6 | MAGEA6 | P43360 |
| MAGE A8 | Melanoma-associated antigen 8 | MAGEA8 | P43361 |
| MAGE A9 | Melanoma-associated antigen 9 | MAGEA9 | P43362 |
| MAGE A10 | Melanoma-associated antigen 10 | MAGEA10 | P43363 |
| MAGE A11 | Melanoma-associated antigen 11 | MAGEA11 | P43364 |
| MAGE A12 | Melanoma-associated antigen 12 | MAGEA12 | Q6FHH8 |
| p53 | Cellular tumor antigen p53 | TP53 | P04637 |
| NY-ESO-1 | Cancer/testis antigen 1 | CTAG1A | P78358 |
| PSMA | Glutamate carboxypeptidase 2 | FOLH1 | Q04609 |
| CEA | Carcinoembryonic antigen-related cell | CEACAM1 | P13688 |
| | adhesion molecule 1 | | |
| MelanA/Mart1 | Melanoma antigen recognized by T-cells 1 | MLANA | Q16655 |
| Ras | GTPase KRas | KRAS | P01116 |
| gp100 | Melanocyte protein PMEL | PMEL | P40967 |
| Proteinase 3 | Proteinase 3 | PRTN3 | D6CHE9 |
| bcr-able | Tyrosine-protein kinase ABL1 | ABL1 | P00519 |
| tyrosinase | Tyrosinase | TYR | P14679 |
| survivin | Baculoviral IAP repeat-containing | BIRC5 | O15392 |
| | protein 5 | | |
| PSA | Prostate-specific antigen | KLK3 | P07288 |
| hTERT | Telomerase reverse transcriptase | TERT | O14746 |
| sarcoma | RNA-binding protein EWS | EWSR1 | Q01844 |
| translocation | | | |
| breakpoints | | | |
| EphA2 | Ephrin type-A receptor 2 | EPHA2 | P29317 |
| PAP | Prostatic acid phosphatase | ACPP | P15309 |
| MP-IAP | Baculoviral TAP repeat-containing | BIRC7 | Q96CA5 |
| | protein 7 | | |
| AFP | Alpha-fetoprotein | AFP | P02771 |
| EpCAM | Epithelial cell adhesion molecule | EPCAM | P16422 |
| ERG | Transcriptional regulator ERG | ERG | P11308 |
| NA17-A | Alpha-1,6-mannosylglycoprotein 6-beta- | MGAT5 | Q09328 |
| | N-acetylglucosaminyltransferase A | | |
| PAX3 | Paired box protein Pax-3 | PAX3 | P23760 |
| ALK | ALK tyrosine kinase receptor | ALK | Q9UM73 |
| androgen | Androgen receptor | AR | P10275 |
| receptor | | | |
| cyclin B1 | G2/mitotic-specific cyclin-B1 | CCNB1 | P14635 |
| MYCN | N-myc proto-oncogene protein | MYCN | P04198 |
| PhoC | | | |
| TRP-2 | L-dopachrome tautomerase | DCT | P40126 |
| mesothelin | Mesothelin | MSLN | Q13421 |
| PSCA | Prostate stem cell antigen | PSCA | O43653 |
| CYP1B1 | Cytochrome P450 1B1 | CYP1B1 | Q16678 |
| PLAC1 | Placenta-specific protein | PLAC1 | Q9HBJ0 |
| BORIS | Transcriptional repressor CTCFL | CTCFL | Q8NI51 |
| ETV6-AML | Transcription factor ETV6 | ETV6 | P41212 |
| NY-BR-1 | Ankyrin repeat domain-containing | ANKRD30A | Q9BXX3 |
| | protein 30A | | |
| RGS5 | Regulator of G-protein signaling 5 | RGS5 | O15539 |
| SART3 | Squamous cell carcinoma antigen | SART3 | Q15020 |
| | recognized by T-cells 3 | | |
| carbonic | Carbonic anhydrase 9 | CA9 | Q16790 |
| anhydrase IX | | | |
| PAX5 | Paired box protein Pax-5 | PAX5 | Q02548 |
| OY-TES1 | Acrosin-binding protein | ACRBP | Q8NEB7 |
| sperm protein 17 | Sperm surface protein Sp17 | SPA17 | Q15506 |
| LCK | Tyrosine-protein kinase Lck | LCK | P06239 |
| HMWMAA | Chondroitin sulfate proteoglycan 4 | CSPG4 | Q6UVK1 |
| AKAP-4 | A-kinase anchor protein 4 | AKAP4 | Q5JQC9 |
| SSX2 | Protein SSX2 | SSX2 | Q16385 |
| XAGE 1 | X antigen family member 1 | XAGE 1 | Q9HD64 |
| B7-H3 | CD276 antigen | CD276 | Q5ZPR3 |
| legumain | Legumain | LGMN | Q99538 |
| Tie 2 | Angiopoietin-1 receptor | TEK | Q02763 |
| Page4 | P antigen family member 4 | PAGE4 | O60829 |
| VEGFR2 | Vascular endothelial growth factor | KDR | P35968 |
| | receptor 2 | | |
| MAD-CT-1 | Sperm protamine P1 | PRM1 | P04553 |
| MAD-CT-2 | Protamine-2 | PRM2 | P04554 |
| FAP | Prolyl endopeptidase FAP | FAP | Q12884 |
| PAP | Prostatic acid phosphatase | ACPP | P15309 |
| PDGFR-beta | Platelet-derived growth factor receptor | PDGFRB | P09619 |
| | beta | | |
| CEA | Carcinoembryonic antigen-related cell | CEACAM5 | P06731 |
| | adhesion molecule 5 | | |
| TRP-1 (gp75) | 5,6-dihydroxyindole-2-carboxylic acid | TYRP1 | P17643 |
| | oxidase | | |
| BAGE1 | B melanoma antigen 1 | BAGE1 | Q13072 |
| BAGE2 | B melanoma antigen 2 | BAGE2 | Q86Y30 |
| BAGE3 | B melanoma antigen 3 | BAGE3 | Q86Y29 |
| BAGE4 | B melanoma antigen 4 | BAGE4 | Q86Y28 |
| BAGE5 | B melanoma antigen 5 | BAGE5 | Q86Y27 |
| CAMEL | CTL-recognized antigen on melanoma | CAMEL | O95987 |
| Fos-related | Fos-related antigen 1 | FOSL1 | P15407 |
| antigen 1 | | | |

In some preferred embodiments, the tumor antigen comprises an amino acid sequence or fragment thereof from one or more of the group consisting of gp100, hTERT, MAGE A1, MAGE A3, MAGE A10, MelanA/Mart1, NY-ESO-1 (CTAG1B), PSA, Ras, survivin, TRP1 (gp75), TRP2, and tyrosinase. In some embodiments, the tumor antigen comprises a mammalian antigen (e.g., Triple peptide) or a viral antigen (e.g., HPV1 E6 and/or HPV E7) expressed by the tumor. In some embodiments, the mammalian antigen is a neoantigen or encoded by a gene comprising a mutation relative to the gene present in normal cells from the mammalian subject. Neoantigens are thought to be particularly useful in enabling T cells to distinguish between cancer cells and non-cancer cells (see, e.g., Schumacher and Schreiber, 2015 Science 348:69-74; Desrichard et al., 2016 Clinical Cancer Res, 22:807-812; Wang and Wang, 2017 Cell Research 27:11-37).

In some embodiments, the tumor antigen is a fusion protein comprising two or more polypeptides, wherein each polypeptide comprises an amino acid sequence from a different tumor antigen or non-contiguous amino acid sequences from the same tumor antigen. In some of these embodiments, the fusion protein comprises a first polypeptide and a second polypeptide, wherein each polypeptide comprises non-contiguous amino acid sequences from the same tumor antigen.

In some embodiments, the polypeptide is modified to include a single cysteine residue at either the N- or C-terminus to enable covalent linkage via the thiol group of the cysteine. In other instances from one to three amino acid residues, or non-natural amino acid residues, are added to one or both of the N-terminus and the C-terminus of the polypeptide antigen to create a modified polypeptide antigen to enable a covalent linkage via a number of bioconjugate chemistries known in the art. The present disclosure provides an immunogenic composition comprising any particles detailed herein, for example a particle comprising a TLR9 agonist and a tumor antigen each associated with a biocompatible multimerization agent, wherein:
the multimerization agent has a diameter of 10 to 10,000 nanometers and/or a molecular weight of about 10,000 to about 1,000,000 Daltons;
the TLR9 agonist comprises a polynucleotide comprising the sequence 5′-TCGNs-3′ (SEQ ID NO:1), wherein each N is an independently selected nucleoside, s=4 to 47;
the tumor antigen comprises a polypeptide of about 9 to about 1000 amino acids; and
the TLR9 agonist and the tumor antigen are either each associated with the multimerization agent by one or more covalent linkages, or each associated with the multimerization agent by adsorption.

### A. TLR9 Agonist:Aluminum Salt Complex:Tumor Antigen Co-Adsorbates

The present disclosure provides methods for preparing particles containing a TLR9 agonist and a tumor antigen, as well as compositions and intermediates useful therein.

In one aspect, the disclosure provides a method for preparing a particle comprising a TLR9 agonist and a tumor antigen each associated with a biocompatible multimerization agent (e.g., an aluminum salt complex) by adsorption, the method comprising mixing the tumor antigen and the TLR9 agonist with the aluminum salt complex equilibrated in a buffer having a pH of about 6 to 9, preferably about 7 to 8. In order to enhance dissolution, the tumor antigen is dissolved in 5% to 30% (preferably 10% to 20%) of an organic solvent in water or a buffer. Suitable organic solvents include but are not limited to acetic acid, acetone, anisole, 1-butanol, 2-butanol, butyl acetate, tert-butyl methyl ether, cumene, dimethyl sulfoxide, ethanol, ethyl acetate, ethyl ether, ethyl formate, formic acid, heptane, isobutyl acetate, isopropyl acetate, methyl acetate, 3-methyl-1-butanol, methylethylketone, methylisobutylketone, 2-methyl-1-propanol, pentane, 1-pentanol, 1-propanol, 2-propanol (isopropanol), propyl acetate, and combinations thereof. In some preferred embodiments, the tumor antigen is dissolved in 5% to 30% (preferably 10% to 20%) isopropanol in water or a buffer. The TLR9 agonist may also be dissolved in a buffer. For efficient adsorption, phosphate buffer should be avoided. The tumor antigen and the TLR9 agonist may be adsorbed to the multimerization agent (e.g., the aluminum salt complex) simultaneously or sequentially. For example, a solution of the tumor antigen may be added to the multimerization agent in a buffer to allow adsorption for a period of time; the TLR9 agonist is then added to the mixture for adsorption; or a solution of the TLR9 agonist may be added to the multimerization agent in a buffer to allow adsorption for a period of time; the tumor antigen is then added to the mixture for adsorption. Alternatively, a solution of the tumor antigen and the TLR9 agonist may be added at the same time, or one is added shortly after the other, to allow adsorption of the tumor antigen and the TLR9 agonist on to the multimerization agent (e.g., the aluminum salt complex) at the same time.

In some embodiments, provided is a method for preparing a particle comprising a TLR9 agonist and a tumor antigen each associated with a biocompatible multimerization agent by adsorption, wherein
the multimerization agent is an aluminum salt complex (e.g., an aluminum hydroxide complex or ALHYDROGEL®), having particle diameter of 100 to 25,000 nanometers, 500 to 25,000 nanometers or 1 to 25 micrometers,
the TLR9 agonist comprises a polynucleotide comprising the sequence 5′-TCGNs-3′ (SEQ ID NO:1), wherein s=4 to 47 and each N is a nucleoside, and
the tumor antigen comprises a polypeptide of about 8 to 1800 amino acids, 9 to about 1000 amino acids, or about 10 to about 100 amino acids,
   the method comprising:
adding the tumor antigen dissolved in an aqueous solution containing about 5% to about 30% (preferably about 10% to about 20%) isopropanol or other organic solvent, and the TLR9 agonist, to the aluminum salt complex equilibrated in a buffer,
wherein the buffer is in a pH range of about 6 to about 9 (preferably about 7 to about 8) and the buffer is not a phosphate buffer.

In some embodiments, the aluminum salt complex comprises an aluminum hydroxide complex (e.g., ALHYDROGEL®). In some embodiments, the tumor antigen and the TLR9 agonist are adsorbed to the aluminum salt complex at the same time. In some embodiments, the tumor antigen is adsorbed to the aluminum salt complex first followed by adsorption of the TLR9 agonist. In some embodiments, the TLR9 agonist is adsorbed to the aluminum salt complex first followed by adsorption of the tumor antigen.

Any suitable buffer in the pH range of about 6 to 9 may be used for aluminum salt complex binding reactions in the method, for example, any non-phosphate buffer. Phosphate buffers are generally avoided because they may compete for binding sites on the aluminum salt complex, diminishing the loading capacity. Additionally, exposure of the ligand:aluminum salt complex to phosphate may cause phosphate ligand exchange where the ligand is displaced by phosphate. See Lindblad, Immunology and Cell Biology 2004, 82, 497-505. In some embodiments, the buffer is an acetate buffer (e.g., a pH ˜7 sodium acetate buffer). In some embodiments, the buffer is a bicarbonate buffer (e.g., a pH ˜8 sodium bicarbonate buffer). In some embodiments, the TLR9 agonist is dissolved in a non-phosphate buffer. In some embodiments, the aluminum salt complex (e.g., an aluminum hydroxide complex or ALHYDROGEL®) is equilibrated in a non-phosphate buffer. Suitable non-phosphate buffers include but are not limited to acetate buffers, bicarbonate buffers, borate buffers, carbonate buffers, citrate buffers, glycine buffers, phthalate buffers, tetraborate buffers, and TRIS buffers.

Numerous protein and peptide antigens have been efficiently and stably adsorbed to aluminum adjuvants and used in research and for clinical application as adjuvants for vaccines in both animals and humans over many decades. See Aebig et al., J. Immunol. Methods 2007, 323(2):139-146. In general, the association between antigen and aluminum adjuvants is thought to be driven mostly by electrostatic forces; however hydrogen bonds, van der Waals interactions, and hydrophobic, hydrophobic interactions may also play a role. Therefore, binding capacity and efficiency is expected to depend upon the unique properties of each peptide; e.g., sequence, solubility, structure, charge and hydrophobicity as well as binding conditions, including buffer type and composition, peptide concentration, ionic strength, pH, time and temperature.

Unlike CpG-ODNs, many peptide antigens of interest are highly hydrophobic and are not soluble at useful concentrations in aqueous buffers commonly used for binding to aluminum salt complex. The present disclosure provides a co-solvent system using one or more organic solvents added to the aqueous buffer (e.g., acetic acid, acetone, anisole, 1-butanol, 2-butanol, butyl acetate, dimethyl sulfoxide [DMSO], ethanol, formic acid, isopropanol, 2-propanol, acetonitrile, 1,2-dichloroethane, N,N-dimethylformamide, trifluoroacetic acid, and combinations thereof) that results in higher binding efficiencies and binding capacities of peptide than when an all aqueous system is used. In exemplary embodiments, isopropanol is used as a co-solvent in order to enhance dissolution of the tumor antigen, thus facilitating adsorption of the tumor antigen to the multimerization agent. The amount of isopropanol required may depend on the nature of the tumor antigen and the multimerization agent. For example, a more hydrophobic tumor antigen polypeptide tends to require more isopropanol for dissolution and efficient adsorption than a less hydrophobic tumor antigen polypeptide. Use of the isopropanol co-solvent system can apply to many different types of peptides with different hydrophobicities. Preferred embodiments of an organic solvent include, but are not limited to, isopropanol, DMSO, ethanol, formic acid, and acetic acid.

Any amount of isopropanol (or other suitable organic solvent) may be used between the amount required to dissolve the polypeptide (minimum) and the amount that may cause dehydration or dissolution of the aluminum salt (maximum). Appropriate alternatives to isopropanol include but are not limited to: acetic acid, acetone, anisole, 1-butanol, 2-butanol, butyl acetate, tert-butyl methyl ether, cumene, dimethyl sulfoxide, ethanol, ethyl acetate, ethyl ether, ethyl formate, formic acid, heptane, isobutyl acetate, isopropyl acetate, methyl acetate, 3-methyl-1-butanol, methylethylketone, methylisobutylketone, 2-methyl-1-propanol, pentane, 1-pentanol, 1-propanol, and propyl acetate. In some embodiments, the tumor antigen is dissolved in an aqueous solution containing about 5% to about 30%, about 5% to about 25%, about 5% to about 20%, about 5% to about 15%, about 5% to about 10%, about 10% to about 30%, about 10% to about 25%, about 10% to about 20%, about 10% to about 15%, about 15% to about 20%, about 15% to about 25%, about 15% to about 30%, about 20% to about 30%, or about 12% to about 18% isopropanol. In some embodiments, the tumor antigen is dissolved in an aqueous solution containing about 5%, about 8%, about 10%, about 11%, about 13%, about 14%, about 15%, about 16%, about 17%, about 18%, about 19%, about 20%, about 25%, or about 30% isopropanol. In some embodiments, the tumor antigen is dissolved in about 10% to about 20% isopropanol, in water or an aqueous buffer. In some embodiments, the tumor antigen is dissolved in about 5% to about 30%, preferably about 10% to about 20%, isopropanol, in water. In some embodiments, the tumor antigen is dissolved in about 5% to about 30%, preferably about 10% to about 20%, isopropanol, in an aqueous buffer (e.g., a pH˜8 sodium bicarbonate buffer).

In some embodiments, the method for preparing a microparticle comprising a TLR9 agonist and a tumor antigen each associated with a biocompatible multimerization agent by adsorption further comprises one of more of the following steps: (i) dissolving the tumor antigen in an aqueous solution containing about 5% to about 30% (preferably about 10% to about 20%) isopropanol, (ii) dissolving the TLR9 agonist in water or a non-phosphate buffer, and (iii) pre-equilibrating the aluminum salt complex (e.g., an aluminum hydroxide complex or ALHYDROGEL®) in a non-phosphate buffer.

The amount of the TLR9 agonist (e.g., a CpG-ODN) and the tumor antigen (e.g., a peptide antigen) used relative to the amount of multimerization agent (e.g., an aluminum hydroxide complex or ALHYDROGEL®) can be adjusted to provide desirable ratios of TLR9 agonist:aluminum hydroxide:tumor antigen in the co-adsorbates. In some embodiments, the ratio of the TLR9 agonist (e.g., a CpG-ODN) to the multimerization agent (e.g., an aluminum hydroxide complex or ALHYDROGEL®) by weight is between about 0.2:1 and about 2:1, between about 0.4:1 and about 2:1, between about 0.6:1 and about 2:1, between about 0.8:1 and about 2:1, between about 0.2:1 and about 3:1, between about 0.2:1 and about 4:1, or between about 0.2:1 and about 5:1. In some preferred embodiments, the TLR9 agonist:aluminum hydroxide (w/w) ratio is in the range of about 0.2 to about 1.2. In some embodiments, the TLR9 agonist:aluminum hydroxide (w/w) ratio is greater than (lower limit) about 0.2:1, about 0.3:1, about 0.4:1, about 0.5:1, about 0.6:1, about 0.7:1, about 0.8:1, about 0.9:1, about 1:1, about 1.2:1, about 1.5:1, about 2.0:1, about 2.5:1, about 3.0:1, about 4:1, or about 5:1. In some embodiments, the TLR9 agonist:aluminum hydroxide (w/w) ratio is less than (upper limit) about 5:1, about 4:1, about 3:1, about 2.5:1, about 2:1, about 1.8:1, about 1.5:1, about 1.2:1, about 1:1, about 0.9:1, about 0.8:1, about 0.7:0 or about 0.6:1. That is, the TLR9 agonist:aluminum hydroxide (w/w) ratio is in the range of about 0.2:1 to about 5:1 in which the lower limit is less than the upper limit. In some embodiments, the TLR9 agonist:aluminum hydroxide (w/w) ratio is about 0.4:1, about 0.6:1, about 0.8:1, about 1:1, or about 1.2:1.

In some embodiments, the ratio of the tumor antigen (e.g., peptide antigen(s)) to the multimerization agent (e.g., an aluminum hydroxide complex or ALHYDROGEL®) by weight is between about 0.01:1 and about 2:1, between about 0.05:1 and about 2:1, between about 0.1:1 and about 2:1, between about 0.2:1 and about 2:1, between about 0.4:1 and about 2:1, between about 0.6:1 and about 2:1, between about 0.8:1 and about 2:1, between about 1.0:1 and about 2:1, or between about 1.5:1 and about 2:1. In some preferred embodiments, the tumor antigen:aluminum hydroxide (w/w) ratio is in the range of about 0.005:1 to about 2:1. In some embodiments, the tumor antigen:aluminum hydroxide (w/w) ratio is greater than (lower limit) about 0.005:1, about 0.01:1, about 0.05:1, about 0.1:1, about 0.2:1, about 0.4:1, about 0.5:1, about 0.6:1, about 0.8:1, about 1:1, about 1.2:1 or about 1.5:1. In some embodiments, each of the tumor antigens:aluminum hydroxide (w/w) ratio is less than (upper limit) about 2:1, about 1.8:1, about 1.5:1, about 1.2:1, about 1:1, about 0.8:1, or about 0.6:1. That is, the tumor antigen:aluminum hydroxide (w/w) ratio is in the range of about 0.2:1 to about 2:1, or about 0.005:1 to about 2:1 in which the lower limit is less than the upper limit. In some embodiments, the tumor antigen:aluminum hydroxide (w/w) ratio is about 0.4:1, about 0.6:1, about 0.8:1, about 1:1, or about 1.2:1.

In some embodiments, appropriate amounts of the TLR9 agonist (e.g., a CpG-ODN), the tumor antigen (e.g., peptide antigen(s)) and the multimerization agent (e.g., an aluminum hydroxide complex or ALHYDROGEL®) are mixed to provide a co-adsorbate having a TLR9 agonist:aluminum hydroxide:tumor antigen (w/w/w) ratio of between about 0.4:1:0.4, to about 2:1:2, preferably between about 0.6:1:0.6 and about 1.2:1:1.2. In some embodiments, the co-adsorbate TLR9 agonist:aluminum hydroxide:tumor antigen (w/w/w) ratio is between about 0.2:1:0.01 to about 5:1:2, preferably between about 1:1:0.05 and about 4:1:0.6. The TLR9 agonist:aluminum hydroxide (w/w) ratio and the tumor antigen:aluminum hydroxide (w/w) ratio in the adsorbate may be different or the same. For example, in some embodiments, the TLR9 agonist:aluminum hydroxide:tumor antigen (w/w/w) ratio in the adsorbate is about 0.8:1:1.3. In some embodiments, the TLR9 agonist:aluminum hydroxide:tumor antigen (w/w/w) ratio in the adsorbate is about 1:1:1.

### B. TLR9 Agonist Polysaccharide-Tumor Antigen Co-Conjugates

In one aspect, the disclosure provides a method for preparing particles comprising a TLR9 agonist and a tumor antigen each covalently linked to a biocompatible multimerization agent (e.g., a polysaccharide), the method comprising reacting a TLR9 agonist comprising or functionalized with a thiol group, and reacting a tumor antigen comprising a thiol group (e.g., a cysteine thiol or a alkyl-thiol group from functionalizing the tumor antigen peptide), with a polysaccharide functionalized with a maleimide group. The tumor antigen and the TLR9 agonist may be covalently linked to the polysaccharide simultaneously or sequentially. For example, a TLR9 agonist comprising or functionalized with a thiol group may be allowed to react with some of the maleimide groups linked to the polysaccharide, and a tumor antigen is then reacted with the remaining maleimide groups linked to the polysaccharide. The tumor antigen may also be allowed to react with the maleimide groups in the polysaccharide first, and the TLR9 agonist is then allowed to react with the remaining maleimide groups in the polysaccharide. Alternatively, the polysaccharide functionalized with a maleimide group and the tumor antigen comprising a thiol group may be allowed to react with polysaccharide functionalized with maleimide groups at the same time. In some instances, the reactions are carried out in a buffer to control the pH of the reaction mixture. Inclusion of guanidine hydrochloride in the buffer aids dissolution of the tumor antigen, especially hydrophobic tumor antigens.

In some embodiments, provided is a method for preparing a TLR9 agonist-polysaccharide-tumor antigen co-conjugate compound of formula (I):

[D-L¹-L²-(PEG)-L³]ₓ-F-[L³-(PEG)-L²-A]ₜ (I),

wherein:
D is a TLR9 agonist;
L¹ is a first linker comprising an alkylthio group;
L² is a second linker comprising a succinimide group;
L³ is a third linker comprising an amide group;
PEG is a polyethylene glycol (e.g., —(OCH₂CH₂)ₙ—, where n is an integer from 2 to 80);
t and x are independently an integer from 3 to 200;
A is a tumor antigen; and
F is a polysaccharide having a molecular weight of about 10,000 to about 1,000,000 Daltons and is connected to L³ via an ether group,
wherein the TLR9 agonist comprises a polynucleotide comprising the sequence 5′-TCGNs-3′, wherein s=4 to 47 and each N is a nucleoside, and wherein one or more linkages between the nucleotides and between the 3′-terminal nucleotide and L¹ are phosphorothioate ester linkages, and
wherein A is a tumor antigen comprising a polypeptide of about 9 to about 1000 amino acids and comprises at least one thiol group,
wherein the method comprises:
reacting a compound of the formula D-L^{1a}-SH, where D is as defined for formula (I) and L^{1a} is (CH₂)ₘ where m is an integer from 2 to 9, and reacting a compound of the formula A, with a compound of formula (II):

[L^{2a}-(PEG)-L³]_{y}-F (II)
wherein L³, PEG and F are as defined for formula (I);
L^{2a} is

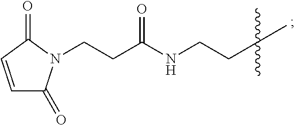 [SMILES: *CCNC(=O)CCN1C(=O)C=CC1=O]

and
y is an integer from 6 to 350; provided that y is no less than the sum of t and x (y>=t+x).

In some embodiments, the method comprises reacting a compound of the formula D-L^{1a}-SH with a compound of formula (II) to form an intermediate, and reacting a compound of the formula A with the intermediate. In some embodiments, the method comprises reacting a compound of the formula A with a compound of formula (II) to form an intermediate, and reacting a compound of the formula D-L^{1a}-SH with the intermediate. In some embodiments, the reaction is carried out in a medium (e.g., a buffer) comprising guanidine hydrochloride.

The number of TLR9 agonist D and tumor antigen A in the TLR9 agonist-polysaccharide-tumor antigen co-conjugate compound of formula (I) can range independently from 3 to about 200. That is, x and t are independently an integer from 3 to 200. In some embodiments, x and t are independently an integer greater than (lower limit) 3, 6, 9, 12, 15, 20, 25, 30, 35, 40, 45, 50, 55, 60, 65, 70, 75, 80, 90, 100, 110, 120, 130, 140, or 150. In some embodiments, x and t are independently an integer less than (upper limit) 200, 190, 180, 160, 150, 140, 130, 120, 110, 100, 90, 80, 75, 70, 65, 60, 55, 50, 45, or 40. That is x and t can independently be an integer in the range of from about 3 to 200 in which the lower limit is less than the upper limit. For example, in some embodiments, x is from 10 to 200, from 10 to 150, from 10 to 120, from 15 to 100, from 15 to 50, from 20 to 120, or from 20 to 40. In some embodiments, x is about 30±10. In a preferred embodiment, x is about 30. In some embodiments, t is from 10 to 200, from 10 to 150, from 10 to 120, from 12 to 100, from 12 to 80, from 20 to 80, or from 35 to 75. In some embodiments, t is about 55±20. In a preferred embodiment, t is about 55.

In some embodiments, the tumor antigen comprises at least one cysteine residue. In some embodiments, the tumor antigen comprises a polypeptide of about 9 to about 1000 amino acids. In some embodiments the least one cysteine residue is located at the N-terminus or the C-terminus of the polypeptide. In some embodiments, the tumor antigen comprises an amino acid sequence of a mammalian antigen expressed by cells of a tumor. In some embodiments, the tumor antigen comprises an amino acid sequence of a viral antigen expressed by the tumor.

In some instances, every maleimide group in the compound of formula (II) is reacted with either a thiol linked to D or a thiol of A. Thus in some embodiments, y=t+x. In other instances, only some of the maleimide groups in the compound of formula (II) are reacted with a thiol linked to D or a thiol of A, while some are not reacted with a thiol linked to D or a thiol of A. Thus in some embodiments, y>t+x. In some embodiments, y is an integer greater than (lower limit) 6, 9, 12, 20, 30, 40, 50, 60, 70, 80, 90, 100, 110, 120, 130, 140, 150, 155, 165, 190 or 200. In some embodiments, y is an integer less than (upper limit) 350, 300, 275, 250, 225, 215, 210, 205, 200, 190, 180, 160, 150, 140, 130, 120, 110, 100, 90, 80, 70, 60 or 50. That is y can be an integer in the range of from about 6 to 350 in which the lower limit is less than the upper limit. For example, in some embodiments, y is from 20 to 350, from 30 to 300, from 155 to 215, from 165 to 205, from 20 to 250, from 90 to 250, from 120 to 250, from 120 to 220, from 160 to 220, from 20 to 200, from 60 to 180, from 90 to 150, from 100 to 140, or from 110 to 130. In a preferred embodiment, y is about 190, about 185, about 150 or about 120. In some embodiments, y is about 190±30 or about 185±30. In some embodiments when y is an integer greater than the sum of t and x, the maleimide groups that are not reacted with a nucleic acid moiety D are capped and/or hydrolyzed. In some embodiments when y is an integer greater than the sum of t and x, the maleimide groups that are not reacted with a nucleic acid moiety D are capped with cysteine and/or are hydrolyzed by water.

The reactive thiol compound D-L^{1a}-SH and the maleimide functionalized polysaccharide of the formula (II) can be prepared using methods described herein and known in the art, for example, methods described in PCT patent application PCT/US2016/014635, filed Jan. 22, 2016 and published as WO 2016/118932, the contents of which is incorporated herein by reference.

The reactive thiol compound D-L^{1a}-SH is often made from a more stable disulfide compound prior to use. In some embodiments, the method further comprises reacting a disulfide compound of the formula D-L^{1a}-SS-L^{1a}-OH with a reducing agent (e.g., a phosphine compound). In some embodiments, D is as defined herein for formula (I) and L^{1a} is (CH₂)ₘ where m is an integer from 2 to 9. In some of the embodiments, m is 2, 3, 4, 5, 6, 7, 8 or 9. In some embodiments, m is from 3 to 6. In some of these embodiments, m is 3 or 6. In one embodiment, m is 6. In one embodiment, m is 3. One example of the reducing agent is tris(2-carboxyethyl)phosphine hydrochloride (TCEP).

Described herein is a compound of the formula D-L^{1a}-SH or a compound of the formula D-L^{1a}-SS-L^{1a}-OH, wherein D is a TLR9 agonist detailed herein, and L^{1a} is (CH₂)ₘ where m is an integer from 2 to 9. In some of these embodiments, D is a polynucleotide comprising a sequence 5′-TCGNs-3′ (SEQ ID NO:1), wherein s=4 to 47 and each N is a nucleoside. In some of the embodiments, m is 2, 3, 4, 5, 6, 7, 8 or 9. In some embodiments, m is from 3 to 6 or m is 3 or 6. In one embodiment, m is 6. In one embodiment, m is 3.

The PEG in the compound of the formula (II) can be introduced via an amine derivative of the multivalent polysaccharide F reacting with an activated ester compound comprising the PEG. In some embodiments, the method of making a compound of formula (I) further comprises reacting a compound of the formula (III):

[NH₂CH₂CH₂NHC(O)CH₂]_{z}-F (III)
wherein F is as defined for formula (I) and z is an integer from 6 to 400,
with a compound of the formula L^{2a}-(PEG)-L^{3a}-Lv, where L^{2a} and PEG are as defined for formula (II); L^{1a} is —NHC(O)CH₂CH₂C(O)— or —C(O)—; and Lv is a leaving group,
to form the compound of the formula (II).

In some embodiments, the activated ester compound comprising the PEG is an N-hydroxysuccinimide (NHS or HOSu) ester, and Lv is (2,5-dioxopyrrolidin-1-yl)oxy (i.e., OSu). Other activated carboxylic acid or esters known in the art can be used to react with the amine of formula (III) to form the compound of formula (II).

In some embodiments, F is a branched copolymer of sucrose and epichlorohydrin having a molecular weight of about 100,000 to 700,000 in Daltons. In some embodiments, F is a branched copolymer of sucrose and epichlorohydrin having a molecular weight of about 400,000±100,000 Daltons (e.g., a FICOLL® PM 400), and the compound of formula (III) is a compound of AECM-FICOLL®400. Depending on the relative amounts of the activated ester L^{2a}-(PEG)-L^{3a}-Lv (e.g., an NHS ester L^{ea}-(PEG)-L^{1a}-OSu) to the compound of formula (III) (e.g., a compound of AECM-FICOLL®400) used, some or all of the amino groups in the compound of formula (III) may be PEGylated. Thus in some embodiments, z equals to y. In some embodiments, z is an integer greater than y. In some embodiments, z is an integer greater than (lower limit) 6, 9, 12, 20, 30, 40, 50, 60, 70, 80, 90, 100, 110, 120, 130, 140, 150, 155, 165, 190 or 200. In some embodiments, z is an integer less than (upper limit) 400, 350, 300, 275, 250, 225, 215, 210, 205, 200, 190, 180, 160, 150, 140, 130, 120, 110, 100, 90, 80, 70, 60 or 50. That z can be an integer in the range of from about 6 to 400 in which the lower limit is less than the upper limit. For example, in some embodiments, z is from 20 to 400, from 50 to 300, from 190 to 250, from 200 to 240, from 20 to 350, from 30 to 300, from 155 to 215, from 165 to 205, from 20 to 250, from 90 to 250, from 120 to 250, from 120 to 220, from 160 to 220, from 20 to 200, from 60 to 180, from 90 to 150, from 100 to 140, or from 110 to 130. In a preferred embodiment, z is about 220, about 190, about 150 or about 120. In some embodiments, z is about 220±30 or about 220±20. In some embodiments when z is an integer greater than y, excess amines are capped. In some embodiments when z is an integer greater than y, excess amines are capped with sulfo-NHS-acetate or NHS-acetate.

FICOLL® is synthesized by cross-linking sucrose with epichlorohydrin which results in a highly branched structure. Aminoethylcarboxymethyl-FICOLL (AECM-FICOLL®) can be prepared by the method of Inman, 1975, J. Imm. 114:704-709. AECM-FICOLL can then be reacted with a heterobifunctional crosslinking reagent, such as 6-maleimido caproic acyl N-hydroxysuccinimide ester, and then conjugated to a thiol-derivatized nucleic acid moiety (see Lee et al., 1980, Mol. Imm. 17:749-56). Other polysaccharides may be modified similarly.

The NHS ester (L^{2a}-(PEG)-L^{1a}-OSu) used in the method may be obtained from commercial sources or made by methods known in the art.

In some embodiments, the method for preparing a compound of formula (I) further comprises:
reacting a compound of the formula L^{2a}-(PEG)-L^{3a}-Lv, where
   L^{2a} is a moiety comprising a maleimide group,
   PEG is a polyethylene glycol (e.g., —(OCH₂CH₂)ₙ—, where n=2 to 80),
   L^{3a} is —NHC(O)CH₂CH₂C(O)—, and
      Lv is a leaving group (e.g., (2,5-dioxopyrrolidin-1-yl)oxy),
with a compound of the formula (III):

[NH₂CH₂CH₂NHC(O)CH₂]_{z}-F (III)
wherein F is a branched copolymer of sucrose and epichlorohydrin and is connected to L³ via an ether group, and
z is independently an integer from 6 to 400;
to produce a compound of formula (II):

[L^{2a}-(PEG)-L³]_{y}-F (II),
where y is an integer from 6 to 350.

The present disclosure also provides a method for preparing a composition comprising a distribution of compounds of formula (I) detailed herein from a distribution of compounds of the formula (II). In one aspect, provided is a method for preparing a composition comprising compounds of formula (I):

[D-L¹-L²-(PEG)-L³]ₓF-[L³-(PEG)-L²-A]ₜ (I),

wherein:
D is a TLR9 agonist;
L¹ is a first linker comprising an alkylthio group;
L² is a second linker comprising a succinimide group;
L³ is a third linker comprising an amide group;
PEG is a polyethylene glycol (e.g., —(OCH₂CH₂)ₙ—, where n is an integer from 2 to 80);
t and x are independently an integer from 3 to 200;
A is a tumor antigen; and
F is a polysaccharide having a molecular weight of about 10,000 to about 1,000,000 Daltons and is connected to L³ via an ether group,
wherein the TLR9 agonist comprises a polynucleotide comprising the sequence 5′-TCGNs-3′, wherein s=4 to 47 and each N is a nucleoside, and wherein one or more linkages between the nucleotides and between the 3′-terminal nucleotide and L¹ are phosphorothioate ester linkages, and
wherein A is a tumor antigen comprising a polypeptide of about 9 to about 1000 amino acids and comprises at least one thiol group,
wherein the method comprises:
reacting a composition comprising compounds of the formula D-L^{1a}-SH, where D is independently as defined for formula (I) and L^{1a} is (CH₂)ₘ where m is independently an integer from 2 to 9, and reacting a composition comprising compounds of the formula A, with a composition comprising a distribution of compounds of formula (II):

[L^{2a}-(PEG)-L³]_{y}-F (II)
wherein L³, PEG and F are as defined for formula (I);
L^{2a} is

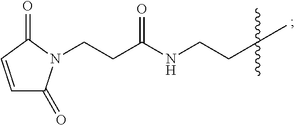 [SMILES: *CCNC(=O)CCN1C(=O)C=CC1=O]

and
y is an integer from 6 to 350; provided that no less than the sum oft and x (y>=t+x).

In some embodiments, the F moieties in the composition comprising compounds of formula (II) have an average molecular weight between about 200,000 and about 600,000 in Daltons, and wherein the compounds of formula (II) in the composition have an average loading ratio (y) between about 60 and about 250. In some embodiments, the F moieties in the composition comprising compounds of formula (II) have an average molecular weight of about 400,000±100,000 Daltons. In some embodiments, the compounds of formula (II) in the composition have an average loading ratio (y) of about 120±30, about 150±30, about 185±30 or about 190±30. The average loading ratio for the TLR9 agonist (x) and average loading ration of the tumor antigen (t) in the co-conjugate composition may be the same or different. In some embodiments, the co-conjugate compounds of formula (I) in the composition have an average loading ratio for the TLR9 agonist (x) of about 10 to about 120, about 10 to about 100, about 10 to about 80, about 10 to about 60, about 20 to about 40, or about 30±10, and/or an average loading ratio for the tumor antigen (t) of about 10 to about 120, about 10 to about 100, about 20 to about 100, about 25 to about 100, about 35 to about 75, or about 55±20. Loading ratios for the FICOLL derivatives are on a molar basis.

In some embodiments, the method for preparing a composition comprising compounds of formula (I) further comprises:
reacting a compound of the formula L^{2a}-(PEG)-L^{3a}-Lv, where
   L^{2a} is a moiety comprising a maleimide group,
   PEG is a polyethylene glycol (e.g., —(OCH₂CH₂)ₙ—, where n=2 to 80),
   L^{3a} is —NHC(O)CH₂CH₂C(O)—, and
   Lv is a leaving group (e.g., (2,5-dioxopyrrolidin-1-yl)oxy),
with a composition comprising compounds of the formula (III):

[NH₂CH₂CH₂NHC(O)CH₂]_{z}-F (III)
wherein F is independently a branched copolymer of sucrose and epichlorohydrin and is connected to L³ via an ether group, and
z is independently an integer from 6 to 400;
and wherein the F moieties in the composition comprising compounds of formula (III) have an average molecular weight between about 200,000 and about 600,000 Daltons, and the compounds of the formula (III) have an average loading ratio (z) between about 60 and about 280;

to form a composition comprising compounds of the formula (II):

[L^{2a}-(PEG)-L³]_{y}-F (II)
wherein y is independently an integer from 6 to 350.

In some embodiments, the F moieties in the composition comprising compounds of formula (III) have an average molecular weight between about 300,000 and about 500,000 in Daltons. In some embodiments, the F moieties have an average molecular weight of about 400,000±100,000 Daltons. In some embodiments, the compounds of the formula (III) have an average loading ratio (z) between about 50 and about 350, between about 50 and about 280, between about 60 and about 250, between about 60 and about 180, between about 60 and about 150, between about 90 and about 280, between about 90 and about 250, between about 90 and about 200, between about 90 and about 150, between about 120 and about 280, between about 120 and about 250, between about 150 and about 280, between about 150 and about 250, between about 180 and about 280, between about 180 and about 250, between about 200 and about 250 or between about 210 and about 230. In some embodiments, the compounds of the formula (III) have an average loading ratio (z) of about 120±30, about 150±30, about 180±30, about 220±30 or about 220±20. In some embodiments, the composition comprising compounds of formula (III) is AECM FICOLL® 400.

In some embodiments, the methods of preparing a compound of formula (I) or a composition comprising compounds of formula (I) further comprise purifying the compounds of the formula (I), and/or any of the intermediate compounds such as compounds of formula (II) and compounds of formula (III). In some embodiments, the method further comprises purifying the compounds of formula (I) by diafiltration. In some embodiments, the method further comprises purifying the compounds of formula (I) by diafiltration using a 100,000 molecular weight cut off (MWCO) membrane.

Further provided is a composition or a particle prepared using a method described herein, for an immunogenic composition comprising a particle comprising a TLR9 agonist and a tumor antigen each associated with a biocompatible multimerization agent using any of the methods detailed herein.

Particle size of the particles detailed herein are measured using methods known in the art and described herein, for example, dynamic light scattering (DLS) is can be used to determine the particle size range and a mean particle size. A Flow Cam (Particle Characterization Lab, Novato, Calif.) method can also be used to determine the mean diameter and size distribution of the particles.

Molecular weight of the TLR9 agonist-polysaccharide-tumor antigen co-conjugate polymers can be measured using methods know in the art, for example, hydrodynamic methods based on viscosity and methods based on light-scattering.

### III. Pharmaceutical Compositions

Some immunogenic compositions of the present disclosure are pharmaceutical compositions comprising particles and a pharmaceutically acceptable excipient. Pharmaceutical compositions of the present disclosure may be in the form of a solution or a suspension. Alternatively, the pharmaceutical compositions may be a dehydrated solid (e.g., freeze dried or spray dried solid). The pharmaceutical compositions of the present disclosure are preferably sterile, and preferably essentially endotoxin-free. The term “pharmaceutical composition” is used interchangeably herein with the terms “medicinal product” and “medicament.”

### A. Excipients

Pharmaceutically acceptable excipients of the present disclosure include for instance, solvents, bulking agents, buffering agents, tonicity adjusting agents, and preservatives. See, e.g., Pramanick et al., Pharma Times, 45:65-77, 2013. In some embodiments the pharmaceutical compositions may comprise an excipient that functions as one or more of a solvent, a bulking agent, a buffering agent, and a tonicity adjusting agent (e.g., sodium chloride in saline may serve as both an aqueous vehicle and a tonicity adjusting agent). The pharmaceutical compositions of the present disclosure are suitable for parenteral administration. That is the pharmaceutical compositions of the present disclosure are not intended for enteral administration.

In some embodiments, the pharmaceutical compositions comprise an aqueous vehicle as a solvent. Suitable vehicles include for instance sterile water, saline solution, phosphate buffered saline, and Ringer's solution. In some embodiments, the composition is isotonic.

The pharmaceutical compositions may comprise a bulking agent. Bulking agents are particularly useful when the pharmaceutical composition is to be lyophilized before administration. In some embodiments, the bulking agent is a protectant that aids in the stabilization and prevention of degradation of the active agents during freeze or spray drying and/or during storage. Suitable bulking agents are sugars (mono-, di- and polysaccharides) such as sucrose, lactose, trehalose, mannitol, sorbital, glucose and raffinose.

The pharmaceutical compositions may comprise a buffering agent. Buffering agents control pH to inhibit degradation of the active agent during processing, storage and optionally reconstitution. Suitable buffers include for instance salts comprising acetate, citrate, phosphate or sulfate. Other suitable buffers include for instance amino acids such as arginine, glycine, histidine, and lysine. The buffering agent may further comprise hydrochloric acid or sodium hydroxide. In some embodiments, the buffering agent maintains the pH of the composition within a range of 6 to 9. In some embodiments, the pH is greater than (lower limit) 6, 7 or 8. In some embodiments, the pH is less than (upper limit) 9, 8, or 7. That is, the pH is in the range of from about 6 to 9 in which the lower limit is less than the upper limit.

The pharmaceutical compositions may comprise a tonicity adjusting agent. Suitable tonicity adjusting agents include for instance dextrose, glycerol, sodium chloride, glycerin and mannitol.

The pharmaceutical compositions may comprise a preservative. Suitable preservatives include for instance antioxidants and antimicrobial agents. However, in preferred embodiments, the pharmaceutical composition is prepared under sterile conditions and is in a single use container, and thus does not necessitate inclusion of a preservative.

### B. Kits

Additionally, the present disclosure provides kits that comprise an immunogenic composition such as a pharmaceutical composition and a set of instructions relating to the use of the composition for the methods describe herein. The pharmaceutical composition of the kits is packaged appropriately. For example, if the pharmaceutical composition is a freeze-dried power, a vial with a resilient stopper is normally used as the container-closure system so that the powder may be easily resuspended by injecting fluid through the resilient stopper. If the pharmaceutical composition is a liquid, a silicon dioxide vial (e.g., SCHOTT Type I Plus®) with a rubber stopper (e.g., Exxpro halobutyl elastomer) and an aluminum crimp-top is normally used as the container-closure system. In certain embodiments, the kit contains a pharmaceutical composition that is comprised of a two vial container-closure system in order to facilitate dose and schedule flexibility during clinical trials, where one vial contains the tumor antigen(s) adsorbed to the multimerization agent (e.g., aluminum hydroxide particle), the second vial contains the TLR9 agonist (e.g., D64-04), and prescribed volumes of the two solutions are mixed prior to administration. In other preferred embodiments, the kit contains a pharmaceutical composition that is comprised of a two vial container-closure system in order to facilitate use of tumor neoantigen(s) in a “personalized medicine” approach, where one vial contains the TLR9 agonist (e.g., D64-04) adsorbed to the multimerization agent (e.g., aluminum hydroxide particle), the second vial contains a solution with one or more tumor neoantigens, and prescribed volumes of the two solutions are mixed prior to administration. Tumor neoantigens are typically identified by sequencing a patient's tumor genome.

In some embodiments, the kits further comprise a device for administration (e.g., syringe and needle) of the pharmaceutical composition. In other embodiments, the kits further comprise a pre-filled syringe/needle system, autoinjectors, or needleless devices. The instructions relating to the use of the pharmaceutical composition generally include information as to dosage, schedule and route of administration for the intended methods of use.

### IV. Methods of Use

The pharmaceutical compositions of the present disclosure are suitable for treating cancer in a mammalian subject in need thereof. Mammalian subjects include but are not limited to humans, nonhuman primates, rodents, pets, and farm animals. In some embodiments, the pharmaceutical compositions may be administered to the subject in an amount effective to achieve a specific outcome.

### A. Dosage and Mode of Administration

As with all pharmaceutical compositions, the effective amount and mode of administration may vary based on several factors evident to one skilled in the art. An important factor to be considered is whether the pharmaceutical composition is to be administered as a stand-alone treatment, or as part of a combination of therapeutic agents. Other factors to be considered include the outcome to be achieved, and the number of doses to be administered.

A suitable dosage range is one that provides the desired effect. Dosage may be determined by the amount of TLR9 agonist comprising a polynucleotide to be administered to the subject. An exemplary dosage range of the polynucleotide given in amount to be delivered by subject weight is from about 5 to 5000 mcg/kg. In some embodiments, the dosage is greater than about (lower limit) 5, 10, 50, 100, 150, 200, 250, 300, 350, 400, 450, 500, 750 or 1000 mcg/kg. In some embodiments, the dosage is less than about (upper limit) 5000, 4000, 3000, 2000, 1000, 750, 500, 450, 400, 350, 300, 250, 200, 150, or 100 mcg/kg. That is, the dosage is anywhere in the range of from about 5 to 5000 mcg/kg in which the lower limit is less than the upper limit. An exemplary dosage range of the polynucleotide given in amount to be delivered to a subject is from about 100 mcg to about 100 mg. In some embodiments, the dosage is greater than about (lower limit) 100, 250, 500, 750, 1000, 1500, 2000, 2500, 3000, 3500, 4000, 4500, or 5000 mcg. In some embodiments, the dosage is less than about (upper limit) 100, 75, 50, 25, 20, 15, or 10 mg. That is, the dosage is anywhere in the range of from about 100 to 100,000 mcg in which the lower limit is less than the upper limit.

Dosage may also be determined by the amount of antigen (e.g., peptide antigen(s)) to be administered to the subject. An exemplary dosage range given in amount to be delivered to a subject is from about 1 mcg to 50 mcg. In some embodiments, the antigen dosage is greater than about (lower limit) 1, 5, 10, 15, 20, 25, 30, 35, or 40, 50, 100, 250, 500, 750 or 1000 mcg. In some embodiments, the antigen dosage is less than about (upper limit) 1000, 750, 500, 250, 100, 50, 45, 40, 35, 30, 25, 20, 15, or 10 mcg. That is, the antigen dosage of each antigen is anywhere in the range of from about 1 to 1000 mcg in which the lower limit is less than the upper limit. In further embodiments, the dosage range given in amount to be delivered to the subject is from 1 mcg to 1000 mcg of each antigen. In such embodiments, the antigen dosage is greater than about (lower limit) 1, 5, 10, 15, 20, 25, 30, 35, 40, 50, 100, 250, 500 or 750 mcg. In such embodiments, the antigen dosage is less than about (upper limit) 1000, 750, 500, 250, 100, 50, 45, 40, 35, 30, 25, 20, 15, or 10 mcg. That is, the antigen dosage of each antigen is anywhere in the range of from about 1 to 1000 mcg in which the lower limit is less than the upper limit.

In some embodiments, the pharmaceutical compositions of the present disclosure are intended for parenteral administration (e.g., not oral or rectal administration). Suitable routes of administration include injection, topical, and inhalation. In particular, the pharmaceutical compositions of the present disclosure may be administered by a route such as intravenous, intramuscular, subcutaneous, epidermal (gene gun), transdermal, and inhalation. However, the preferred route of administration is by intratumoral delivery.

A suitable dosing regimen is one that provides the desired effect in a prophylactic or therapeutic context. The number of doses administered by a chosen route may be one or more than one. Frequency of dosing may range from every two, three, four, five or six days, weekly, bi-weekly, monthly, bi-monthly, or 3 to 12 months between doses. In some embodiments, 2 doses are administered with the second dose being administered one to two months after the first dose. In some embodiments, 3 doses are administered with the second dose being administered one to two months after the first dose, and the third dose being administered one to five months after the second dose. In other embodiments, 3, or 4 doses may be administered on a bi-weekly or monthly basis. In other embodiments, a shorter or longer period of time may elapse in between doses. In certain embodiments, the interval between successive dosages may vary in terms of number of weeks or number of months. In one embodiment, a series of 2, 3, 4, 5, or 6 weekly (or more frequent) doses may be administered followed by a second series of a number of weekly (or more frequent) doses at a later time point. One of skill in the art will be able to adjust the dosage regimen by measuring biological outcomes as exemplified in the examples, such as antigen-specific antibody responses or tumor regression.

### B. Stimulation of an Immune Response

In brief, the present disclosure provides methods of stimulating an immune response in a mammalian subject, comprising administering to a mammalian subject a pharmaceutical composition in an amount sufficient to stimulate an immune response in the mammalian subject. “Stimulating” an immune response, means increasing the immune response, which can arise from eliciting a de novo immune response (e.g., as a consequence of an initial vaccination regimen) or enhancing an existing immune response (e.g., as a consequence of a booster vaccination regimen). In some embodiments, stimulating an immune response comprises one or more of the group consisting of: stimulating cytokine production; stimulating B lymphocyte proliferation; stimulating interferon pathway-associated gene expression; stimulating chemoattractant-associated gene expression; and stimulating plasmacytoid dendritic cell (pDC) maturation. Methods for measuring stimulation of an immune response are known in the art and described in the biological examples of the present disclosure.

For instance, the present disclosure provides methods of inducing an antigen-specific antibody response in a mammalian subject by administering to a mammalian subject the pharmaceutical composition in an amount sufficient to induce an antigen-specific antibody response in the mammalian subject. “Inducing” an antigen-specific antibody response means increasing titer of the antigen-specific antibodies above a threshold level such as a pre-administration baseline titer or a seroprotective level.

Analysis (both qualitative and quantitative) of the immune response can be by any method known in the art, including, but not limited to, measuring antigen-specific antibody production (including measuring specific antibody subclasses), activation of specific populations of lymphocytes such as B cells and helper T cells, production of cytokines such as IFN-alpha, IFN-gamma, IL-6, IL-12 and/or release of histamine. Methods for measuring antigen-specific antibody responses include enzyme-linked immunosorbent assay (ELISA). Activation of specific populations of lymphocytes can be measured by proliferation assays, and with fluorescence-activated cell sorting (FACS). Production of cytokines can also be measured by ELISA.

Preferably, a Th1-type immune response is stimulated (i.e., elicited or enhanced). With reference to present disclosure, stimulating a Th1-type immune response can be determined in vitro or ex vivo by measuring cytokine production from cells treated with an active agent of the present disclosure (polynucleotide TLR9 agonist) as compared to control cells not treated with the active agent. Examples of “Th1-type cytokines” include, but are not limited to, IL-2, IL-12, IFN-gamma and IFN-alpha. In contrast, “Th2-type cytokines” include, but are not limited to, IL-4, IL-5, and IL-13. Cells useful for the determination of immunostimulatory activity include cells of the immune system, such as antigen presenting cells lymphocytes, preferably macrophages and T cells. Suitable immune cells include primary cells such as peripheral blood mononuclear cells, including plasmacytoid dendritic cells and B cells, or splenocytes isolated from a mammalian subject.

Stimulating a Th1-type immune response can also be determined in a mammalian subject treated with an active agent of the present disclosure (polynucleotide TLR9 agonist) by measuring levels of IL-2, IL-12, and interferon before and after administration or as compared to a control subject not treated with the active agent. Stimulating a Th1-type immune response can also be determined by measuring the ratio of Th1-type to Th2-type antibody titers. “Th1-type” antibodies include human IgG1 and IgG3, and murine IgG2a. In contrast, “Th2-type” antibodies include human IgG2, IgG4 and IgE and murine IgG1 and IgE.

### C Treating Cancer

The present disclosure provides methods of treating cancer in a mammalian subject, comprising administering to a mammalian subject an immunogenic composition comprising particles of the present disclosure in an amount sufficient to treat cancer in the mammalian subject. “Treating” cancer means to bring about a beneficial clinical result such as causing remission or otherwise prolonging survival as compared to expected survival in the absence of treatment. In some embodiments, “treating” cancer comprises shrinking the size of a tumor or otherwise reducing viable cancer cell numbers. In other embodiments, “treating” cancer comprises delaying growth of a tumor. In some preferred embodiments, the present disclosure provides methods of treating cancer in a mammalian subject in need thereof, comprising administering to the subject an effective amount of an immunogenic composition comprising particles of the present disclosure by intratumoral delivery.

In some preferred embodiments, “treating cancer” comprises assessing a patient's response to the immunogenic composition according to the Response Evaluation Criteria in Solid Tumors (RECIST version 1.1) as described (see, e.g., Eisenhauer et al., 2009 Eur J Cancer, 45:228-247). Response criteria to determine objective anti-tumor responses per RECIST include: complete response; partial response; progressive disease; and stable disease.

### EXAMPLES

Abbreviations: Ab (antibody); Ag (antigen); Alum (aluminum salt adjuvant such ALHYDROGEL® 85 marketed by Brenntag Nordic A/S); Al(OH)₃ (aluminum hydroxide); AlPO₄ (aluminum phosphate); BMDC (bone marrow-derived dendritic cell); CC (chimeric compound); CpG (polynucleotide including an unmethylated CG dinucleotide or a chimeric compound thereof); CTAG1 (cancer/testis antigen 1); CTRL (control); DC (dendritic cell); ELISA (enzyme-linked immunosorbent assay); EC₅₀ (half maximal effective concentration); FACS (fluorescence-activated cell sorting); FCS (fetal calf serum); Fic (a high MW, branched copolymer of sucrose and epichlorohydrin such as FICOLL® marketed by GE Healthcare); HEG (hexaethylene glycol); HLA (human leukocyte antigen); Hypb (hydrophobicity); IFN-γ (interferon-gamma); IPA (isopropanol); IT (intratumoral); mcg or μg (microgram); MAGEA (melanoma antigen, family A); MW (molecular weight); MWCO (molecular weight cut off); NaCl (sodium chloride); NaOAc (sodium acetate); NY-ESO-1 or NYESO1 (New York esophageal squamous cell carcinoma 1); Nu (nucleic acid moiety); ODN (oligodeoxynucleotide); OLP (overlapping long peptide); OVA (ovalbumin); PADRE (PAn HLA DR-binding Epitope); PBMC (peripheral blood mononuclear cell); PBS (phosphate buffered saline); PEG (polyethylene glycol); PN (polynucleotide); SC (subcutaneous); SCC (squamous cell carcinoma); SEM (standard error of the mean); Sp (non-nucleic acid spacer moiety); TFF (tangential flow filtration); TIL (tumor infiltrating leukocytes); TLR9 (Toll-like receptor 9); TNF-α (tumor necrosis factor-alpha); and WT (wild type).

Although, the present disclosure has been described in some detail by way of illustration and example for purposes of clarity and understanding, it will be apparent to those skilled in the art that certain changes and modifications may be practiced. Therefore, the following synthetic and biological examples should not be construed as limiting the scope of the present disclosure, which is delineated by the appended claims.

### Example S1: Structure of Polynucleotides and Chimeric Compounds

Table S1-1 shows the structures of polynucleotides and chimeric compounds, which are generally referred to herein interchangeably as CpGs or CpG-ODNs. The nucleotides in the polynucleotides and chimeric compounds are 2′-deoxyribopolynucleotides. HEG is a hexaethylene glycol spacer moiety and was incorporated using 18-O-dimethoxytritylhexaethyleneglycol, 1-((2-cyanoethyl)-(N,N-isopropyl))-phosphoramidite. All internucleotide linkages and linkages between nucleic acid moieties and spacer moieties are phosphorothioate ester linkages.

TABLE S1-1
Polynucleotide (PN) and Chimeric Compound (CC) Structures\hat{ }

| | SEQ | |
|---|---|---|
| | ID | |
| Cmpd. | NO: | Sequence |
| — | 1 | 5′-TCGNs-3′ |
| | | wherein each N is an independently selected |
| | | nucleoside, and s = 4 to 47 |
| — | — | Nu1-Sp1-Nu2-Sp2-Nu3 |
| | | wherein Nu1, Nu2 and Nu3 are nucleic acid moieties, |
| | | Sp1 and Sp2 are non nucleic acid spacer moieties, and |
| | | Nu1 consists of the sequence 5′-TCGNs-3′ where s = 4 |
| | | to 47 |
| — | 2 | 5′-(TCG(Nq))iNw(X1X2CGX2′X1′ (CG)p)j, Nv-3′ |
| | | wherein each N is an independently selected |
| | | nucleoside, p = 0 or 1, q = 0, 1, 2, 3, 4 or 5, v = 0 |
| | | to 41, w = 0, 1 or 2, i = 1, 2, 3 or 4, j = 1, 2, 3 or |
| | | 4, X1 and X1′ are self-complementary, X2 and X2′ are |
| | | self-complementary |
| — | 3 | 5′-TCGNq(X1X2CGX2′X1′CG)jNv-3′ |
| | | wherein each N is an independently selected |
| | | nucleoside, q = 0, 1, 2, 3, 4 or 5, v = 1 to 39, j = |
| | | 1, 2, 3 or 4, X1 and X1′ are self-complementary, X2 |
| | | and X2′ are self-complementary |
| — | 4 | 5′-TCGNqAACGTTCGAACGTTCGAANr-3′ |
| | | wherein q = 0, 1, 2, 3, 4 or 5, r = 0 to 29 |
| D61-01 | 5 | 5′-TCGGCGC-3′-HEG-5′-AACGTTC-3′-HEG-5′-TCGGCGC-3′ |
| D61-02 | 5 | 5′-TCGGCGC-3′-HEG-5′-AACGTTC-3′-HEG-5′-TCGGCGC-3′- |
| | | (CH2)6-SS-(CH2)6-OH [see Example S9, (D61-01)-3′-SS)] |
| D61-03 | 5 | 5′-TCGGCGC-3′-HEG-5′-AACGTTC-3′-HEG-5′-TCGGCGC-3′- |
| | | (CH2)6-SH [see Example S9, (D61-01)-3′-SH)] |
| D61-04 | 6 | 5′-TCG AAC GTT CGA ACG TTC GAA CGT TCG AAT-3′ |
| D61-05 | 7 | 5′-TCG TTC GAA CGT TCG AAC GTT CGA A-3′ |
| D61-06 | 8 | 5′-TCG AAC GTT CGA ACG TTC GAA TTT T-3′ |
| D61-07 | 9 | 5′-TCG TAA CGT TCG AAC GTT CGA ACG TTA-3′ |
| D61-08 | 10 | 5′-TCG TAA CGT TCG AAC GTT CGA AC-3′ |
| D61-09 | 72 | 5′-TCGCCGG-3′-HEG-5′-AACGTTC-3′-HEG-5′-TCGCCGG-3′ |
| — | 73 | 5′-AACGTTNm-3′, where m = 1 to 44 |

\hat{ }Unless otherwise noted, the polynucleotides and chimeric compounds of SEQ ID NOs: 5, 6, 7, 8, 9, 10 and 27 include 2′-deoxy-ribopolynucleotides and the internucleotide linkages are phosphorothioate ester linkages. Different compounds are given the same SEQ ID NO when the only difference is in non-nucleic acid moieties.

Table S1-1 also shows CCs (e.g., D61-02, D61-03) with an end linking group (e.g., —(CH2)6-SS—(CH2)6-OH, —(CH2)6-SH) used to covalently link these molecules with a branched carrier moiety (e.g., [Maleimide-PEGn]y-FICOLL) to create branched CCs (see Example S9). These linking groups are connected to the polynucleotide or CC via a spacer moiety with a phosphorothioate linkage. The 3′-C6-disulfide linker was incorporated using 1-O-dimethoxytrityl-hexyl-disulfide, 1′-succinyl-solid support. The polynucleotides and chimeric compounds were manufactured by TriLink Biotechnologies (San Diego, Calif., USA) or Nitto Denko Avecia (Milford, Mass., USA) and were received as freeze-dried solids.

### Example S2: Structure of Polypeptide Antigens

Table S2-1 shows the primary structure of polypeptide antigens, which are referred to herein interchangeably as polypeptides or peptides. The polypeptides were purchased from either Bio-Synthesis Inc. (Lewisville, Tex., USA) or C S Bio (Menlo Park, Calif., USA).

TABLE S2-1
Polypeptide Structures

| SEQ | | | |
|---|---|---|---|
| ID | | | |
| NO: | Description | Sequence | Hypb\hat{ } |
| 11 | OVApep | CSGLEQLESIINFEKLTEWTSSNVMEERKIKV | 38% |
| 12 | OVA class I | SIINFEKL | 50% |
| 13 | OVA class II | TEWTSSNVMEERKIKV | 27% |
| 14 | Triple | PEG₂₄-VGALEGPRNQDWLAKXVAAWTLKAAATAYRYHLLSSVY | 60% |
| | | DFFVWLSC wherein X = L-cyclohexylalanine | |
| 15 | Trp1 | TAYRYHLL | 63% |
| 16 | Trp2 | SVYDFFVWL | 78% |
| 17 | gp100 | EGPRNQDWL | 22% |
| 18 | PADRE | AKXVAAWTLKAAA | 75% |
| | | wherein X = L-cyclohexylalanine | |
| 19 | HPV16 | EVYDFAFRDLQAEPDRAHYNIVTFCCKC | 54% |
| 20 | HPV16-E6 | EVYDFAFRDL | 60% |
| 21 | HPV16-E7 | QAEPDRAHYNIVTF | 43% |
| 22 | Neo | PFPAAVILRDALHWNDLAVIPAGVVHNEFVDWENVSPELNST | 50% |
| 23 | Pbkm | PFPAAVILRDAL | 67% |
| 24 | Def8m | HWNDLAVIPAGVVHN | 53% |
| 25 | Kif8m | EFVDWENVSPELNST | 33% |
| 26 | AH1 CII | VTYHSPSYVYHQFERRAKLVQFIKDRISVVQASC | 47% |
| 27 | B16-F10-PEG | PEG₂₄-AAVILRDALHWNDLAVIPAGVVHNEFVDWENVSPELNST | 51% |
| 28 | Triple AAY | VGALEGPRNQDWLAAYTAYRYHLLAAYSVYDFFVWLAAYALXVAA | 71% |
| | | WTLKAAA wherein X = L-cyclohexylalanine | |
| 29 | TRP1 OLP | AKXVAAWTLKAAAAAYTAYRYHLL | 74% |
| | | wherein X = L-cyclohexylalanine | |
| 30 | GP100 OLP | AVGALEGPRNQDWLGVPRQL | 40% |
| 31 | TRP3 OLP | QIANCSVYDFFVWLHYYSV | 68% |
| 32 | HPV-16-PEG | PEG₂₄-EVYNFAFRDLQAEPDRAHYNIVTFCCKC | 54% |
| 33 | DEF8 OLP | SHCHWNDLAVIPAGVVHNWDFEPRKVS | 44% |
| 34 | Kif18b OLP | PSKPSFQEFVDWENVSPELNSTDQPFL | 30% |
| 35 | Pbk OLP | DSGSPFPAAVILRDALHMARGLKYLHQ | 48% |
| 36 | AH1 AAY | VTYHSPSYVYHQFERRAKAAYLVQFIKDRISVVQASC | 51% |
| 37 | AH1-PEG | PEG₂₄-VTYHSPSYVYHQFERRAKLVQFIKDRISVVQASC | 47% |
| 38 | TYR₁₄₆₋₁₅₆ | SSDYVIPIGTY | |
| 39 | TYR_{240-251(C244S)} | DAEKSDICTDEY | |
| 40 | TYR_{369-377(N371D)} | YMDGTMSQV | |
| 41 | gp100₁₇₋₂₅ | ALLAVGATK | |
| 42 | gp100_{209-217(T210M)} | IMDQVPFSV | |
| 43 | gp100_{198-227(1210M)} OLP | VPLAHSSSAFTIMDQVPFSVSVSQLRALDG | |
| 44 | gp100₂₈₀₋₂₈₈ | YLEPGPVTA | |
| 45 | gp100₆₁₄₋₆₂₂ | LIYRRRLMK | |
| 46 | MAGE-A1₉₆₋₁₀₄ | SLFRAVITK | |
| 47 | MAGE-A1₁₆₁₋₁₆₉ | EADPTGHSY | |
| 48 | MAGE-A3₁₆₈₋₁₇₆ | EVDPIGHLY | |
| 49 | MAGE-A10₂₅₄₋₂₆₂ | GLYDGMEHL | |
| 50 | MAGE-A10₂₄₅₋₂₇₄ OLP | VIWEALNMMGLYDGMEHLIYGEPRKLLTQD | |
| 51 | TT₈₃₀₋₈₄₄ | AQYIKANSKFIGITEL | |
| 74 | MELAN A₁₆₋₄₀ | GHGHSYTTAEELAGIGILTVILGVL | 48% |

\hat{ }Hydrophobicity (Hypb) was determined using an online peptide properties calculator found at “www.biosyn.com/peptidepropertycalculatorlanding.aspx” and is expressed as a percentage of the full length amino acid sequence. (polyethylene glycol)₂₄/PEG₂₄ and cyclohexylalanine were not included in the Hypb calculations.

The OVApep antigen includes an ovalbumin (OVA) class I epitope plus seven N-terminal amino acids from OVA to facilitate peptide excision (Cascio et al., 2001 EMBO J, 20:2357-2366) and an OVA class II epitope (Maecker et al., 1998 J Immunol, 161:6532-6536). A cysteine residue was added to the N-terminus for conjugation purposes.

The Triple antigen includes three class I restricted melanoma epitopes (gp100, Trp1, and Trp2) and an artificial Pan class II DR restricted Epitope (PADRE) as a fusion polypeptide, plus a PEG₂₄ group. The melanoma epitopes (gp100, van Stipdonk et al., 2009 Cancer Res, 69:7784-7792; Trp1, Dyall et al., 1998 J Exp Med, 188:1553-1561; and Trp2, Liu et al., 2003 J Immunother, 26:301-312) and the artificial epitope (Alexander et al., 1994 Immunity, 1:751-761) have been described previously. The PEG₂₄ group was attached to the N-terminus via an amide linkage in order to reduce hydrophobicity with the goal of aiding solubility.

Additionally, the peptides were modified to include a single cysteine residue at either the N- or C-terminus to enable covalent linkage to a maleimide-functionalized polysaccharide via the thiol group of the cysteine. The cysteine in the peptide may also be present for non-covalent adsorption to an aluminum salt complex, but is not required.

Table S2-2 shows the primary structure of several New York Esophageal Squamous Cell Carcinoma 1 (NY-ESO-1 or NYESO1) polypeptide antigens. NY-ESO-1 is also known as cancer/testis antigen 1 (CTAG1). Some antigens of Table S2-2 are overlapping long peptide (OLP) antigens comprising amino acid sequences of more than one epitope of the CTAG1 protein (UniProtKB database accession number P78358, set forth as SEQ ID NO:60). Several NY-ESO-1 peptide antigens listed below have been employed in human clinical trials of candidate melanoma vaccines. CTAG1 is highly expressed in poor-prognosis melanomas and the NY-ESO-1 peptide antigens promoted efficient MHC presentation of minimal epitope sequences by antigen presenting cells (see Slingluff, 2011 Cancer J 17:343; Tsuji et al., 2013 Cancer Immunol Res 1:340; Wada et al., 2014 J Immunother 37:84; Sabbatini et al., 2012 Clin Cancer Res 18:6497).

Peptide antigens of SEQ ID NOs:55-58 were chemically synthesized using solid phase peptide synthesis methods, then purified and analytically characterized using known techniques (see Behrendt et al., 2016 J Pept Sci, 22:4). The peptide antigens of SEQ ID NOs:55-58 were purchased from either Bio-Synthesis Inc. (Lewisville, Tex., USA) or C S Bio (Menlo Park, Calif., USA). The 94 amino acid polypeptide of SEQ ID NO:59 was expressed using conventional recombinant mammalian expression methods, then purified and analytically characterized using known techniques (see Fischer et al., 2015 Biotechnol Adv, 33:1878).

TABLE S2-2
NY-ESO-1 Structures

| SEQ | | | |
|---|---|---|---|
| ID | | | |
| NO: | Description | Sequence | Hypb\hat{ } |
| 52 | NY-ESO-1₅₃₋₆₂ | ASGPGGGAPR | |
| 53 | NY-ESO-1₉₄₋₁₀₂ | MPFATPMEA | |
| 54 | NY-ESO-1₁₅₇₋₁₆₅ | SLLMWITQC | |
| 55 | NY-ESO-1₇₉₋₁₀₈ OLP | GARGPESRLLEFYLAMPFATPMEAELARRS | 47% |
| 56 | NY-ESO-1₁₀₀₋₁₂₉ OLP | MEAELARRSLAQDAPPLPVPGVLLKEFTVS | 47% |
| 57 | NY-ESO-1₁₂₁₋₁₅₀ OLP | VLLKEFTVSGNILTIRLTAADHRQLQLSIS | 47% |
| 58 | NY-ESO-1₁₄₂₋₁₇₃ OLP | HRQLQLSISSCLQQLSLLMWITQCFLPVFLAQ | 56% |
| 59 | NY-ESO-1₇₉₋₁₇₃ OLP | GARGPESRLLEFYLAMPFATPMEAELARRSLAQDAPPLPVPGV | 51% |
| | | LLKEFTVSGNILTIRLTAADHRQLQLSISSCLQQLSLLMWITQ | |
| | | CFLPVFLAQ | |
| 60 | CTAG1 | MQAEGRGTGG STGDADGPGG PGIPDGPGGN AGGPGEAGAT | |
| | | GGRGPRGAGA ARASGPGGGA PRGPHGGAAS GLNGCCRCGA | |
| | | RGPESRLLEF YLAMPFATPM EAELARRSLA QDAPPLPVPG | |
| | | VLLKEFTVSG NILTIRLTAA DHRQLQLSIS SCLQQLSLLM | |
| | | WITQCFLPVF LAQPPSGQRR | |

\hat{ }Hydrophobicity (Hypb) was determined using an peptide properties calculator found at “www.biosyn.com/peptidepropertycalculatorlanding.aspx” and is expressed as a percentage of the hydrophobic amino acids in the full length amino acid sequence.

Table S2-3 shows the primary structure of several melanoma antigen family A proteins (MAGEA), as well as the amino acid sequence of several polypeptides that contain one or more minimal 9 amino acid epitopes from the corresponding MAGEA protein. The MAGEA proteins are promising immunotherapy targets due to their low expression in non-malignant tissues and high levels of expression in various tumors, including cutaneous squamous cell carcinomas (SCC), esophageal SCC, head and neck SCC, cervical/anal SCC, lung SCC, adenocarcinomas, bladder urothelial carcinomas, ovarian carcinomas, endometrial carcinomas, lung small cell carcinomas, breast mucinous carcinomas, hepatocellular carcinomas, thymic carcinomas and mesotheliomas (see e.g., Kerkar et al., 2016 J Immunother, 39:181; Park et al., 2016 J Immunother 39:1). Several MAGEA proteins have been employed as antigens in human clinical trials of candidate melanoma vaccines, including MAGEA3 and MAGEA10 (see e.g., Vansteenkiste et al., 2016 Lancet Oncol 16:822; ClinicalTrials.gov Identifier NCT02989064). The full-length MAGEA proteins of SEQ ID NOs: 61-71 are expressed using conventional recombinant mammalian expression methods, then purified and characterized using known techniques (see Fischer et al., 2015 Biotechnol Adv, 33:1878). Peptide antigens ranging from about 9 amino acids to about 60 amino acids (e.g., SEQ ID Nos: 46-50) of the full length proteins shown in Table S2-3 (SEQ ID NOs:67-71) are suitable for use as peptide antigens in the compositions and methods of the present disclosure. The peptide antigens are chemically synthesized using solid phase peptide synthesis methods, then purified and characterized using known techniques (see Behrendt et al., 2016 J Pept Sci, 22:4).

TABLE S2-3
MAGEA Structures

| SEQ | | |
|---|---|---|
| ID | | |
| NO: | Antigen | Sequence |
| 61 | MAGEA1 | MSLEQRSLHCKPEEALEAQQEALGLVCVQAATSSSSPLVLGTLEEVPTAGSTDPP |
| | | QSPQGASAFPTTINFTRQRQPSEGSSSREEEGPSTSCILESLFRAVITKKVADLV |
| | | GFLLLKYRAREPVTKAEMLESVIKNYKHCFPEIFGKASESLQLVFGIDVKEADPT |
| | | GHSYVLVTCLGLSYDGLLGDNQIMPKTGFLIIVLVMIAMEGGHAPEEEIWEELSV |
| | | MEVYDGREHSAYGEPRKLLTQDLVQEKYLEYRQVPDSDPARYEFLWGPRALAETS |
| | | YVKVLEYVIKVSARVRFFFPSLREAALREEEEGV |
| 62 | MAGEA2 | MPLEQRSQHCKPEEGLEARGEALGLVGAQAPATEEQQTASSSSTLVEVTLGEVPA |
| | | ADSPSPPHSPQGASSFSTTINYTLWRQSDEGSSNQEEEGPRMFPDLESEFQAAIS |
| | | RKMVELVHFLLLKYRAREPVTKAEMLESVLRNCQDFFPVIFSKASEYLQLVFGIE |
| | | VVEVVPISHLYILVTCLGLSYDGLLGDNQVMPKTGLLIIVLAIIAIEGDCAPEEK |
| | | IWEELSMLEVFEGREDSVFAHPRKLLMQDLVQENYLEYRQVPGSDPACYEFLWGP |
| | | RALIETSYVKVLHHTLKIGGEPHISYPPLHERALREGEE |
| 63 | MAGEA3 | MPLEQRSQHCKPEEGLEARGEALGLVGAQAPATEEQEAASSSSTLVEVTLGEVPA |
| | | AESPDPPQSPQGASSLPTTMNYPLWSQSYEDSSNQEEEGPSTFPDLESEFQAALS |
| | | RKVAELVHFLLLKYRAREPVTKAEMLGSVVGNWQYFFPVIFSKASSSLQLVFGIE |
| | | LMEVDPIGHLYIFATCLGLSYDGLLGDNQIMPKAGLLIIVLAIIAREGDCAPEEK |
| | | IWEELSVLEVFEGREDSILGDPKKLLTQHFVQENYLEYRQVPGSDPACYEFLWGP |
| | | RALVETSYVKVLHHMVKISGGPHISYPPLHEWVLREGEE |
| 64 | MAGEA4 | MSSEQKSQHCKPEEGVEAQEEALGLVGAQAPTTEEQEAAVSSSSPLVPGTLEEVP |
| | | AAESAGPPQSPQGASALPTTISFTCWRQPNEGSSSQEEEGPSTSPDAESLFREAL |
| | | SNKVDELAHFLLRKYRAKELVTKAEMLERVIKNYKRCFPVIFGKASESLKMIFGI |
| | | DVKEVDPTSNTYTLVTCLGLSYDGLLGNNQIFPKTGLLIIVLGTIAMEGDSASEE |
| | | EIWEELGVMGVYDGREHTVYGEPRKLLTQDWVQENYLEYRQVPGSNPARYEFLWG |
| | | PRALAETSYVKVLEHVVRVNARVRIAYPSLREAALLEEEEGV |
| 65 | MAGEA5 | MSLEQKSQHCKPEEGLDTQEEALGLVGVQAATTEEQEAVSSSSPLVPGTLGEVPA |
| | | AGSPGPLKSPQGASAIPTAIDFTLWRQSIKGSSNQEEEGPSTSPDPESVFRAALS |
| | | KKVADLIHFLLLKY |
| 66 | MAGEA6 | MPLEQRSQHCKPEEGLEARGEALGLVGAQAPATEEQEAASSSSTLVEVTLGEVPA |
| | | AESPDPPQSPQGASSLPTTMNYPLWSQSYEDSSNQEEEGPSTFPDLESEFQAALS |
| | | RKVAKLVHFLLLKYRAREPVTKAEMLGSVVGNWQYFFPVIFSKASDSLQLVFGIE |
| | | LMEVDPIGHVYIFATCLGLSYDGLLGDNQIMPKTGFLIIILAIIAKEGDCAPEEK |
| | | IWEELSVLEVFEGREDSIFGDPKKLLTQYFVQENYLEYRQVPGSDPACYEFLWGP |
| | | RALIETSYVKVLHHMVKISGGPRISYPLLHEWALREGEE |
| 67 | MAGEA8 | MLLGQKSQRYKAEEGLQAQGEAPGLMDVQIPTAEEQKAASSSSTLIMGTLEEVTD |
| | | SGSPSPPQSPEGASSSLTVTDSTLWSQSDEGSSSNEEEGPSTSPDPAHLESLFRE |
| | | ALDEKVAELVRFLLRKYQIKEPVTKAEMLESVIKNYKNHFPDIFSKASECMQVIF |
| | | GIDVKEVDPAGHSYILVTCLGLSYDGLLGDDQSTPKTGLLIIVLGMILMEGSRAP |
| | | EEAIWEALSVMGLYDGREHSVYWKLRKLLTQEWVQENYLEYRQAPGSDPVRYEFL |
| | | WGPRALAETSYVKVLEHVVRVNARVRISYPSLHEEALGEEKGV |
| 68 | MAGEA9 | MSLEQRSPHCKPDEDLEAQGEDLGLMGAQEPTGEEEETTSSSDSKEEEVSAAGSS |
| | | SPPQSPQGGASSSISVYYTLWSQFDEGSSSQEEEEPSSSVDPAQLEFMFQEALKL |
| | | KVAELVHFLLHKYRVKEPVTKAEMLESVIKNYKRYFPVIFGKASEFMQVIFGTDV |
| | | KEVDPAGHSYILVTALGLSCDSMLGDGHSMPKAALLIIVLGVILTKDNCAPEEVI |
| | | WEALSVMGVYVGKEHMFYGEPRKLLTQDWVQENYLEYRQVPGSDPAHYEFLWGSK |
| | | AHAETSYEKVINYLVMLNAREPICYPSLYEEVLGEEQEGV |
| 69 | MAGEA10 | MPRAPKRQRCMPEEDLQSQSETQGLEGAQAPLAVEEDASSSTSTSSSFPSSFPSS |
| | | SSSSSSSCYPLIPSTPEEVSADDETPNPPQSAQIACSSPSVVASLPLDQSDEGSS |
| | | SQKEESPSTLQVLPDSESLPRSEIDEKVTDLVQFLLFKYQMKEPITKAEILESVI |
| | | RNYEDHFPLLFSEASECMLLVFGIDVKEVDPTGHSFVLVTSLGLTYDGMLSDVQS |
| | | MPKTGILILILSIVFIEGYCTPEEVIWEALNMMGLYDGMEHLIYGEPRKLLTQDW |
| | | VQENYLEYRQVPGSDPARYEFLWGPRAHAEIRKMSLLKFLAKVNGSDPRSFPLWY |
| | | EEALKDEEERAQDRIATTDDTTAMASASSSATGSFSYPE |
| 70 | MAGEA11 | METQFRRGGLGCSPASIKRKKKREDSGDFGLQVSTMFSEDDFQSTERAPYGPQLQ |
| | | WSQDLPRVQVFREQANLEDRSPRRTQRITGGEQVLWGPITQIFPTVRPADLTRVI |
| | | MPLEQRSQHCKPEEGLQAQEEDLGLVGAQALQAEEQEAAFFSSTLNVGTLEELPA |
| | | AESPSPPQSPQEESFSPTAMDAIFGSLSDEGSGSQEKEGPSTSPDLIDPESFSQD |
| | | ILHDKIIDLVHLLLRKYRVKGLITKAEMLGSVIKNYEDYFPEIFREASVCMQLLF |
| | | GIDVKEVDPTSHSYVLVTSLNLSYDGIQCNEQSMPKSGLLIIVLGVIFMEGNCIP |
| | | EEVMWEVLSIMGVYAGREHFLFGEPKRLLTQNWVQEKYLVYRQVPGTDPACYEFL |
| | | WGPRAHAETSKMKVLEYIANANGRDPTSYPSLYEDALREEGEGV |
| 71 | MAGEA12 | MPLEQRSQHCKPEEGLEAQGEALGLVGAQAPATEEQETASSSSTLVEVTLREVPA |
| | | AESPSPPHSPQGASTLPTTINYTLWSQSDEGSSNEEQEGPSTFPDLETSFQVALS |
| | | RKMAELVHFLLLKYRAREPFTKAEMLGSVIRNFQDFFPVIFSKASEYLQLVFGIE |
| | | VVEVVRIGHLYILVTCLGLSYDGLLGDNQIVPKTGLLIIVLAIIAKEGDCAPEEK |
| | | IWEELSVLEASDGREDSVFAHPRKLLTQDLVQENYLEYRQVPGSDPACYEFLWGP |
| | | RALVETSYVKVLHHLLKISGGPHISYPPLHEWAFREGEE |

### Example S3: Procedure for Adsorption of CpG-ODN to Aluminum Hydroxide

There are two main types of aluminum salts used as adjuvants for vaccines: aluminum hydroxide [Al(OH)₃] and aluminum phosphate [AlPO₄] (Lindblad et al., 2004 Immunol Cell Biol, 82:497-505). At pH 6-8, which is normal during vaccine production, aluminum hydroxide has a positive charge and thus an electrostatic attraction for negatively charged CpG-ODNs and negatively charged proteins and peptides (antigens). Conversely, aluminum phosphate has a negative charge at pH 6-8 and therefor is not suitable for adsorption of a negatively charged CpG-ODN.

Aluminum hydroxide, specifically the aluminum hydroxide formulation marketed as ALHYDROGEL® 85 by Brenntag Nordic A/S (Denmark), manufactured by Brenntag Biosector (Denmark) and purchased from Sergeant Adjuvants (Clifton, N.J., USA), was used in all binding studies. ALHYDROGEL® 85 was supplied as a suspension in purified water at an aluminum concentration of approximately 10±1 mg/ml, and the stated amounts of the examples are based on its aluminum content. ALHYDROGEL® 85 is manufactured under EU GMP Part I for Medicinal Products and is suitable for human use. In preferred embodiments, high loading ratios of CpG and antigen to aluminum hydroxide are employed in order to minimize exposure of mammalian subjects to the aluminum hydroxide salt and aluminum cations.

Analysis of ALHYDROGEL® 85 aluminum hydroxide formulation using a Flow Cam (Particle Characterization Lab, Novato, Calif.) showed that the majority of the particles were less than 1 micron (μm) in size, with a mean diameter of 0.85 μm and a particle size distribution ranging from 0.5 μm to 24 μm. The Flow Cam analysis used a 50 μm capillary and 20× objective and the system was calibrated with a 20 μm latex bead (Thermo).

Dynamic light scattering analysis (Malvern Zetasizer Nano-S, Malvern Instruments) was performed on a series of vaccine constructs where D61-04 polynucleotide and NY-ESO-1₇₉₋₁₀₈ and NY-ESO-1₁₄₂₋₁₇₃ peptide antigens were co-adsorbed to ALHYDROGEL® 85 2% aluminum hydroxide particles in a series of mass ratios (see Experiment S5-6), as well as on ALHYDROGEL® 85 2% aluminum hydroxide particles alone. A slurry of each sample was formed by dilution of the vaccines or aluminum hydroxide particles in 10 mM NaOAc, 150 mM NaCl, pH 7.0. Table S3-0 shows that the particle diameter at the geometric mean of the population distribution curve was 1.3 μm for the aluminum hydroxide particles and in the range of 1.7 to 2.0 μm for the vaccines, consistent with the expected larger mass after co-adsorption of the polynucleotide and antigen. The data is an average derived from five independent runs with 10 measurements each. The polydispersity index of the unmodified aluminum hydroxide particles was 0.2, consistent with a moderate level of polydispersity. The polydispersity index of the vaccine particles was in the 0.39 to 0.46 range indicating more disperse particle sizes. The dynamic light scattering instrument was calibrated with a 3 μm polystyrene divinylbenzene bead (Thermo Fisher Scientific).

Although ALHYDROGEL® 85 was employed in exemplary methods, the present disclosure is in no way limited to the use of this brand of aluminum hydroxide adjuvant. Other brands and non-branded aluminum hydroxide adjuvants are also suitable for use in the methods and compositions described herein, and hence this formulation is generally referred to herein as “alum”, “aluminum hydroxide suspension”, or “aluminum hydroxide particles”.

TABLE S3-0
Dynamic Light Scattering Characterization of Vaccine Formulations and
Aluminum Hydroxide Particles in 10 mM NaOAc, 150 mM NaCl,
pH 7.0 Buffer

| Lot #/Sample | Effective diameter (μm)¹ | Polydispersity index² |
|---|---|---|
| CC-100316-01 | 2.0 | 0.41 |
| CC-100316-02 | 1.8 | 0.46 |
| CC-100316-03 | 2.0 | 0.41 |
| CC-100316-04 | 2.0 | 0.39 |
| CC-100316-05 | 1.8 | 0.41 |
| CC-100316-06 | 1.7 | 0.46 |
| Al(OH)₃ | 1.3 | 0.20 |

¹Particle diameter calculated at the geometric mean of the population distribution curve.
²Polydispersity index defined as the width/mean of the population distribution curve at the half-height point.

Prior to mixing with CpG-ODN, the aluminum hydroxide suspension was equilibrated with 10 mM sodium acetate (NaOAc), 150 mM sodium chloride (NaCl), pH 7.0, (equilibration buffer) by end-over-end mixing (˜100-150 rpm) at room temperature (20-25° C.). The minimum ratio of aluminum hydroxide suspension (i.e., aluminum content of 10±1 mg/ml) to buffer during equilibration was approximately 1 ml of aluminum hydroxide suspension to 10 ml of equilibration buffer and took place over 10-15 minutes or longer (up to 1-24 hrs). The equilibrated aluminum hydroxide was pelleted by centrifugation (3200 rpm for 15 min at 20-25° C. using a bench top centrifuge (Beckman GS-6R) fitted with a swinging bucket rotor) and the solution decanted, leaving the aluminum hydroxide complex as a wet gel pellet for storage or use in binding experiments. In most binding experiments, the amount of aluminum hydroxide used was 0.1 ml, 0.5 ml 1.0 ml, equivalent to 1 mg, 5 mg or 10 mg of aluminum, with various amounts of added CpG-ODN.

CpG-ODNs in solid form were dissolved at a nominal concentration of about 2-5 mg/mL (w/v) in 10 mM sodium acetate (NaOAc), 150 mM sodium chloride (NaCl) buffer at pH 7.0, the preferred buffer for binding to aluminum hydroxide in these examples. Since the solid CpG-ODNs contain a significant amount of associated water, the CpG-ODN concentration (mg/mL) of the solution was determined by UV spectroscopy using Beer's law and the extinction coefficients at 260 nm for each CpG-ODN: for D61-01, 24.84 mg/ml⁻¹cm⁻¹; for D61-02, 22.65 mg/ml⁻¹ cm⁻¹ and for D61-04, 30. 02 mg/ml⁻¹ cm⁻¹. A defined amount of CpG-ODN was added to a defined amount of pre-equilibrated aluminum hydroxide (Table S3-1 and Table S3-2), then additional buffer was typically added to achieve a final concentration of about 1 mg/mL based on aluminum content (although concentrations of about 0.75 to 2 mg/mL were also shown to be acceptable) and the resulting mixture was mixed end-over-end at ˜100-150 rpm for ˜2 hrs at 20-25° C. After mixing, samples were centrifuged as described above to pellet the aluminum hydroxide. The washing/centrifugation process was repeated two more times with 10 mM NaOAc, 150 mM NaCl, pH 7.0 buffer and supernatants were analyzed by UV spectrophotometry to assess CpG-ODN binding (see Example S4). CpG adsorbed to aluminum hydroxide was stored at 2-8° C. as moist pellets after the final wash.

### Experiment S3-1: D61-01 Binding to Aluminum Hydroxide in 10 mM NaOAc, 150 mM NaCl, pH 7.0 Buffer

D61-01 was adsorbed to aluminum hydroxide as described above across a range of loadings (0.2 mg to 0.9 mg of D61-01 input per 1.0 mg of aluminum hydroxide based on aluminum content) at three different scales (10 mg, 20 mg and 100 mg of aluminum) in 10 mM NaOAc, 150 mM NaCl, pH 7.0 buffer (Table S3-1). These data show successful non-covalent adsorption of D61-01 to aluminum hydroxide at high efficiency (>71-100%) for all conditions, even in the presence of 150 mM NaCl, roughly equivalent to physiological salt concentration.

TABLE S3-1
Adsorption of D61-01 to Aluminum Hydroxide in 10 mM NaOAc, 150 mM NaCl,
pH 7.0 Buffer

| | Reaction | | D61-01 | D61-01 | D61-01 | Found Al(OH)₃:D61- |
|---|---|---|---|---|---|---|
| | volume | Al(OH)₃ | input | bound | Binding | 01 ratios |
| Lot No. | (ml) | (mg)¹ | (mg) | (mg) | efficiency² | (w/w)¹ |
| 10072015 | 5 | 10 | 3.0 | 3.0 | 100% | 1:0.3 |
| 10092015 | 50 | 100 | 23 | 23 | 100% | 1:0.2 |
| 10152015 | 20 | 20 | 23 | 18.0 | 78% | 1:0.9 |
| 10162015 | 20 | 20 | 21 | 15.0 | 71% | 1:0.8 |
| 02172016 | 13 | 10 | 10 | 9.0 | 90% | 1:0.9 |

¹Weight shown reflects the weight of aluminum in Al(OH)₃.
²% Binding Efficiency = {D61-01 bound/D61-01 input} × 100 by UV analysis (see Example S4).

### Experiment S3-2: Comparison of Binding Capacities and Efficiencies of D61-01 and D61-04 to Aluminum Hydroxide in 10 mM NaOAc, 150 mM NaCl, pH 7.0 Buffer

The binding capacities and efficiencies of two different CpG-ODNs (D61-01 and D61-04) onto aluminum hydroxide were compared by independently mixing increasing amounts of each CpG-ODN (0.25, 0.5, 1.0 and 1.5 mg) with a fixed amount of aluminum hydroxide (1.0 mg based on aluminum content) in 10 mM NaOAc, 150 mM NaCl buffer at pH 7.0 as described above. For the 0.25 mg, 0.5 mg and 1 mg input amounts for both D61-01 and D61-04, the binding reaction volume was 1.0 ml. For the 1.5 mg input condition for D61-01, the binding reaction volume was 1.1 ml. For the 1.5 mg input condition for D61-04, the binding reaction volume was 1.2 ml. The two CpG-ODNs have significantly different primary and secondary structures and were therefore thought to have different binding capacities to aluminum hydroxide. D61-01 is a chimeric compound (CC) containing nucleic acid heptamers separated by HEG (hexaethylene glycol) spacers, and is single-stranded in solution. In contrast, D61-04 is a polynucleotide that contains a long palindromic sequence, and is predominantly double-stranded in solution. Under these conditions 1 mg of aluminum hydroxide was shown to bind a maximum of 1.0 mg and 1.1 mg of D61-01 and D61-04, respectively (Table S3-2). These data show that the general method for binding CpG-ODN to aluminum hydroxide is applicable to multiple types of CpG-ODN sequences (polynucleotides or chimeric compounds) with different secondary structures (single-stranded or double-stranded). The binding capacity for both types of CpG-ODNs is similar at about 1 mg of CpG-ODN per 1 mg of aluminum hydroxide, and the binding efficiency is close to 100% up until the binding capacity is reached.

TABLE S3-2
Binding Capacities and Efficiencies of D61-01 and D61-04 to 1 mg of
Aluminum Hydroxide in 10 mM NaOAc, 150 mM NaCl,
pH 7.0 Buffer¹

| D61-01 | | | D61-04 | | |
|---|---|---|---|---|---|
| Input | Bound | Binding | Input | Bound | Binding |
| (mg) | (mg) | efficiency² | (mg) | (mg) | efficiency² |
| 0.25 | 0.25 | 100% | 0.25 | 0.25 | 100% |
| 0.50 | 0.50 | 100% | 0.50 | 0.50 | 100% |
| 1.0 | 0.99 | 99% | 1.0 | 0.89 | 89% |
| 1.5 | 0.97 | 65% | 1.5 | 1.1 | 73% |

¹Weight of aluminum hydroxide is based on aluminum content.
²Binding Efficiency (%) = {CpG-ODN bound/CpG-ODN input} × 100 by UV analysis method (see Example S4).

### Example S4: Procedure to Quantify CpG-ODN Adsorbed to Aluminum Hydroxide and the CpG-ODN:Aluminum Hydroxide Ratio (w/w)

The ratio of CpG-ODN adsorbed to aluminum hydroxide (% binding efficiency) was quantified by UV absorbance at 260 nm (A₂₆₀) using Equation 1 and Equation 2. Samples were diluted to be in the linear range of the UV detector in 10 mM NaOAc, 150 mM NaCl buffer, pH 7 and the spectrophotometer was blanked against the same buffer. The A₂₆₀ input and A₂₆₀ supernatant were determined by multiplication of the found A₂₆₀ value by the dilution factor and the pre-dilution volume, which was then used in Equation 1. The weight/weight (w/w) CpG-ODN to aluminum hydroxide [Al(OH)₃] ratio was calculated using Equation 2.

$$\begin{matrix}\% CpG \text{-} ODN Adsorbed ( \% Binding efficiency ) = \frac{( {A}_{260} input - {A}_{260} supernatant ) \times 100}{{A}_{260} input} & Equation 1 \\ CpG \text{-} ODN \text{:} {Al ( OH )}_{3} Ratio ( w \text{/} w ) = \frac{Weight CpG \text{-} ODN input \times \% CpG \text{-} ODN Adsorbed}{Weight {Al ( OH )}_{3} input \times 100} & Equation 2\end{matrix}$$

### Experiment S4-1: Effect of Buffer pH on Binding of Varying Mass of D61-04 to Aluminum Hydroxide Particles

The effect of buffer pH from 6.5 to 7.5 on the binding of varying mass of D61-04 polynucleotide to aluminum hydroxide particles was assessed. Three stock solutions were made with D61-04 polynucleotide at 5 mg/mL in 10 mM NaOAc, 150 mM NaCl buffer that was pH 6.5, 7.0 or 7.5. The exact concentration was determined by the UV absorbance method described in Example S4. These stock solutions were then further diluted with the corresponding buffer as needed to obtain a range of D61-04 polynucleotide working solutions of the appropriate concentration. Then, a series of 1:1 (v/v) slurries containing 1.0, 1.5 or 2.0 mg of D61-04 polynucleotide in 10 mM NaOAc, 150 mM NaCl, pH 6.5, 7.0 or 7.5 and a solution containing 1.0 mg aluminum hydroxide particles in 10 mM NaOAc, 150 mM NaCl, pH 6.5, 7.0 or 7.5 were mixed in an end-over-end mixer at ˜15 rpm for ˜3 hrs at 20-25° C. Unbound D61-04 polynucleotide was separated from the aluminum hydroxide particles by centrifugation as described in Experiment S5-3. The washing/centrifugation process was repeated two more times with the same buffer used for binding, and the pooled supernatants were analyzed for unbound D61-04 polynucleotide by the UV absorbance method. The percent of input D61-04 polynucleotide bound to the Al(OH)₃ particle was maximal at the 1:1 ratio of D61-04 polynucleotide incubated with Al(OH)₃. This percent bound was reduced ˜15% at the 1.5:1 ratio and further reduced 10-15% at the 2:1 ratio. These effects were independent of pH over the range tested (Table S4-1).

TABLE S4-1
Adsorption of Varying Mass of D61-04 to Aluminum Hydroxide
Particles in 10 mM NaOAc, 150 mM NaCl, pH 6.5, 7.0 or 7.5 Buffer

| | D61-04 | Al(OH)₃ | % D61-04 |
|---|---|---|---|
| pH | Input (mg)¹ | Input (mg)² | Bound³ |
| 6.5 | 1.0 | 1.0 | 92 |
| 7.0 | 1.0 | 1.0 | 91 |
| 7.5 | 1.0 | 1.0 | 92 |
| 6.5 | 1.5 | 1.0 | 78 |
| 7.0 | 1.5 | 1.0 | 73 |
| 7.5 | 1.5 | 1.0 | 74 |
| 6.5 | 2.0 | 1.0 | 63 |
| 7.0 | 2.0 | 1.0 | 61 |
| 7.5 | 2.0 | 1.0 | 60 |

¹Mass of input D61-04 polynucleotide by UV analysis method
²Mass of Al(OH)₃ is based on input aluminum content
³Percent (%) bound = {polynucleotide bound/polynucleotide input} × 100, by UV analysis method.

### Example S5: Procedure for Adsorption of Peptide Antigens to Aluminum Hydroxide

Unlike CpG-ODNs, many peptide antigens of interest are highly hydrophobic and are insoluble at useful concentrations in aqueous buffers commonly employed for binding to Alum. Two peptides with different levels of hydrophobicity were used in initial studies of binding to aluminum hydroxide: OVApep with ˜38% hydrophobicity and Triple melanoma (Triple) with ˜60% hydrophobicity (Table S2-1). OVApep was found to be soluble at ˜2-4 mg/ml in 0.1 M sodium bicarbonate, pH 8 buffer, a suitable buffer for adsorption to aluminum hydroxide, although it was not appreciably soluble in water, phosphate buffered saline (PBS) or sodium acetate (NaOAc) buffers. In contrast, the Triple peptide was not appreciably soluble in 0.1 M sodium bicarbonate, pH 8 buffer, water, PBS, sodium bicarbonate or NaOAc buffers. The Triple peptide was soluble at ˜1-5 mg/ml in either 6M guanidine hydrochloride (GuHCl)/pH 8.5 or 20% isopropyl alcohol (IPA)/water (v/v). However, 6M GuHCl is not a suitable solution for electrostatic adsorption of peptides to aluminum hydroxide due to its high ionic strength, and the effect of IPA on peptide binding to aluminum hydroxide was unknown.

Aluminum hydroxide was equilibrated in 10 mM NaOAc, 150 mM NaCl, pH 7.0 buffer as described in Example S3. Solid peptides were dissolved at a nominal concentration of about 2-4 mg/mL (w/v) in the indicated solution. A defined amount of peptide was added to a defined amount of pre-equilibrated aluminum hydroxide (Table S5-1 and Table S5-2), then typically more of the same solution was added to achieve a final concentration of about 1 mg/mL based on aluminum content and the resulting mixture was mixed end-over-end at ˜100-150 rpm for ˜2-18 hrs at 20-25° C. After mixing, samples were centrifuged as described above to pellet the aluminum hydroxide. The washing/centrifugation process was repeated two more times with the same solution used for binding, and supernatants were analyzed by UV spectrophotometry and/or amino acid analysis to assess peptide binding (see Example S6). Peptide adsorbed to aluminum hydroxide was stored at 2-8° C. as moist gel pellets after the final wash.

### Experiment S5-1: Binding OVApep to Aluminum Hydroxide in 0.1 M Sodium Bicarbonate, pH 8.0 Buffer

OVApep was adsorbed to aluminum hydroxide (equilibrated in 0.1 M sodium bicarbonate, pH 8.0) at two loadings (0.98 mg and 0.44 mg input per 1.0 mg of aluminum hydroxide based on aluminum content) at two different scales (10 mg and 50 mg of aluminum in aluminum hydroxide) in 0.1 M sodium bicarbonate, pH 8.0 buffer (Table S5-1). These data show successful non-covalent adsorption of OVApep to aluminum hydroxide. Under these conditions, the maximum binding capacity was 0.3 mg of OVApep per 1.0 mg of aluminum hydroxide based on aluminum content.

TABLE S5-1
Adsorption of OVApep to Aluminum Hydroxide in 0.1M Sodium Bicarbonate,
pH 8.0 Buffer

| | Reaction | | OVApep | OVApep | OVApep | Found Al(OH)₃:OVApep |
|---|---|---|---|---|---|---|
| | volume | Al(OH)₃ | input | bound | binding | ratios |
| Lot No. | (ml) | (mg)¹ | (mg) | (mg) | efficiency | (w/w)¹ |
| 09212015 | 10 | 10 | 9.8 | 3.3 | 34% | 1:0.3 |
| 10122015 | 50 | 50 | 22.0 | 15.0 | 68% | 1:0.3 |

¹Weight of Al(OH)₃ is based on aluminum content
²Binding Efficiency (%) = {Peptide bound/Peptide input} × 100 by UV (see Example S6)

### Experiment S5-2: Binding Triple Melanoma (Triple) Peptide to Aluminum Hydroxide in Isopropyl Alcohol/Water

The Triple peptide was adsorbed to aluminum hydroxide (equilibrated in NaOAc buffer) at a loading of 0.80 mg input per 1.0 mg of aluminum hydroxide based on aluminum content at a 10 mg scale (Table S5-2). The Triple peptide dissolved in 20% IPA/water (v/v) was mixed 1:1 (v/v) with aluminum hydroxide in 10 mM NaOAc, 150 mM NaCl, pH 7.0 buffer, providing a final composition of 10% IPA in 5 mM NaOAc, 75 mM NaCl, pH 7.0 buffer (v/v). These data show successful non-covalent adsorption of the Triple peptide to aluminum hydroxide. Under these conditions the peptide adsorbed to aluminum hydroxide with 100% binding efficiency.

TABLE S5-2
Adsorption of Triple Melanoma (Triple) Peptide to Aluminum Hydroxide in 10%
IPA in 5 mM NaOAc, 75 mM NaCl, pH 7.0 Buffer (v/v)

| | Reaction | | Triple | Triple | | |
|---|---|---|---|---|---|---|
| | volume | Al(OH)₃ | Input | bound | Triple binding | Found Al(OH)₃:Triple |
| Lot No. | (ml) | (mg)¹ | (mg) | (mg) | efficiency | Ratio (w/w)¹ |
| 11092015 | 19 | 10 | 8.0 | 8.0 | 100% | 1:0.8 |

¹Weight of Al(OH)₃ is based on aluminum content
²Binding Efficiency (%) = {Peptide bound/Peptide input} × 100 by UV (see Example S6)

### Experiment S5-3: Binding of a Constant Mass of NY-ESO-1 OLP Antigens to a Varying Mass of Aluminum Hydroxide Particles

The effect of varying the mass of aluminum hydroxide on the adsorption of a fixed quantity of the NY-ESO-1₇₉₋₁₀₈ and NY-ESO-1₁₄₂₋₁₇₃ OLP antigens was assessed to determine the minimum mass of aluminum hydroxide particle required to efficiently co-adsorb a specific quantity of the OLP antigens. NY-ESO-1₇₉₋₁₀₈ and NY-ESO-1₁₄₂₋₁₇₃ OLP antigens were dissolved in 20% isopropanol (IPA)/water (v/v) at ˜1 mg/ml and the exact concentration was determined by amino acids analysis. This stock solution was then further diluted with 20% (v/v) IPA/water as needed to obtain a range of working solutions of the appropriate concentration. Then, a 1:1 (v/v) solution containing 0.2 mgs total OLP antigens (0.1 mg of each OLP antigen) and 1.0, 0.8, 0.6, 0.4 or 0.2 mgs aluminum hydroxide particles in 10 mM NaOAc, 150 mM NaCl, pH 7.0 was mixed to yield a final buffer composition of 10% IPA (v/v) in 5 mM NaOAc, 75 mM NaCl, pH 7.0 buffer. The resulting slurry was mixed end-over-end at ˜15 rpm for ˜3 hours at 20-25° C., and the aluminum hydroxide-bound OLP antigens were separated from unbound peptides by centrifugation at ˜3200 rpm for 15 min at 20-25° C. using a Beckman GS-6R centrifuge fitted with a swinging bucket rotor. The washing/centrifugation process was repeated two more times with the same buffer used for binding, and the pooled supernatants were analyzed for OLP antigen content by amino acid analysis to assess overall OLP antigen binding (see Example S6). Under these conditions, the 0.2 mgs of NY-ESO-1₇₉₋₁₀₈ and NY-ESO-1₁₄₂-173 OLP antigens bound at comparable levels of >85% binding under conditions with 1.0 mg, 0.8 or 0.6 mgs of the aluminum hydroxide particles (Table S5-3). However, binding of the NY-ESO-1 antigens diminished to 73% for the conditions with 0.4 and 0.2 mg of aluminum hydroxide particles.

TABLE S5-3
Adsorption of NY-ESO-1 OLP Antigens to Aluminum Hydroxide
Particles in 10% (v/v) IPA in 5 mM NaOAc, 75 mM NaCl, pH 7.0 Buffer

| | | NY-ESO- | | % OLP |
|---|---|---|---|---|
| | Al(OH)₃ | 1₇₉₋₁₀₈ | NY-ESO-1₁₄₂₋₁₇₃ | antigen |
| Lot No. | Input (mg)¹ | Input (mg)² | Input (mg)² | bound³ |
| CC-100316-01 | 1.0 | 0.1 | 0.1 | 89 |
| CC-100316-02 | 0.8 | 0.1 | 0.1 | 89 |
| CC-100316-03 | 0.6 | 0.1 | 0.1 | 87 |
| CC-100316-04 | 0.4 | 0.1 | 0.1 | 73 |
| CC-100316-05 | 0.2 | 0.1 | 0.1 | 73 |

¹Mass of Al(OH)₃ is based on input aluminum content
²Mass of NY-ESO-1 OLP antigens is by amino acid analysis
³Percent (%) antigen bound = {Peptide bound/Peptide input} × 100, by amino acid analysis method (see Example S6)

### Experiment S5-4: Binding of Increasing Mass of NY-ESO-1 OLP Antigens to Two Masses of Aluminum Hydroxide Particles

The effect of increasing mass of NY-ESO-1₇₉₋₁₀₈ and NY-ESO-1₁₄₂₋₁₇₃ OLP antigens on the adsorption to aluminum hydroxide particles was assessed to determine the maximum mass of antigen that could be co-adsorbed. The NY-ESO-1₇₉₋₁₀₈ and NY-ESO-1₁₄₂₋₁₇₃ OLP antigens were dissolved in 20% IPA/water (v/v) at ˜1 mg/ml and the exact concentration was determined by amino acids analysis. This stock solution was then further diluted with 20% (v/v) IPA/water as needed to obtain a range of working solutions of the appropriate concentration. Then, a 1:1 (v/v) solution containing 0.4, 0.5 and 0.6 mgs total OLP antigens (0.2, 0.25 and 0.3 mgs of each OLP) and 0.5 and 1.0 mgs aluminum hydroxide particles in 10 mM NaOAc, 150 mM NaCl, pH 7.0 was mixed to provide a final buffer composition of 10% IPA in 5 mM NaOAc, 75 mM NaCl, pH 7.0 buffer (v/v). The resulting slurry was mixed end-over-end at ˜15 rpm for ˜3 hrs at 20-25° C., and then the aluminum hydroxide-bound OLP antigens were separated from unbound peptide by centrifugation as described in Experiment S5-3. The washing/centrifugation process was repeated two more times with the same buffer used for binding, and the pooled supernatants were analyzed for OLP antigen content by amino acid analysis to assess overall OLP antigen binding (see Example S6). Under these conditions, the increasing masses of pooled NY-ESO-1₇₉₋₁₀₈ and NY-ESO-1₁₄₂₋₁₇₃ OLPs bound with comparable and ˜85% binding efficiency to 0.5 or 1.0 mg aluminum hydroxide particles (Table S5-4).

TABLE S5-4
Adsorption of NY-ESO-1 OLP Antigens to Aluminum Hydroxide
Particles in 10% (v/v) IPA in 5 mM NaOAc, 75 mM NaCl, pH 7.0 Buffer

| | | NY-ESO- | | % OLP |
|---|---|---|---|---|
| | Al(OH)₃ | 1₇₉₋₁₀₈ | NY-ESO-1₁₄₂₋₁₇₃ | antigen |
| Lot No. | Input (mg)¹ | Input (mg)² | Input (mg)² | Bound³ |
| CC-261016-05 | 1.0 | 0.2 | 0.2 | 85 |
| CC-261016-03 | 1.0 | 0.25 | 0.25 | 84 |
| CC-261016-01 | 1.0 | 0.3 | 0.3 | 87 |
| CC-261016-06 | 0.5 | 0.2 | 0.2 | 85 |
| CC-261016-04 | 0.5 | 0.25 | 0.25 | 84 |
| CC-261016-02 | 0.5 | 0.3 | 0.3 | 84 |

¹Mass of Al(OH)₃ is based on input aluminum content
²Mass of NY-ESO-1 OLP antigens by amino acid analysis
³Percent (%) OLP antigen bound = {Peptide bound/Peptide input} × 100, by amino acid analysis method

### Experiment S5-5 (Part 1). Binding of Equimolar Amounts of Three Different NY-ESO-1 Peptide Antigens to Aluminum Hydroxide Particles

The effect of combining equimolar amounts of three different NY-ESO-1 peptide antigens, NY-ESO-1₇₉₋₁₀₈, NY-ESO-1₁₂₁₋₁₅₀ and NY-ESO-1₁₄₂₋₁₇₃ (SEQ ID NOs. 55, 57 and 58 respectively), on their adsorption to 0.5 mg of aluminum hydroxide particles (mass expressed as elemental aluminum) was assessed to determine the efficiency of co-adsorption of each of the three peptides. Individual peptide antigens were dissolved in at ˜1 mg/mL of 20% isopropanol (IPA)/80% water (v/v) and the exact concentration of the mixture was then determined by amino acids analysis. For this example, a defined amount of each peptide solution was mixed to achieve 1.34 μmoles of each peptide relative to the NY-ESO-1₁₄₂₋₁₇₃ peptide antigen. This mixture of the three peptides was sampled and the total antigen concentration determined by amino acids analysis. Approximately 12.6 mg (input) of total peptide antigens (˜4.0 mg of each NY-ESO-1 antigen) in 20% IPA/80% water (v/v) was mixed 1:1 with 10 mg of aluminum hydroxide in 10 mM NaOAc, 150 mM NaCl, pH 7.0 buffer to yield a final buffer composition of 10% IPA (v/v) in 5 mM NaOAc, 75 mM NaCl, pH 7.0 buffer. This solution was mixed using a rotary spinner at 15 rpms for about 3 hours at room temperature to allow adsorption of the peptides to the aluminum hydroxide particles. After 3 hours the aluminum hydroxide-bound antigens were separated from non-adsorbed antigens by centrifugation at ˜3200 rpm for 15 min at 20-25° C. using a Beckman GS-6R centrifuge fitted with a swinging bucket rotor. The supernatant, containing the unbound antigen fraction, was decanted and subjected to amino acid analysis. The wet gel pellet of aluminum hydroxide-bound antigens was reconstituted in 20 ml of 10 mM NaOAc, 150 mM NaCl, pH 7.0 buffer, mixed briefly (1-5 min), centrifuged as before, and the supernatant decanted (wash) and subjected to amino acid analysis. Finally the washed aluminum hydroxide with bound antigens was also subjected to amino acid analysis. Under these conditions, the mixture of NY-ESO-1₇₉₋₁₀₈, NY-ESO-1₁₂₁₋₁₅₀ and NY-ESO-1₁₄₂₋₁₇₃ peptide antigens bound to aluminum hydroxide particles with an efficiency of 95-97%, determined both indirectly (by subtracting the input quantity of antigen from the quantity of antigen observed in the unbound and wash fractions by amino acid analysis) and directly (by amino acid analysis of the antigen bound to the aluminum hydroxide particles) (Table S5-5a).

TABLE S5-5a
Co-adsorption of Three NY-ESO-1 Peptide Antigens to Aluminum
Hydroxide Particles in 10% (v/v) IPA in 5 mM NaOAc, 75 mM NaCl,
pH 7.0 Buffer

| | | NY-ESO- | NY-ESO- | NY-ESO- | |
|---|---|---|---|---|---|
| | Al(OH)₃ | 1₇₉₋₁₀₈ | 1₁₂₁₋₁₅₀ | 1₁₄₂₋₁₇₃ | |
| | Input | Input | Input | Input | % total antigen |
| Lot No. | (mg)¹ | (mg)² | (mg)² | (mg)² | bound³ |
| BM- | 10 | 4.1 | 4.1 | 4.6 | Indirect = 95% |
| 012517 | | | | | Direct = 97% |

¹Mass of Al(OH)₃ input is based on aluminum content.
²Mass of combined NY-ESO-1 peptide antigens is by amino acid analysis. The total antigen input was 12.6 mg
³Percent (%) antigen bound (indirect method) = {Peptide bound/Peptide input} × 100 by amino acid analysis method. Percent (%) antigen bound (direct method) after hydrolyzing the aluminum-bound peptides, followed by centrifugation to separate aluminum particles then performing amino acid analysis on the supernatant.

### Experiment S5-5 (Part 2). Co-Adsorption of Different Amounts of CpG (D61-04) to a Fixed Amount of Aluminum Hydroxide-bound NY-ESO-1 Peptide Antigens

The effect of adding three different masses of D61-04 (SEQ ID NO: 6) to a fixed mass of aluminum hydroxide particle with bound NY-ESO-1 peptide antigens (described in S5-5, Part 1) was assessed to determine the binding capacity of D-61-04 to the aluminum hydroxide particle with bound peptide antigens. D61-04 solutions at 4, 2 and 1 mg/ml were prepared from a concentrated stock solution by dilution with 10 mM NaOAc, 150 mM NaCl, pH 7 buffer. Next, 0.5 ml of each D61-04 solution was mixed 1:1 (v/v) with a slurry of aluminum hydroxide with bound peptide antigens containing ˜1.2 mgs total peptide antigens (˜0.4 mg of each antigen) bound per mg of aluminum hydroxide. These three experimental solutions (˜1.0 ml each) were then mixed using a rotary spinner at 15 rpms for ˜3 hours at room temperature to facilitate co-adsorption of the D61-04 to the aluminum hydroxide particle with bound NY-ESO-1 antigens. Unbound D61-04 was separated by centrifugation at ˜3200 rpm for 15 min at 20-25° C. using a Beckman GS-6R centrifuge fitted with a swinging bucket rotor. The D61-04 unbound fraction was analyzed by UV spectroscopy and the amount of D61-04 bound to aluminum hydroxide was deduced by subtracting the unbound from input amounts. Next, the D61-04/peptide antigen-bound aluminum hydroxide particles were reconstituted in 10 mLs of 10 mM NaOAc, 150 mM NaCl, pH 7 buffer, mixed for 1-5 min, and centrifuged as above to separate unbound D61-04 and antigens (believed to be displaced by D61-04 due to a competing phosphate ligand exchange mechanism). The unbound and wash fractions were each analyzed by UV spectroscopy to determine the total amount of unbound D61-04. The amount of D61-04 co-adsorbed to aluminum hydroxide-bound antigens was deduced as the difference between the D61-04 input and unbound D61-04 (unbound+wash fractions). Under these conditions, the binding capacity of D61-04 was 1.8, 1.3 and 1.0 mg of D61-04 per mg of aluminum hydroxide particles with bound antigens, respectively for the three different co-adsorption mixtures (Table S5-5b). The final amounts (mg) and binding ratios for the various components in these related particles suggest that the antigens were not displaced by excess D61-04 (i.e., the amount of bound antigens held constant at approximately 0.6 mg of total antigens per 0.5 mg of aluminum hydroxide particle).

TABLE S5-5b
Summary of Co-adsorption of D61-04 to Aluminum Hydroxide-Bound
Peptide Antigens in 10 mM NaOAc, 150 mM NaCl, pH 7.0 Buffer

| | | Binding Ratios | | |
|---|---|---|---|---|
| | | D61-04:Aluminum Hydroxide:Antigens | | |
| | | (mg) | | |
| | D61-04 input | D61-04 bound | Al(OH)₃ | Antigens* |
| Lot No. | (mg/ml) | (mg) | (mg) | (mg) |
| BM-012517-1 | 4 | 1.8 | 0.5 | 0.6 |
| BM-012517-2 | 2 | 1.3 | 0.5 | 0.55 |
| BM-012517-3 | 1 | 1.0 | 0.5 | 0.6 |

*Total amount (mg) of three adsorbed peptide antigens (NY-ESO-1₇₉₋₁₀₈, NY-ESO-1₁₂₁₋₁₅₀ and NY-ESO-1₁₄₂₋₁₇₃) determined by triplicate amino acid analysis measurements.

### Experiment S5-6. Binding of Equimolar Amounts of Two NY-ESO-1 Peptide Antigens Plus MAGE A10 Peptide Antigen and D61-04 CpG (D64-01) to Aluminum Hydroxide Particles

In an experiment similar to S5-5 part 1, the NY-ESO-1₁₂₁₋₁₅₀ peptide antigen was substituted with the MAGE A10₂₄₅₋₂₇₄ peptide antigen. Otherwise all other experimental conditions for co-adsorbing this set of three antigens to aluminum hydroxide particles were the same. In part 1 of this experiment the combined NY-ESO-1₇₉₋₁₀₈, NY-ESO-1₁₄₂₋₁₇₃ and MAGE A10₂₄₅₋₂₇₄ peptide antigens (SEQ ID NOs:55, 58 and 50), representing ˜14 mg total antigen input, bound to aluminum hydroxide particles with an overall binding efficiency of 93% as determined by amino acid analysis (Table S5-6a).

TABLE S5-6a
Co-adsorption of Two NY-ESO-1 Peptide Antigens plus MAGE A10 to
Aluminum Hydroxide Particles in 10% (v/v) IPA in 5 mM NaOAc, 75 mM NaCl, pH 7.0 Buffer

| | Al(OH)₃ | | | | % total |
|---|---|---|---|---|---|
| | Input | NY-ESO-1_{79 - 108} | MAGE A10_{245 - 274} | NY-ESO-1₁₄₂₋₁₇₃ | Antigen |
| Lot No. | (mg)¹ | Input (mg)² | Input (mg)² | Input (mg)² | Bound³ |
| BM- | 10 | 4.5 | 4.5 | 5.0 | Indirect = 91% |
| 220117 | | | | | Direct = 93% |

¹Mass of Al(OH)₃ input is based on aluminum content.
²Mass of NY-ESO-1 and MAGE peptide antigens is by amino acid analysis. The total peptide antigen input was 12.6 mg.
³Percent (%) antigen bound (indirect method) = {Peptide bound/Peptide input} × 100 by amino acid analysis method. Percent (%) antigen bound (direct method) after hydrolyzing the aluminum-bound peptide peptides, followed by centrifugation to separate aluminum particles then performing amino acid analysis on the supernatant.

In part 2 of this experiment 4, 8 & 12 mg/mL of D61-04 CpG were added into three separate co-adsorption reactions to assess CpG binding and the potential of D61-04 (in excess of the theoretical binding capacity to aluminum hydroxide particles) to displace aluminum hydroxide particle-bound peptide antigens. The other experimental conditions were as described in S5-5, part 2. Under these conditions, 0.8, 1.1 and 1.7 mg of D61-04 were adsorbed to the aluminum hydroxide particle-bound peptide antigens (Table S5-6b). In addition, the addition of excess D61-04 resulted in little to no displacement of aluminum hydroxide particle-bound antigens as assessed by assaying the unbound and wash fractions for the presence of peptide antigens by amino acid analysis.

TABLE S5-6b
Co-adsorption of D61-04 to Al(OH)₃-Bound Peptide Antigens
(NY-ESO-1_{79 - 108}, NY-ESO-1_{142 - 173} and MAGE A10_{245 - 274}) in
10 mM NaOAc, 150 mM NaCl, pH 7.0 Buffer

| | | Binding ratios of | | |
|---|---|---|---|---|
| | D61-04 | D61-04:aluminum hydroxide:antigens (mg) | | |
| | input | D61-04 bound | | Antigens* |
| Lot No. | (mg/ml) | (mg) | Al(OH)₃ (mg) | (mg) |
| BM-220117-1 | 4 | 0.8 | 0.5 | 0.59 |
| BM-220117-2 | 8 | 1.1 | 0.5 | 0.65 |
| BM-220117-3 | 12 | 1.7 | 0.5 | 0.59 |

*Total amount (mg) of three combined NY-ESO-1 peptide antigens (NY-ESO-1_{79 - 108}, NY-ESO-1_{142 - 173} and MAGE A10_{245 - 274}) as determined by triplicate amino acid analysis measurements.

### Experiment S5-7: NY-ESO-1 Peptide Antigens Remain Co-Adsorbed to Aluminum Hydroxide Particles Following Terminal Sterilization by Autoclaving

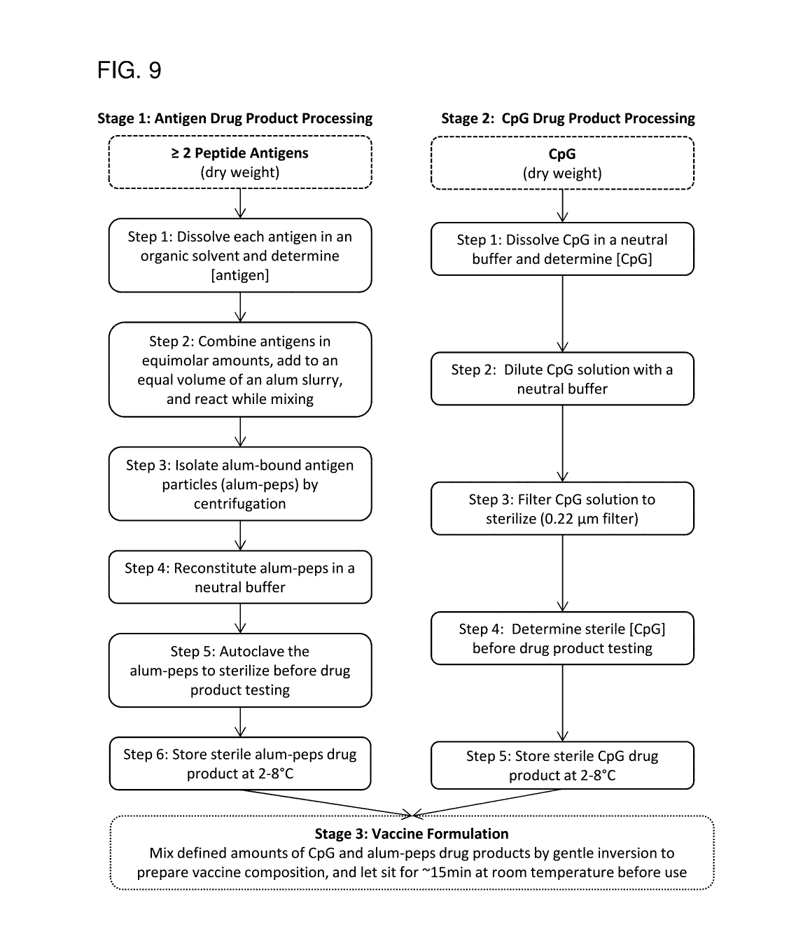

The manufacturing scheme for CpG-Alum-Peptide conjugates is provided in FIG. 9. Aluminum drug products composed of protein-based antigens co-adsorbed to aluminum hydroxide particles (0.5-3 μm diameter size range) cannot be sterilized by 0.2 micron (μm) filtration as the final processing step in manufacturing (terminal sterilization). Accordingly a more cumbersome aseptic processing approach is required. Since the aluminum hydroxide particles used in vaccines are terminally sterilized by autoclaving, whether peptide antigens of about 25-35 amino acids in length would remain co-adsorbed to the particle after terminal sterilization by autoclaving was assessed. A slurry of the three NY-ESO-1 peptide antigens co-adsorbed to aluminum hydroxide particles (lot # BM-012517), manufactured as described in Experiment S5-5 part1) was exposed to high-pressure saturated steam at 121° C. for 15-20 minutes (autoclaving). The size distribution and polydispersity index of the particles assessed by dynamic light scattering was similar before and after autoclaving. The aluminum hydroxide particles in the slurry were removed by centrifugation, before and after autoclaving, and the peptide antigen content of the supernatant was quantitated by both reversed-phase (RP) HPLC with absorbance detection @ 215 nm and direct spectrophotometric measurement @ 215 nm. Direct spectrophotometric analysis indicated that <1% of the peptide antigens had dissociated from the aluminum hydroxide particles after autoclaving (Table S5-7). RP-HPLC analysis assesses the quantity of each of the three peptide antigens in the supernatant since they have unique retention times. This analysis demonstrated that the integrated peak value for each peptide was at or below the lower limit of quantitation of the assay (>10 μg/ml), indicating that <5% of the input amount of each of the individual peptide antigens was desorbed by autoclaving. Both methods indicate that terminal sterilization of the NY-ESO-1 peptide antigens co-adsorbed to aluminum hydroxide particles using conventional autoclaving methodology neither alters the particle size/size distribution nor significantly desorbs the three NY-ESO-1 peptide antigens.

TABLE S5-7
Desorption of Three NY-ESO-1 Peptide Antigens from
Aluminum Hydroxide Particles by Autoclaving

| | Peptide | |
|---|---|---|
| | antigens in supernatant by | Peptide antigens |
| | spectrophotometric | in supernatant by |
| Condition | assay (mg/ml)* | RP-HPLC assay** |
| Before autoclaving | 0.019 | <10 μg/ml |
| After autoclaving | 0.026 | <10 μg/ml |

*Amount of absorbance for all three peptide antigens estimated by UV @ 215 nm. Absorbance of a solution of the three peptide antigens not bound to aluminum hydroxide particles gave value of 18.8 (corresponding to 0.6 mg/mL concentration, confirmed by amino acid analysis).
**The lower limit of quantitation for this assay is ~10 μg/ml per peptide (<5% of each peptide input).

### Experiment S5-8. Evaluation of Selected Organics Solvents for the Solubilization of Human Peptide Antigens

Approximately 1 mg of four different peptide antigens, NY-ESO-1₇₉₋₁₀₈, NY-ESO-1₁₂₁₋₁₅₀, NY-ESO-1₁₄₂₋₁₇₃, and MAGE A10₂₄₅₋₂₇₄, were separately weighed out and placed in borosilicate glass test tubes. Peptide antigen solubility was assessed by adding 1 ml of six different organic solvents, 20% isopropyl alcohol (IPA)/80% water (v/v), 100% dimethyl sulfoxide (DMSO), 20% DMSO/80% water (v/v), 100% dimethyformamide (DMF), 20% DMF/80% water (v/v), and 100% acetonitrile (ACN). After adding the solvent to each of the 4 antigens they were mixed for 3-5 seconds by intermittent vortexing at room temperature and then assessed for visual clarity within 1-2 minutes. The 100% and 20% DMSO, and 20% DMF, solvents readily solubilized all four antigens yielding a visually clear solution (Table S5-8). The 100% DMF and 20% IPA solvent yielded slightly hazy solutions or solutions with some visible particle or flake material. The 100% acetonitrile solvent failed to fully solubilize any of the four human tumor associated antigens, leaving visually observable flakes of undissolved material comparable in mass to before the solvent was added. These solubility observations were unchanged after sitting for an additional 1 or 24 hours at room temperature.

TABLE S5-8
Solubilization of Selected Peptide Antigens with Various Organic Solvents

| | 20% | 100% | 20% | 100% | 20% | 100% |
|---|---|---|---|---|---|---|
| Peptides/Solvent | IPA | DMSO | DMSO* | DMF | DMF** | ACN |
| NY-ESO-1₇₉₋₁₀₈ | clear | clear | clear | clear | clear | mostly |
| | | | | | | insoluble |
| NY-ESO-1₁₂₁₋₁₅₀ | slightly | clear | clear | clear w/ | clear | mostly |
| | hazy | | | particles | | insoluble |
| NY-ESO-1₁₄₂₋₁₇₃ | clear | clear | clear | clear | clear | mostly |
| | | | | | | insoluble |
| MAGE A10₂₄₅₋₂₇₄ | clear with | clear | clear | clear w/ | clear | mostly |
| | flakes | | | particles | | insoluble |

*Samples in 100% DMSO were diluted 1:5 with water to achieve a 20% DMSO/water solution.
**Samples in 100% DMF were diluted 1:5 with water to achieve a 20% DMF/water solution.

### Experiment S5-9. Evaluation of Selected Class 2 & 3 Solvents to Assess Solubilization of NY-ESO-1₁₄₂₋₁₇₃ Peptide Antigen and Binding to Aluminum Hydroxide Particles

Class 3 solvents are classified by the U.S. Food and Drug Administration, and other regulatory authorities, due to their known safety profiles (International Conference on Harmonization of Technical Requirements for Registration of Pharmaceuticals for Human Use guidance for industry Q3C—Tables and List [February 2012, Revision 2]). Based on their toxicology profile Class 3 solvents have a permissible impurity limits in human drug products manufactured under GMP conditions of no more than 5000 ppm [0.5% w/w]. Ten water-miscible class 3 solvents were surveyed for their ability to efficiently solubilize the NY-ESO-1₁₄₂₋₁₇₃ peptide antigen (using a visual assessment, see Table S5-9 footnote *), and for the ability of solubilized peptide antigen to then adsorb to aluminum hydroxide particles. Formic acid, ethanol, acetone and acetic acid, each at 100%, were scored a ‘1’ since they displayed visually clear solutions almost immediately. Isopropyl alcohol and 2-butanol, each at 100%, were scored a ‘1.5’ since they were also very effective at dissolving peptide antigen, with only a few insoluble flakes observed. Moreover, when the above mentioned six solvents were diluted further to 20% solvent/80% water (v/v) the NY-ESO-1₁₄₂₋₁₇₃ peptide antigen remained visually soluble (data not shown). In contrast, methyl acetate, isopropyl acetate, n-Butyl acetate and isoamyl alcohol, each at 100%, did not solubilize this peptide antigen.

The binding of NY-ESO-1₁₄₂₋₁₇₃ peptide antigen to aluminum hydroxide particles, employing the process conditions described in Experiment S5-5 (part1) except only using the single peptide antigen, was assessed for the peptide solubilized in 20% solvent/80% water (v/v); where the solvent was either formic acid, isopropyl alcohol, ethanol, acetone and acetic acid. Binding efficiency was determined by RP-HPLC measurement of the unbound peptide antigen in the supernatant, and calculated as described in Table S5-9. The NY-ESO-1₁₄₂₋₁₇₃ peptide antigen demonstrated 100% binding efficiency to aluminum hydroxide particles in all five solvents.

TABLE S5-9
Evaluation of Various Class 3 Solvents for their Capacity to Solubilize
NY-ESO-1₁₄₂₋₁₇₃ peptide Antigen and
Promote Adsorption to Aluminum Hydroxide

| | | | (%) binding |
|---|---|---|---|
| | | | efficiency to |
| | Qualitative | | aluminum |
| Class 3 Solvents | Solubility score* | Visual Appearance | hydroxide** |
| Methyl acetate | 3 | hazy/insoluble | N/D |
| Isopropyl acetate | 3 | hazy/insoluble | N/D |
| n-Butyl acetate | 3 | hazy/insoluble | N/D |
| Formic acid | 1 | clear | 100% |
| Isopropyl alcohol | 1.5 | clear with a few | 100% |
| | | insoluble flakes | |
| Ethanol | 1 | clear | 100% |
| Isoamyl alcohol | 3 | hazy | N/D |
| 2-Butanol | 1.5 | clear with a few | 100% |
| | | insoluble flakes | |
| Acetone | 1 | clear | 100% |
| Acetic acid | 1 | clear | 100% |

*1 = fully soluble/clarity similar to water, 2 = partially soluble, and 3 = insoluble.
**percent bound = % bound/input × 100 based on RP-HPLC assay at 215 nm; ND = not determined due to lack of solubility at ~1 mg/ml

### Example S6: Procedure to Quantify Peptide Adsorbed to Aluminum Hydroxide and the Peptide:Aluminum Hydroxide Ratio (w/w)

UV procedure. The amount of peptide adsorbed to aluminum hydroxide (% binding efficiency) was quantified by UV absorbance at 215 nm (A₂₁₅) and/or 280 nm (A₂₈₀) using Equation 3 and Equation 4. Samples were diluted to be in the linear range of the UV detector in an appropriate diluent (Na-Bicarbonate for OVApep and 10 mM acetate, 150 mM NaCl, pH 7.0 for the Triple peptide) and the spectrophotometer was blanked against the corresponding diluent. The absorbance input (A input) and absorbance supernatant (A supernatant), (A supernatant=all washes combined) were determined by multiplication of the found A₂₁₅ value by the dilution factor and the pre-dilution volume, and were then used in Equation 3. The weight/weight (w/w) peptide to aluminum hydroxide [Al(OH)₃] ratio was calculated using Equation 4.

$$\begin{matrix}\% Peptide Adsorbed ( \% Binding Efficiency ) = \frac{( A input - A supernatant ) \times 100}{A input} & Equation 3 \\ Peptide \text{:} {Al ( OH )}_{3} ratio ( w \text{/} w ) = \frac{Weight Peptide input \times \% Peptide Adsorbed}{Weight {Al ( OH )}_{3} input \times 100} & Equation 4\end{matrix}$$

Amino Acid Analysis Procedure.

The concentrations of the peptide input and peptide supernatant solutions were determined by amino acid analysis (AAA) using standard procedures as performed by the Molecular Structure Facility (MSF Proteomics Core, University of California, Davis, USA). Briefly, samples were placed in 6N HCl acid under vacuum for 24 hrs at 110° C. The samples were then vacuum dried and brought up in a precise quantity of diluent containing Norleucine (NorLeu), as an internal standard, and injected onto the Hitachi 8800 amino acid analyzer. The amino acids are separated by strong cation exchange with a Transgenomic column and Pickering buffers, which increase in pH, ionic strength and temperature over the course of the run. The amino acids react with ninhydrin in a secondary reaction for detection in the visible wavelength. The peaks are identified and the amount of each amino acid is quantified using a standard curve in the same sequence as the samples. From the amount of amino acids present and known sequence, the amount of peptide is then calculated.

The % peptide adsorbed to aluminum hydroxide (% binding efficiency) was quantified using Equation 5. The weight input (W input) and weight supernatant (W supernatant) were determined by multiplication of the found concentration by the dilution factor and the pre-dilution volume, and were then used in Equation 5. The weight/weight (w/w) peptide to aluminum hydroxide ratio was calculated using Equation 4 above. The peptide concentrations found in the solutions were also correlated with the UV absorbance at 215 nm to determine extinction coefficients for the peptides at 215 nm.

$$\% Peptide Adsorbed ( \% Binding Efficiency ) = \frac{( W input - W supernatant ) \times 100}{W input} \qquad Equation 5$$

### Example S7: Production of OVApep:Aluminum Hydroxide:D61-01 Co-Adsorbates

This example describes methods of adsorption of CpG and antigen to aluminum hydroxide in various buffers.

### Experiment S7-1: Binding Using Aqueous Buffers

The co-adsorption of OVApep and D61-01 to aluminum hydroxide was achieved in two steps. In the first step, OVApep (5.4 mg) dissolved in 0.1 M Na-bicarbonate, pH 8.0 at a concentration of ˜1 mg/ml (w/v) was mixed by end-over-end rotation (100-150 rpms) with 1 ml (10 mg on an aluminum basis) of an aluminum hydroxide formulation (equilibrated in Na-Bicarbonate buffer) for 2 hrs at RT. OVApep was mixed with aluminum hydroxide at a level of approximately 50% of the predetermined adsorption capacity, thereby leaving ˜50% of the remaining surface area on aluminum hydroxide available for adsorption of D61-01 in a subsequent step. In the second step, ˜3.3 mg of D61-01 dissolved in 10 mM NaOAc, 150 mM NaCl, pH 7.0 at a concentration of ˜1.0 mg/mL was added to OVApep already adsorbed to aluminum hydroxide and again mixed by end-over-end rotation (100-150 rpms) for 2 hrs at RT. The final blended buffer was composed of approximately 50 mM Na-Bicarbonate, 5 mM NaOAc and 75 mM NaCl. After 3 cycles of washes using 0.1M Na-Bicarbonate and centrifugation of aluminum hydroxide to remove excess or weakly adsorbed OVApep and D61-01, the final amount of D61-01 and OVApep adsorbed to aluminum hydroxide was determined as described in Example S4 and Example S6 (UV procedure), respectively. In this experiment (S7-1) the binding efficiency of OVApep was only 54% in Na-bicarbonate, while 100% of D61-01 offered was bound (lot 11042015). The binding ratio found was OVApep:aluminum hydroxide:D61-01 of 0.3:1:0.3 (w/w/w) (Table S7-1).

### Experiment S7-2: Comparison of OVApep Binding Using Aqueous Buffer or a Combination of Isopropyl Alcohol and Aqueous Buffer in the Preparation of OVApep:Aluminum Hydroxide:D61-01 Co-Adsorbates

The binding of OVApep dissolved in either Na-Bicarbonate or 20% IPA/water to aluminum hydroxide was compared (Table S7-1). Surprisingly, the peptides dissolved in 20% IPA/water show higher binding efficiencies and binding capacities of peptide than when an all aqueous system was used. Specifically, when the OVApep was dissolved in 20% isopropyl alcohol (IPA) (lot 1104215-IPA) instead of Na-bicarbonate and similarly processed, the binding capacity of OVApep increased from 54% to 88% (and 100% of D61-01 offered was also bound) suggesting that this condition affected peptide adsorption to aluminum hydroxide. For this condition the binding ratio found was OVApep:aluminum:D61-01 of 0.7:1:0.7 (w/w/w).

TABLE S7-1
Co-adsorption of D61-01 and OVApep to Aluminum Hydroxide

| | | | | | | | | Found |
|---|---|---|---|---|---|---|---|---|
| | | | | | | D61- | D61- | OVApep:Al(OH)₃:D61- |
| Lot No. | Reaction | | OVApep | OVApep | OVApep | 01 | 01 | 01 |
| (peptide | volume | Al(OH)₃ | input | bound | binding | input | bound | Ratio |
| solvent) | ml) | (mg)¹ | (mg) | (mg) | efficiency² | (mg) | (mg) | (w/w/w)¹ |
| 11042015 (Na- | 10 | 10 | 5.4 | 2.9 | 54% | 3.3 | 3.3 | 0.3:1:0.3 |
| Bicarbonate)³ | | | | | | | | |
| 11042015 | 9 | 10 | 7.4 | 6.5 | 88% | 7.1 | 7.1 | 0.7:1:0.7 |
| (IPA)³ | | | | | | | | |

¹Weight of Al(OH)₃ is based on aluminum content
²Binding Efficiency (%) = {Peptide bound/Peptide input} × 100 (see Example S6)
³Peptide input was quantified by UV method in Example S6

### Experiment S7-3: Binding of OVApep and D61-01 to Aluminum Hydroxide in a Single Step

OVApep was dissolved in 20% IPA/water and the aluminum hydroxide and D61-01 were both present in a 10 mM NaOAc, 150 mM NaCl, pH 7.0 buffer. All components were added together in the ratios shown in Table S7-2 for 1 hr at RT. After combining the components, the final composition of the binding solution was ˜10% IPA in 5 mM NaOAc, 75 mM NaCl, pH 7.0 buffer. Under these conditions the peptide binding efficiency increased to 89% and 98% in this experiment for reactions at two different peptide input levels (5 and 10 mg), respectively, each reacted with 10 mg of aluminum hydroxide (based on aluminum content). Surprisingly, the binding of 0.9 mg of OVApep per mg of aluminum hydroxide did not significantly diminish the binding capacity of the D61-01, which also bound with a ratio of 0.9 mg per mg of aluminum hydroxide, similar to the binding capacity observed for D61-01 alone in Example S3, Experiment S3-2. The ability to bind high levels of both peptide and CpG-ODN to aluminum hydroxide is advantageous since it allows maximum dosing of peptide and CpG-ODN using minimal aluminum hydroxide as carrier.

TABLE S7-2
Co-adsorption of D61-01 and OVApep onto Aluminum Hydroxide in a Single Reaction

| | | | | | | | | OVApep:Al(OH)₃:D61- |
|---|---|---|---|---|---|---|---|---|
| | Reaction | | OVApep | OVApep | OVApep | D61-01 | D61-01 | 01 |
| | volume | Al(OH)₃ | input | bound | binding | input | bound | Ratio |
| Lot No. | (ml) | (mg)¹ | (mg) | (mg) | efficiency² | (mg) | (mg) | (w/w/w)¹ |
| 11302015 (1) | 7.1 | 10 | 5.0 | 4.9 | 98% | 4.6 | 4.6 | 0.5:1:0.5 |
| 11302015 (2) | 14.2 | 10 | 10.0 | 8.9 | 89% | 9.2 | 9.1 | 0.9:1:0.9 |

¹Weight of Al(OH)₃ is based on aluminum content
²Binding Efficiency (%) = {Peptide bound/Peptide input} × 100 by UV method (see Example S6)

### Experiment S7-4: Preparation of Additional OVApep:Aluminum Hydroxide:D61-01 Co-Adsorbates for Use in Biological Studies

In this two-step procedure OVApep (two different samples) was dissolved in 0.1 M sodium bicarbonate, pH 8.0 (lot 09292105(3)) and the aluminum hydroxide was equilibrated in sodium bicarbonate, pH 8.0 buffer. D61-01 was dissolved in 10 mM NaOAc, 150 mM NaCl, pH 7.0. For lot 0331-2015, the aluminum hydroxide was equilibrated in sodium acetate. First, the OVApep at 1-2 mg/mL was added to aluminum hydroxide (10 mg based on aluminum) and mixed end-over-end for 2 hrs at RT. The binding results for OVApep were moderate at 51% and 56%, respectively as shown in Table S7-3. Second, D61-01 at 2-4 mg/mL was added and mixed for 1 hr at RT. The aluminum hydroxide complex was washed extensively with sodium bicarbonate buffer after both peptide adsorption and D61-01 adsorption steps for both lots. The D61-01 binding efficiency was 53% for lot 0331-2105 and 100% for lot 09292105(3) as shown in Table S7-3.

TABLE S7-3
Summary of Co-adsorption of OVApep and D61-01 to Aluminum Hydroxide

| | | | | | | D61- | | OVApep:Al(OH)₃:D61- |
|---|---|---|---|---|---|---|---|---|
| | Reaction | | | OVApep | OVApep | 01 | D61-01 | 01 |
| | volume | Al(OH)₃ | OVApep | bound | binding | input | bound | Ratio |
| Lot No. | (mL) | (mg)¹ | input (mg) | (mg) | efficiency | (mg) | (mg) | (w/w/w)¹ |
| 0331-2015 | 5 | 10 | 6.1 | 3.1 | 51% | 8.1 | 4.3 | 0.6:1:0.9 |
| 09292015 (3) | 10 | 10 | 3.9 | 2.2 | 56% | 2.2 | 2.2 | 0.2:1:0.2 |

¹Weight of Al(OH)₃ is based on aluminum content
²Binding Efficiency (%) = Peptide bound/Peptide input × 100 by UV method (see Example S6)

### Example S8: Production of Triple Peptide:Aluminum Hydroxide:D61-01 Co-adsorbates

The two-step procedure was used to adsorb the Triple peptide and D61-01 to aluminum hydroxide. First, the Triple peptide was dissolved in 20% IPA/water at a 1-2 mg/ml and was adsorbed to aluminum hydroxide in 10% IPA, 5 mM NaOAc, 75 mM NaCl, pH 7.0 buffer for 2 hrs at 20-25° C. Second, D61-01 dissolved at a concentration of 2-4 mg/mL in 10 mM NaOAc, 150 mM NaCl, pH 7.0 buffer was added and mixed for 1 hr at 20-25° C.

The Triple peptide and D61-01 were both efficiently co-adsorbed to aluminum hydroxide at two different scales in the presence of IPA, as shown in Table S8-1. Binding efficiencies for both ligands were high i.e, >87%. Both reactions showed close to a 1:1:1 ratio for Triple peptide:aluminum hydroxide (based on aluminum content):D61-01 (w/w/w) and demonstrated the process is reproducible.

TABLE S8-1
Summary of Co-adsorption of Triple Peptide and D61-01 to Aluminum Hydroxide

| | | | | | | D61- | D61- | | Found |
|---|---|---|---|---|---|---|---|---|---|
| | Reaction | | | Triple | Triple | 01 | 01 | D61-01 | Triple:AL(OH)₃:D61- |
| | volume | | Triple | bound | binding | input | bound | binding | 01 |
| Lot No. | (mL) | Al(OH)₃ (mg)¹ | input (mg) | (mg) | efficiency² | (mg) | (mg) | efficiency | (w/w/w)¹ |
| 10012015 | 16 | 10 | 11 | 10 | 91% | 10 | 9 | 90% | 1.0:1:0.9 |
| 10142015 | 36 | 20 | 24 | 23 | 96% | 23 | 20 | 87% | 1.1:1:1.0 |

¹Weight of Al(OH)₃ is based on aluminum content
²% Binding Efficiency (%) = {Peptide bound/Peptide input} × 100 by UV method (see Example S6)

### Experiment S8-2: Binding of a Constant Mass of D61-04 ODN to a Varying Mass of Co-Adsorbed NY-ESO-1 OLP Antigens-Aluminum Hydroxide Particles, and Displacement of Co-adsorbed OLP Antigens

The ability of a constant mass of D61-04 polynucleotide to bind the various co-adsorbed NY-ESO-1₇₉₋₁₀₈ and NY-ESO-1₁₄₂₋₁₇₃ OLP antigens-aluminum hydroxide particles from Experiment S5-3 was assessed to determine 1) the binding of D61-04 and 2) the extent of NY-ESO-1₇₉₋₁₀₈ and NY-ESO-1₁₄₂₋₁₇₃ OLP antigen displacement. Neat D61-04 polynucleotide was dissolved at 1 mg/mL in 10 mM NaOAc, 150 mM NaCl, pH 7.0 and the exact concentration was determined by the UV absorbance method described in Example S4. Then, a 1:1 (v/v) solution containing 1 mg of D61-04 polynucleotide in 10 mM NaOAc, 150 mM NaCl, pH 7.0 and a solution containing the 5 different OLP antigens-aluminum hydroxide particles in 10 mM NaOAc, 150 mM NaCl, pH 7.0 were mixed in an end-over-end mixer at ˜15 rpm for ˜3 hrs at 20-25° C. Next unbound D61-04 polynucleotide, and displaced OLP antigens, were separated from co-adsorbed D61-04 polynucleotide—OLP antigens-aluminum hydroxide particles, by centrifugation as described in Experiment S5-3. The washing/centrifugation process was repeated two more times with the same buffer used for binding, and the pooled supernatants were analyzed for unbound D61-04 polynucleotide by the UV absorbance method, and for displaced OLP antigens by amino acid analysis (see Example S6). A linear relationship was observed between increasing mass of co-adsorbed NY-ESO-1₇₉₋₁₀₈ and NY-ESO-1₁₄₂₋₁₇₃ OLP antigen-aluminum hydroxide particles and the % binding of a fixed input mass of D61-04 polynucleotide (Table S5-4). In contrast, no further displacement of the NY-ESO-1₇₉₋₁₀₈ and NY-ESO-1₁₄₂₋₁₇₃ OLP antigens was observed except a slight 10% displacement in the 0.6 mg aluminum hydroxide particle condition.

TABLE S8-2
Adsorption of D61-04 to, and Displacement of NY-ESO-1 OLP Antigens from OLP
Antigen - Aluminum Hydroxide Particles in 10 mM NaOAc, 150 mM NaCl pH 7.0 Buffer

| | Al(OH)₃ | NY-ESO-1_{79 - 108 +} | | | |
|---|---|---|---|---|---|
| | Input | NY-ESO-1₁₄₂₋₁₇₃ | D61-04 Input | D61-04 | OLP % |
| Lot No. | (mg)¹ | Co-adsorbed (mg)² | (mg)³ | % bound⁴ | displacement⁵ |
| CC-100316-01 | 1.0 | 0.19 | 1.0 | 80 | 0 |
| CC-100316-02 | 0.8 | 0.19 | 1.0 | 60 | 0 |
| CC-100316-03 | 0.6 | 0.18 | 1.0 | 50 | 16 |
| CC-100316-04 | 0.4 | 0.16 | 1.0 | 30 | 0 |
| CC-100316-05 | 0.2 | 0.16 | 1.0 | 20 | 0 |

¹Mass of Al(OH)₃ is based on input aluminum content
²Mass of co-adsorbed NY-ESO-1 OLPs on aluminum hydroxide particles by amino acid analysis
³Mass of input D61-04 polynucleotide by UV analysis method
⁴Percent (%) binding = {polynucleotide bound/polynucleotide input} × 100, by UV analysis method
⁵Percent (%) displacement = (1 − {peptide bound after D61-04 binding/peptide bound before D61-04 binding}) × 100, by amino acid analysis method

### Experiment S8-3: Binding of a Constant Mass of D61-04 to Co-Adsorbed NY-ESO-1 OLP Antigens-Aluminum Hydroxide Particles with Increasing OLP Mass, and Displacement of Co-Adsorbed OLP Antigens

The ability of a fixed quantity of D61-04 polynucleotide to bind various NY-ESO-1₇₉₋₁₀₈ and NY-ESO-1₁₄₂₋₁₇₃OLP-aluminum hydroxide particles from Experiment S5-5 was assessed to determine 1) D61-04 binding and 2) the extent of NY-ESO-1₇₉₋₁₀₈ and NY-ESO-1₁₄₂₋₁₇₃ OLP displacement. Neat D61-04 polynucleotide was dissolved in 10 mM NaOAc, 150 mM NaCl, pH 7.0 and the exact concentration was determined by the UV absorbance method described in Example S4. Then, a 1:1 (v/v) ratio of a slurry containing 1 mg of D61-04 polynucleotide in 10 mM NaOAc, 150 mM NaCl, pH 7.0 and a solution containing the 6 different ratios of co-adsorbed OLPs on aluminum hydroxide particles in 10 mM NaOAc, 150 mM NaCl, pH 7.0 were mixed in an end-over-end mixer at ˜15 rpm for ˜3 hrs at 20-25° C. Next unbound D61-04 polynucleotide, and displaced aluminum hydroxide co-adsorbed OLPs, were separated by centrifugation as described above. The washing/centrifugation process was repeated two more times with the same buffer used for binding, and the pooled supernatants were analyzed for unbound D61-04 polynucleotide by the UV absorbance method and displaced OLP by amino acid analysis (see Example S6). A linear relationship was observed between increasing mass of NY-ESO-1₇₉₋₁₀₈ and NY-ESO-1₁₄₂₋₁₇₃ OLP-aluminum hydroxide particles and increasing amounts of co-adsorbed D61-04 mass (Table S5-3). In contrast, no to minimal displacement of the 0.2, 0.25, 0.3, 0.4, 0.5 or 0.6 mgs total OLPs (0.1, 0.125, 0.15, 0.2, 0.25 or 0.3 mgs of each OLP) of the combined NY-ESO-1₇₉₋₁₀₈ and NY-ESO-1₁₄₂₋₁₇₃ OLPs was observed.

TABLE S8-3
Co-adsorption of D61-04 to, and Displacement of NY-ESO-1 OLPs from,
Aluminum Hydroxide Particles in 10 mM NaOAc, 150 mM NaCl, pH 7.0 Buffer

| | Al(OH)₃ | NY-ESO-1_{79 - 108 +} | | | |
|---|---|---|---|---|---|
| | Input | NY-ESO-1₁₄₂₋₁₇₃ | D61-04 Input | D61-04 | % OLP |
| Lot No. | (mg)¹ | Co-adsorbed (mg)² | (mg)³ | % bound⁴ | displacement⁵ |
| CC-261016-05 | 1.0 | 0.18 | 1.0 | 82 | 0 |
| CC-261016-03 | 1.0 | 0.23 | 1.0 | 82 | 0 |
| CC-261016-01 | 1.0 | 0.27 | 1.0 | 86 | 0 |
| CC-261016-06 | 0.5 | 0.16 | 1.0 | 48 | 6 |
| CC-261016-04 | 0.5 | 0.23 | 1.0 | 48 | 0 |
| CC-261016-02 | 0.5 | 0.27 | 1.0 | 48 | 0 |

¹Mass of Al(OH)₃ is based on input aluminum content
²Mass of co-adsorbed NY-ESO-1 OLPs on aluminum hydroxide particles by amino acid analysis
³Mass of input D61-04 polynucleotide by UV analysis method
⁴Percent (%) bound = {polynucleotide bound/polynucleotide input} × 100, by UV method
⁵Percent (%) displacement = (1 − {peptide bound after D61-04 binding/peptide bound before D61-04 binding}) × 100, by amino acid analysis method

### Example S9: Preparation of CpG-FICOLL-Peptide Conjugates

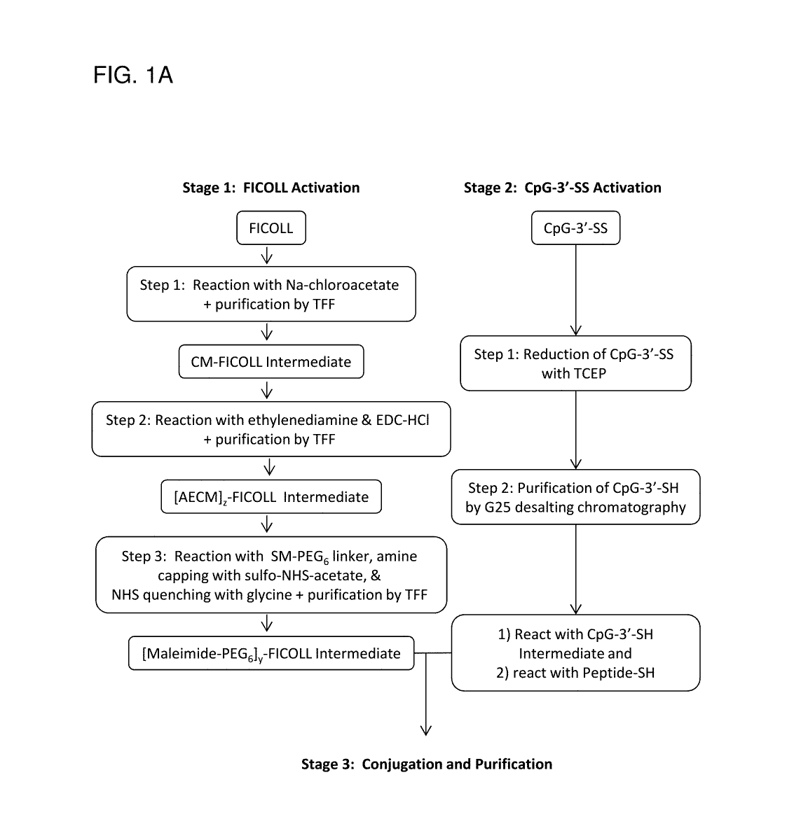

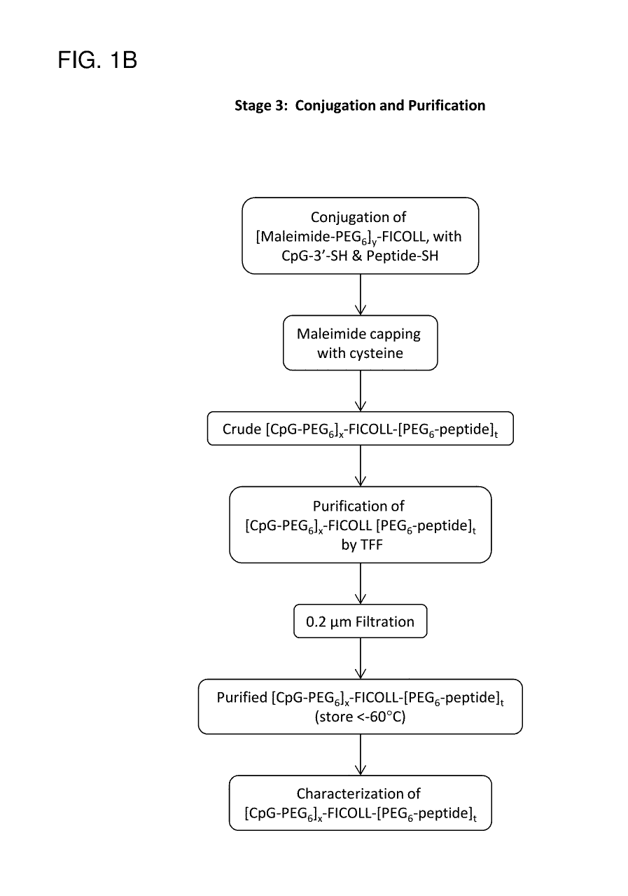

The manufacturing scheme for CpG-FICOLL-Peptide Conjugates is provided in FIG. 1A-B. In this example, the polysaccharide multimerization agent was a high MW, branched copolymer of sucrose and epichlorohydrin marketed as FICOLL® marketed by GE Healthcare. However, generic versions or biosimilars (unbranded or other brands) are also suitable for use. Other CpG-ODN or peptide conjugates to FICOLL® can be prepared by the same manufacturing route by changing the CpG-ODN and/or peptide sequence, the thiol linker to the PN or CC CpG-ODN, and/or the thiol to amine crosslinker.

In Stage 1, FICOLL is modified in several steps to include a reactive maleimide group, resulting in [Maleimide-PEG₆]ₓFICOLL. In Stage 2, the disulfide in the exemplary CpG-ODN, D61-02 (aka (D61-01)-3′-SS), is reduced to a thiol, forming D61-03 (aka (D61-01)-3′-SH). In Stage 3, [Maleimide-PEG₆]_{y}-FICOLL, D61-03 and cysteine-modified peptide react to form CpG-FICOLL-peptide (in this case D61-01-FICOLL-peptide). Purification occurs at each step in the process. The final CpG-FICOLL-peptide solution is sterile filtered and characterized, before storage at <−60° C. Molar ratio results provided in the tables of examples for the CpG-FICOLL-Peptide conjugates are average molar ratios (average loading ratios on a molar basis).

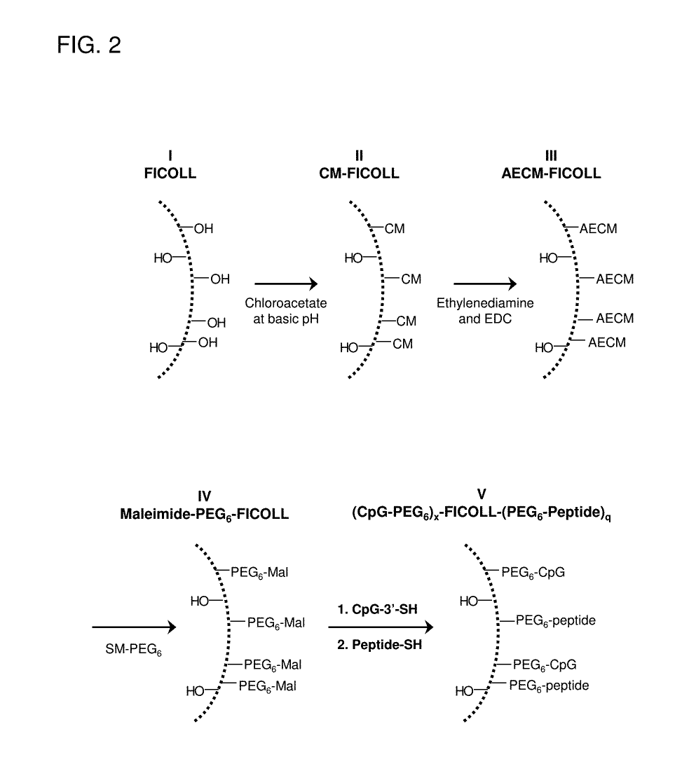

FIG. 2 outlines the process for manufacture of the FICOLL intermediates carboxymethylated (CM)-FICOLL, aminoethylcarbamylmethylated [AECM]_{z}-FICOLL, and [Maleimide (Mal)-PEG₆]_{y}-FICOLL, and the final product (CpG-ODN-PEG₆)ₓ-FICOLL-(PEG₆-Peptide),

### A. Composition of FICOLL PM400.

FICOLL PM400 (FICOLL₄₀₀) is a synthetic neutral, highly-branched polymer of sucrose with a reported molecular weight of 400,000±100,000 that exists as a suspension of nanoparticles. It is formed by copolymerization of sucrose with epichlorohydrin. FICOLL PM400 was purchased as a spray dried powder from GE Healthcare (Pittsburgh, Pa.).

B. Preparation of [Maleimide (Mal)-PEG₆]_{y}-FICOLL.

[Maleimide (Mal)-PEG₆]_{y}-FICOLL was prepared as shown schematically in FIG. 2 and previously described (see, PCT patent application PCT/US2016/014635, filed Jan. 22, 2016).

Briefly, CM-FICOLL was prepared from FICOLL PM400 and sodium chloroacetate under basic conditions by the method of Inman (J Immunol, 114:704-709, 1975), except that instead of using a standard desalting procedure such as via dialysis using a 5 kDa molecular weight cut-off (MWCO) membrane), purification using tangential flow fractionation (TFF) with a 100 kDa MWCO membrane was performed. The TFF purification removed molecules and excess reagents similarly to the standard desalting procedure.

[AECM]_{z}-FICOLL was prepared from CM-FICOLL with a large excess of ethylenediamine and a water soluble carbodiimide by the method of Inman (supra), modified to employ a purification step using tangential flow fractionation (TFF) with a 100 kDa MWCO membrane.

[Maleimide-PEG₆]_{y}-FICOLL was prepared by reaction of [AECM]_{z}-FICOLL (20 mg/mL in 100 mM sodium phosphate and 150 mM sodium chloride, pH 7.5 buffer, amine to FICOLL molar ratio (z)=218-224) with SM-PEG₆ (100 mg/mL in dimethylsulfoxide, 5 equivalents per amine) for 40 min at RT. SM-PEG₆ (succinimidyl-((N-maleimidopropionamido)-hexethyleneglycol) ester) was obtained from Thermo Scientific of Rockford, Ill. (Catalog No. 22105). Unreacted amines on the FICOLL were capped using sulfo-N-hydroxysuccinimidyl-acetate (Su-NHS-Ac) from Thermo Scientific (100 mg/mL in dimethyl sulfoxide, 5 equivalents per amine) for 15 min at RT. This capping reaction converts the unreacted amines on the FICOLL to acetamides, which may be important for the physicochemical properties of the resulting FICOLL product. Unreacted SM-PEG₆ and Su-NHS-Ac were quenched with glycine (100 mg/mL in 100 mM sodium phosphate and 150 mM sodium chloride, pH 7.5 buffer, 10 equivalents per total of SM-PEG₆ and Su-NHS-Ac) for 15 min at RT. The crude [Maleimide-PEG₆]_{y}-FICOLL was purified on the same day as the conjugation reaction by TFF with a 100 kDa MWCO membrane. The crude [Maleimide-PEG₆]_{y}-FICOLL was diluted to about 5.8 mg/mL using 100 mM sodium phosphate, 150 mM sodium chloride, pH 7.5 buffer, and was diafiltered against 100 mM sodium phosphate, 150 mM sodium chloride, pH 7.5 buffer for a total of approximately 24-29 volume exchanges. The absorbance of each permeate diavolume was measured at 215 nm and the diafiltration was stopped when the permeate absorbance reached 0.1 AU. The purified [Maleimide-PEG₆]_{y}-FICOLL was aliquoted into sterile polypropylene vials and stored at −80° C.

The maleimide to FICOLL molar ratio (y) of [Maleimide-PEG₆]_{y}-FICOLL was determined by the procedures outlined in Example S10 and Example S11. Table S9-1 shows the exemplary batches of [Maleimide-PEG₆]_{y}-FICOLL produced and conjugates that were formed from them (see section D).

TABLE S9-1
Exemplary Batches of [Maleimide-PEG₆]_{y}-FICOLL
Produced and Conjugates Prepared

| [Maleimide-PEG₆]_{y}- | Maleimide:FICOLL | |
|---|---|---|
| FICOLL Lot No. | molar ratio (y) | Conjugate description |
| 07262012 | 151 | (D61-01)-FICOLL-OVA, |
| | | lot 01132014B |
| 08252014 pool#2 | 155 | (D61-01)-FICOLL-OVApep, |
| | | lot 04082015 |
| 04172013 | 206 | (D61-01)-FICOLL-OVApep, |
| | | lot 04302014 |
| 09102015 | 182 | (D61-01)-FICOLLL-OVApep, |
| | | lot 09232015 |
| 08252014 pool#2 | 155 | (D61-01)-FICOLL-Triple, |
| | | lot 04142015 |

### C. Preparation of D61-03 (aka (D61-01)-3′-SH).

D61-03 was prepared as previously described (see, PCT patent application PCT/US2016/014635, filed Jan. 22, 2016). Briefly, D61-02 (aka (D61-01)-3′-SS, 56 mg/mL in activation buffer) was reacted with TCEP-HCl (Thermo Scientific, Rockford, Ill., 48 mg/mL in activation buffer, 5 equivalents) at about 40° C. for about 2 hrs. Activation buffer was 100 mM sodium phosphate, 150 mM sodium chloride, 1 mM ethylenediaminetetraacetic acid (EDTA), pH 7.5. The crude D61-03 (aka (D61-01)-3′-SH) was purified by gel filtration using Sephadex G-25 Fine (GE Healthcare, Pittsburgh, Pa.) packed into XK50/30 columns (GE Healthcare) according to the manufacturer's recommended procedures and using 100 mM sodium phosphate, 150 mM sodium chloride, 1 mM EDTA, pH 7.5 buffer as the mobile phase. The eluent was monitored at 215 nm and 260 nm and the purified fractions were combined, aliquoted and stored at −80° C.

### D. Preparation of (D61-01)-FICOLL-Peptide Co-Conjugates.

In a typical synthesis of CpG-FICOLL-peptide, the activated CpG-ODN (e.g., D61-03, aka (D61-01)-3′-SH) is added and mixed with Mal-FICOLL for about 15 min prior to the addition of the peptide. After 15 min, solid guanidine hydrochloride (GuHCl) is added to the mixture to achieve a ˜6M final concentration, followed by the solid peptide. The GuHCl is necessary to solubilize the peptide and enable covalent linkage via the cysteine group (thiol) with accessible maleimides on FICOLL. A detailed example of the procedure is provided in the following paragraphs and is also applicable for other CpG-ODN and peptide sequences.

Details of Exemplary Procedure for Producing a D61-01-FICOLL-Triple Melanoma (Triple) Peptide Co-Conjugate, Lot 04142015.

Approximately 44 milligrams (mg), (8.1 ml) of Maleimide-FICOLL (lot 08252014), at a FICOLL concentration of 5.4 mg/ml (0.00011 mmol FICOLL, 0.017 mmol maleimide or 155 maleimide per FICOLL) in 100 mM sodium phosphate, 150 mM sodium chloride, pH 7.5 buffer was added to a 15 ml tube and reacted with 25.7 mg (2.1 ml, 30 equivalents per FICOLL) of 3′thiol D61-01 (lot 02262014) at concentration of 12.2 mg/ml in 100 mM sodium phosphate, 150 mM sodium chloride, 1 mM EDTA, pH 7.5 buffer. This reaction (˜10.3 ml) proceeded for 15 min in a 25° C. incubator. Next, approximately 5.9 grams of solid GuHCl was added to solution to achieve a final GuHCl concentration of approximately 6 M. This solution was mixed by vortexing for about 1 min until the GuHCl was in solution, then 28 mg (40 equivalents per FICOLL) of solid Triple peptide (lot P2345-1 from Bio-Synthesis) was immediately added and vortexed until dissolved. The mixture was allowed to react for 45-60 min at 25° C. After the reaction, potential unreacted maleimides were capped with cysteine (Thermo; Catalog No. 44889). Briefly, about 0.21 ml of a 100 mg/ml stock solution of cysteine (21 mg, 0.17 mmol, 10 equivalents per maleimide) was added and maintained at RT (25° C.) for 25 min. The final crude co-conjugate (about 14.5 ml) composition contained approximately 2.8 mg/ml of FICOLL, 1.6 mg/ml of 3′thiol D61-01, 1.8 mg/ml of Triple peptide and 1.3 mg/ml of cysteine and was stored overnight at 2-8° C. before purification. The resulting co-conjugate was purified by size-exclusion chromatography using HiLoad 16/600 mm Superdex 200 prep grade column (GE Healthcare) with an about 120 ml bed volume. The column was equilibrated and run isocratically in 10 mM sodium phosphate, 142 mM NaCl, pH7.2 at a flow rate of 1 ml/min at RT. The purification was controlled using an Akta Purifier (GE Healthcare) chromatography system. Approximately 14 ml of crude sample was applied to the column and the total run time was 150 min. Purification was monitored by UV detection at absorbances of 215, 260 and 280 nm and the purified D61-01-FICOLL-Triple peptide co-conjugate (lot 04142015) was isolated in a volume of about 18 ml and subsequently characterized (Table S9-2). The D61-01-FICOLL conjugate was prepared as described above except that the GuHCl and peptide solution were not added.

TABLE S9-2
Characterization of D61-01-FICOLL Conjugates and
DV61-01-FICOLL-Peptide Co-conjugates by Lot Number

| | | D61-01- | D61-01- | D61-01- | D61-01- | D61-01- |
|---|---|---|---|---|---|---|
| | D61-01- | FICOLL- | FICOLL- | FICOLL- | FICOLL- | FICOLL- |
| | FICOLL | OVA¹ | OVApep | OVApep | OVApep | Triple |
| Attributes | (10142011) | (01132014B) | (04082015) | (04302015) | (09232015) | (04142015) |
| Appearance | clear | clear | clear | clear | clear | clear |
| | liquid | liquid | liquid | liquid | liquid | liquid |
| pH | 7.3 | 7.2 | 7.2 | 7.2 | 7.2 | 7.2 |
| Purity by | >95% | 100% | 100% | >99% | 100% | 100% |
| SEC-HPLC² | | | | | | |
| FICOLL | 1.3 | 0.32 | 1.7 | 1.6 | 1.6 | 1.7 |
| (mg/ml)³ | | | | | | |
| Peptide | NA | 0.37 | 1.1 | 0.8 | 1.1 | 0.9 |
| by AAA | | | | | | |
| (mg/ml)⁴ | | | | | | |
| Peptide to | NA | 11 | 72 | 52 | 71 | 35 |
| FICOLL | | | | | | |
| molar ratio | | | | | | |
| D61-01 by | 2.9 | 0.56 | 0.98 | 1.0 | 0.9 | 1.2 |
| A260 | | | | | | |
| (mg/ml)⁵ | | | | | | |
| D61-01 to | 117 | 93 | 30 | 34 | 27 | 36 |
| FICOLL | | | | | | |
| molar ratio | | | | | | |

¹Ova refers to Ovalbumin protein
²Purity by SEC-HPLC determined by procedure in Example S14
³Ficoll content determined by procedure in Example S10
⁴Peptide content determined by procedure in Example S6 (AAA)
⁵D61-01 content determined by procedure in Example S4

Additional co-conjugates were prepared in a similar manner as described above and their results are provided in Table S9-3 and Table S9-4.

TABLE S9-3
Characterization of D61-01-FICOLL-Triple Co-conjugates

| | | Found | | Found | # of | Found |
|---|---|---|---|---|---|---|
| | Peptide | Peptide:FICOLL | CpG | CpG:FICOLL | Mal:PEG6- | Peptide:FICOLL:CpG |
| Lot No. | target | molar ratio | target | molar ratio | FICOLL | molar ratios |
| 10072015 | 40 | 33 | 12 | 16 | 182 | 33:1:16 |

TABLE 9-4
Characterization of D61-01-FICOLL-OVApep Co-conjugates

| | | Found | | Found | |
|---|---|---|---|---|---|
| | Peptide | Peptide:FICOLL | | CpG:FICOLL | # of |
| Lot No. | target | molar Ratio | CpG target | molar Ratio | Mal:PEG₆-Ficoll |
| 07102015 | 89 | 67 | 27 | 27 | 155 |
| 09012015 | 89 | 65 | 27 | 25 | 155 |
| 09232015 | 89 | 71 | 27 | 27 | 182 |

### Example S10: Procedure to Determine FICOLL Concentration in FICOLL-Containing Intermediates and Products

The FICOLL concentrations of FICOLL-containing intermediates and products were determined using the Pierce Glycoprotein Carbohydrate Estimation Kit (Product No. 23260, Thermo Scientific, Rockford, Ill.) as per the manufacturer's protocol, except that FICOLL PM400 was used to create a standard curve for the assay.

### Example S11: Procedure to Determine Maleimide Concentration and Maleimide:FICOLL Molar Ratio (y) in [Maleimide]_{y}-FICOLL Solutions

The maleimide concentrations of [Maleimide-PEG₆]_{y}-FICOLL were determined using Ellman's reagent (5,5′-dithio-bis-(2-nitrobenzoic acid), Product No. 22582, Thermo Scientific, Rockford, Ill.). The [Maleimide-PEG₆]_{y}-FICOLL was reacted with excess cysteine as per the manufacturer's protocol, and the remaining cysteine was quantified using a cysteine standard curve. The maleimide concentration was determined by subtracting the remaining cysteine concentration from the initial cysteine concentration. The Maleimide:FICOLL molar ratio (y) was calculated by dividing the maleimide concentration by the FICOLL concentration, where the FICOLL concentration was determined as described in Example S4 and the concentrations were in units of molarity.

### Example S12: Procedure to Determine CpG Concentration and CpG:FICOLL Molar Ratio (x) in Conjugates

The D61-01 concentration of (D61-01)-FICOLL-Peptide was determined using ultraviolet spectrophotometry and the Beer's law equation. By convention, the compound attached to the FICOLL is referred to by the sequence name, D61-01, at this stage even though the chimeric compound with the linker, D61-03, was used to form this compound. The absorbance at 260 nm was determined and an extinction coefficient of 22.65 mg/ml⁻¹×cm⁻¹ for D61-01 was used. FICOLL, the linkers and the peptides do not absorb at 260 nm, so the absorbance is solely due to the absorbance of the CpG-ODN, D61-01. The D61-01 concentration in mg/mL was converted to a molar concentration using the molecular weight of the free acid for D61-01. The CpG:FICOLL molar ratio (x) was determined by dividing the CpG-ODN concentration by the FICOLL concentration, where the FICOLL concentration was determined as described in Example S10 and the concentrations were in units of molarity. Concentrations for other CpG-FICOLL solutions are determined using the extinction coefficient and free acid molecular weight for the CpG-ODN used, as appropriate.

### Example S13: Procedure to Determine Particle Size

The particle sizes (Z-average) and standard deviations (SD) of FICOLL samples (e.g., CpG-FICOLL-peptide) were measured by dynamic light scattering (DLS) using a Malvern Zetasizer instrument. Samples were diluted to a FICOLL concentration of 0.5 mg/mL in 10 mM sodium phosphate, 142 mM sodium chloride, pH 7.2 buffer, and measured under defined instrument settings. A calibrated 50 nm polystyrene nanosphere sample (Product No. 3050A, Thermo Scientific, Rockford, Ill.) was included in the analysis as a system suitability control and had had a particle size of 49±6 nm.

### Example S14: Procedure to Determine Purity by SEC-HPLC

The HPLC parameters to determine percentage purity (by area) by SEC-HPLC are provided in Table S14-1.

TABLE S14-1
SEC-HPLC Method For Purity Determination

| Column | TOSOH TSK-Gel G3000 PW_{XL} |
|---|---|
| Dimensions | 7.8 mm × 30 cm |
| Bed Volume | 14.3 ml |
| Flow Rate | 0.75 ml/min |
| Mobile Phase | 10 mM sodium phosphate, 141.7 mM NaCl, |
| | pH 7.2 buffer |
| Run Time | 20 min |
| Detection | UV at 215 and 260 nm |
| Injection Volume | 20 μl |

### Example S15: Binding of HPV16 Peptide and D61-01 to Aluminum Hydroxide

HPV16 peptide (SEQ ID NO: 19) was dissolved in 20% IPA/water (˜1-2 mg/ml) and reacted for 2 hrs with aluminum hydroxide (equilibrated in 10 mM NaOAc, 150 mL NaCl, pH 7.0). After combining the components, the final composition of the binding solution was about 10% IPA in 5 mM NaOAc, 75 mM NaCl, pH 7.0. After adsorbing peptides to aluminum hydroxide complex, the complex was washed twice in 10 mM NaOAc, 150 mM NaCl, pH 7.0 to remove non-adsorbed peptide, and the moist gel was held overnight at 2-8° C. The next day, D61-01 also dissolved in 10 mM NaOAc, 150 mL NaCl (about 1-2 mg/mL) was added to HPV16 peptide already bound to aluminum hydroxide, reacted for 1 hr by end-over-end mixing (100-150 rpm), and again washed twice in 10 mM NaOAc, 150 mL NaCl, pH 7.0. Details of the amounts of D61-01, HPV16 peptide and aluminum hydroxide used in each reaction are provided in Table S15-1, along with the binding efficiencies. Under these conditions the peptide binding efficiency was high, at 90% and 83% for the two binding reactions. The D61-01 binding efficiencies were 74% and 80% for the two reactions.

TABLE S15-1
Co-adsorption of D61-01 and HPV16 peptide onto Aluminum Hydroxide

| | Reaction | | Peptide | Peptide | Peptide | D61-01 | D61-01 | Peptide:AL(OH)₃:D61- |
|---|---|---|---|---|---|---|---|---|
| | volume | Al(OH)₃ | input | bound | binding | input | bound | 01 Ratio |
| Lot No. | (ml) | (mg)¹ | (mg) | (mg) | efficiency² | (mg) | (mg) | (w/w/w)¹ |
| 11122015 | 48 | 20 | 30 | 27 | 90% | 23 | 17 | 1.4:1:0.9 |
| 02162016 | 19 | 10 | 12 | 10 | 83% | 10 | 8 | 1:1:0.8 |

¹Weight of Al(OH)₃ based on aluminum content
²Binding Efficiency (%) = {Peptide bound/Peptide input} × 100 (Example S6 by UV)

### Example S16: Binding of AH1 CII Peptide and D61-01 to Aluminum Hydroxide

The binding capacities of AH1 CII peptide (SEQ ID NO:26) and D61-01 to aluminum hydroxide was evaluated by varying the amount of aluminum hydroxide while holding the amounts of AH1 CII peptide and D61-01 offered constant, as shown in Table S16-1. The AH1 CII peptide was dissolved in 20% IPA/water (1-2 mg/ml) and reacted for 2 hrs with aluminum hydroxide (equilibrated in 10 mM NaOAc, 150 mL NaCl, pH 7.0). After combining the components, the final composition of the binding solution was ˜10% IPA in 5 mM NaOAc, 75 mM NaCl, pH 7.0. After adsorbing peptides to aluminum hydroxide complex, the complex was washed twice in 10 mM NaOAc, 150 mM NaCl, pH 7.0 to remove non-adsorbed peptide, and held overnight at 2-8° C. The next day, D61-01 also dissolved in 10 mM NaOAc, 150 mL NaCl (1-2 mg/mL) was added to AH1CII peptide already bound to aluminum hydroxide, reacted for 1 hr by end-over-end mixing (100-150 rpm), and again washed twice in 10 mM NaOAc, 150 mL NaCl, pH 7.0. Under these conditions the AH1 CII peptide binding efficiencies were high, ranging from 84% to 89%, and D61-01 binding was 100% in four out of five conditions, and 86% for the remaining condition (2.5 mg of aluminum), as shown in Table S16-1. These data show that a wide-range of ligand binding distributions (peptide:aluminum hydroxide:D61-01 ratios) can easily be produced.

TABLE S16-1
Co-adsorption of D61-01 and AH1 CII Peptide onto Aluminum Hydroxide

| | Reaction | | | | | | | Peptide:Al(OH)₃:D61- |
|---|---|---|---|---|---|---|---|---|
| | volume | | Peptide | Peptide | Peptide | D61-01 | D61-01 | 01 |
| | (ml) | Al(OH)₃ | input | bound | binding | input | bound | Ratio |
| Lot No. | peptide | (mg)¹ | (mg) | (mg) | efficiency² | (mg) | (mg) | (w/w/w)¹ |
| 02242016-1 | 7 | 30 | 5 | 4.4 | 89% | 5 | 5 | 0.2:1:0.2 |
| 02242016-2 | 7 | 20 | 5 | 4.4 | 89% | 5 | 5 | 0.2:1:0.2 |
| 02242016-3 | 7 | 10 | 5 | 4.2 | 84% | 5 | 5 | 0.4:1:0.5 |
| 02242016-4 | 7 | 5 | 5 | 4 | 80% | 5 | 5 | 0.8:1:1 |
| 02242016-5 | 7 | 2.5 | 5 | 4.2 | 84% | 5 | 4.3 | 1.7:1:1.7 |

¹Weight of Al(OH)₃ is based on aluminum content
²Binding Efficiency (%) = {Peptide bound/Peptide input} × ′100 (Example S6 by UV)

### Example B1: Effect on Tumor Size of Intratumoral Versus Extratumoral Administration of Immunogenic Compositions

This example describes the control of tumor growth by administration of immunogenic compositions comprising particles comprising a TLR9 agonist (CpG) associated with a polysaccharide multimerization agent alone or in further association with a tumor antigen (Ag). In this example, the polysaccharide multimerization agent was a high MW, branched copolymer of sucrose and epichlorohydrin marketed as FICOLL® marketed by GE Healthcare. However, generic versions or biosimilars (unbranded or other brands) are also suitable for use and hence this moiety is referred to herein as simply Fic.

Tumor Models.

Immunogenic compositions were tested in three different murine tumor models. All three models employed female C57BL/6 mice of 6 to 8 weeks of age, which were purchased from Harlan Laboratories (now Envigo).

EG7-OVA lymphoma model. The EG7-OVA cell line was obtained from ATCC® (American Type Culture Collection, Manassas, Va.). EG7-OVA (Catalog No. CRL-2113™) is a derivative of the murine lymphoma cell line EL4, which was modified to express the model antigen, chicken ovalbumin (OVA) (see, Moore et al., Cell, 54:777-785, 1988). About 1×10⁶ EG7-OVA cells were injected subcutaneously (SC) in the flank of mice in 100 μl PBS to initiate tumor formation. Once tumors had reached a size of 10 to 20 mm³, D61-01-Fic (adjuvant alone) or D61-01-Fic-Ag (vaccine) nanoparticles were administered.

B16-F10 and B16-OVA melanoma models. The B16-F10 cell line was obtained from ATCC® (American Type Culture Collection, Manassas, Va.). B16-F10 (Catalog No. CRL-6475™) is a murine melanoma cell line (Fidler, Cancer Res, 35:218-224, 1975). The B16-OVA cell line is a derivative of B16-F10, which was modified to express OVA. For B16-OVA and B16-F10, about 1×10⁵ cells were injected SC in the flank of mice in 100 μl PBS to initiate tumor formation. Once tumors had reached a size of 4-7 mm in diameter, D61-01-Fic (adjuvant alone) or D61-01-Fic covalently linked to Ag (vaccine) nanoparticles were administered.

Immunogenic Compositions and Treatment Regimens.

Three different types of D61-01-Fic-Ag (vaccine) nanoparticles were tested. D61-01 is a linear chimeric compound having three nucleic acid moieties and two non-nucleic acid moieties as 5′-TCGGCGC-3′-HEG-5′-AACGTTC-3′-HEG-5′-TCGGCGC-3′ (SEQ ID NO:5). D61-01-Fic-OVA particles comprise ovalbumin protein. D61-01-Fic-OVApep particles comprises the ovalbumin polypeptide: CSGLEQLESIINFEKLTEWTSSNVMEERKIKV (SEQ ID NO:11). D61-01-Fic-Triple particles comprise three class I restricted melanoma epitopes (Trp1, Trp2 and gp100) and an artificial PAn class II DR restricted Epitope (PADRE) as a fusion polypeptide: VGALEGPRNQDWLAKXVAAWTLKAAATAYRYHLLSSVYDFFVWLSC in which X is L-cyclohexylalanine (SEQ ID NO:14). Immunogenic compositions were administered by intratumoral injection (IT) or by extratumoral injection, which in this example involved subcutaneous (SC) injection. D61-01-Fic-OVA particles were delivered as a dose containing 50 μg D61-01 and 39 μg OVA in a volume of 150 μl. D61-01-Fic-OVApep particles were delivered as a dose containing 50 μg D61-01 and 56 μg OVApep in a volume of 150 μl. D61-01-Fic-Triple particles were delivered as a dose containing 55 μg D61-01 and 50 μg Triple in a volume of 150 μl. D61-01-Fic particles were delivered as a dose containing 50 μg D61-01 in a volume of 150 μl.

Measurement of Tumor Growth.

Tumor size was determined by microcaliper measurement of three dimensions (length; L, width; W and depth; D) and volume calculated using the following formula: (L×W×D/2).

Results.

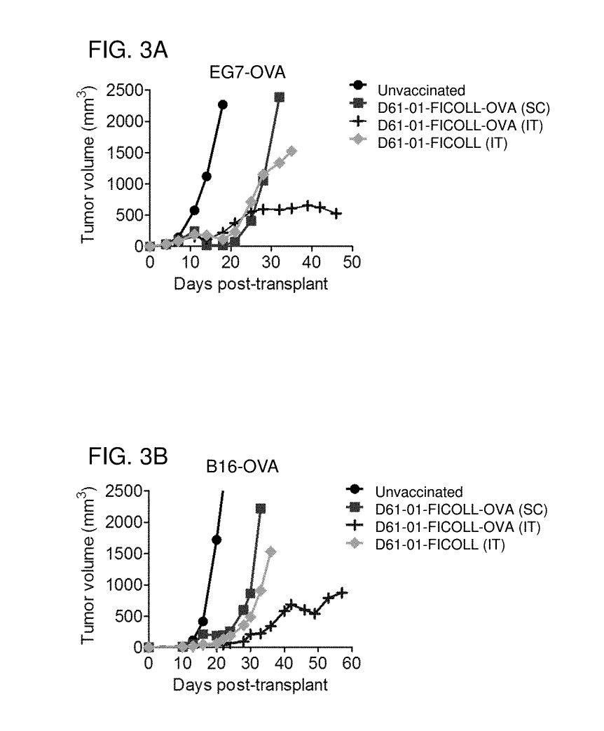

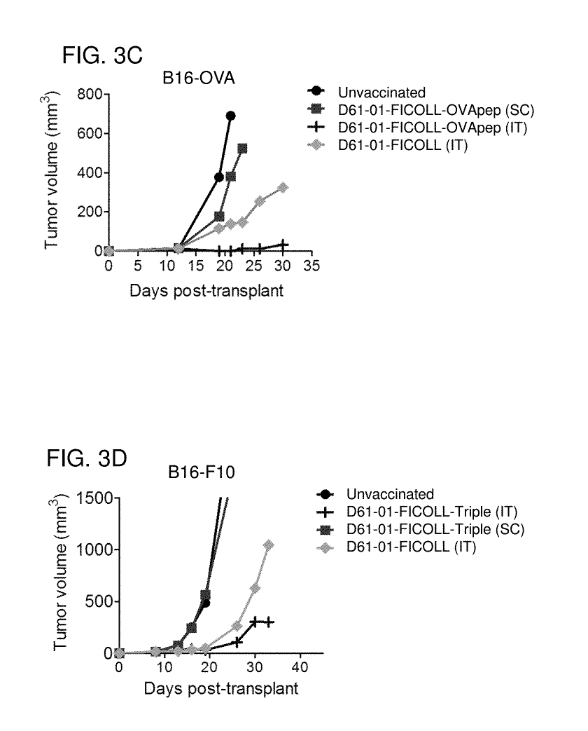

The results of FIG. 3A-D demonstrate that administration of immunogenic compositions by the intratumoral route elicited a superior anti-tumor response when compared to extratumoral administration via subcutaneous injection into a site distant from a tumor. Additionally, intratumoral administration of immunogenic compositions comprising a tumor antigen covalently attached to D61-01-Fic particles elicited a superior anti-tumor response when compared to intratumoral administration of D61-01-Fic particles in the absence of the tumor antigen (adjuvant alone). The efficacy of intratumoral vaccination was not influenced by tumor-type as growth inhibition was observed in tumor models that differed in their tissue of origin (EG7 lymphoma and B16 melanoma). Indeed, it is widely known that the B16 melanoma is a poorly immunogenic tumor (Celik et al., Cancer Res, 43:3507-3510, 1983; Ashman, Immunol Cell Biol, 65:271-77, 1987; and Dezfouli et al., Immunol Cell Biol, 81:459-71, 2003), which makes it an ideal model to assess therapeutic outcomes. In summary, as demonstrated by the exemplary D61-01-Fic platform, a high molecular weight polysaccharide (10 to 1000 kDa) can be used to effectively deliver whole protein antigen (D61-01-Fic-OVA) or polypeptide antigens (D61-01-Fic-OVApep or D61-01-Fic-Triple) with different physiochemical properties to a mammalian subject.

### Example B2: Effect on Tumor Size of Intratumoral Administration of Immunogenic Compositions Comprising CpG and Tumor Antigen Conjugated to the Same or Different Particles

This example describes the control of tumor growth by immunogenic compositions comprising a TLR9 agonist (CpG) and a tumor antigen (Ag) covalently linked to the same or to different polysaccharide molecules. In this example, the polysaccharide multimerization was a high MW, branched copolymer of sucrose and epichlorohydrin marketed as FICOLL® marketed by GE Healthcare. However, generic versions or biosimilars (unbranded or other brands) are also suitable for use and hence this moiety is referred to herein as simply Fic.

Tumor Model.

Immunogenic compositions were tested in the EG7-OVA lymphoma model described in Example B1.

Immunogenic Compositions and Treatment Regimens.

D61-01-Fic-OVApep and D61-01-Fic particles comprise a linear chimeric compound having three nucleic acid moieties and two non-nucleic acid moieties as 5′-TCGGCGC-3′-HEG-5′-AACGTTC-3′-HEG-5′-TCGGCGC-3′ (SEQ ID NO:5). D61-01-Fic-OVApep particles and Fic-OVApep particles comprise the ovalbumin polypeptide:

(SEQ ID NO: 11)

CSGLEQLESIINFEKLTEWTSSNVMEERKIKV.

D61-01-Fic-OVApep particles or a combination of D61-01-Fic and Fic-OVApep particles were delivered intratumoral (IT) injection as a dose containing 50 μg D61-01 and 39 μg OVApep in a volume of 150 μl.

Measurement of Tumor Growth.

Tumor size was determined by microcaliper measurement of three dimensions (length; L, width; W and depth; D) and volume calculated using the following formula: (L×W×D/2).

Results.

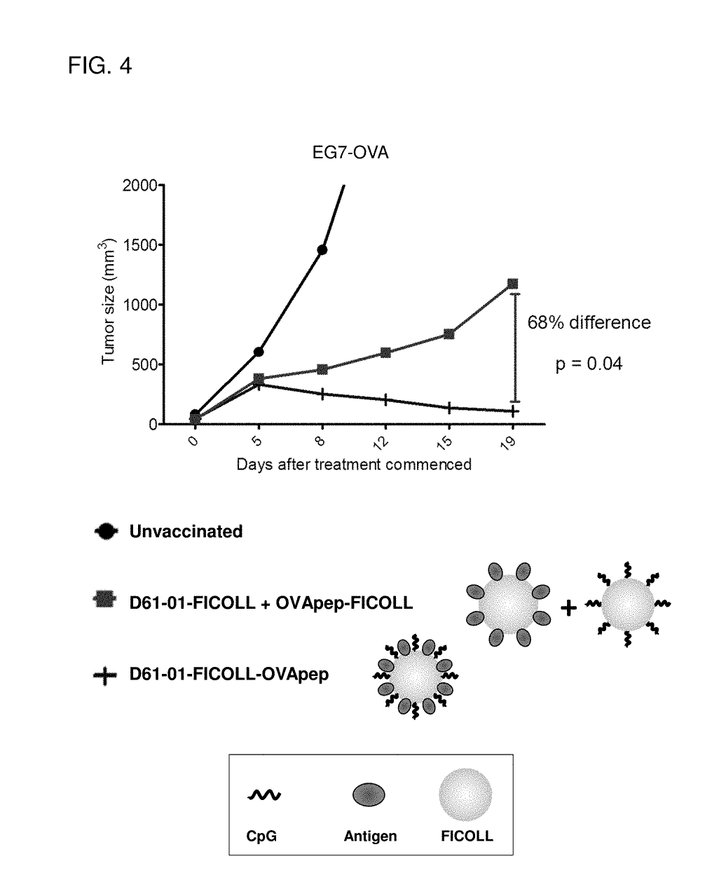

FIG. 4 demonstrates that maximal therapeutic efficacy requires covalent linkage of CpG and tumor antigen to the same particle. Specifically, the intratumoral administration of D61-01 and OVApep covalently attached to the same Fic nanoparticle resulted in a 68% reduction in tumor growth when compared to administration of an immunogenic composition in which D61-01 and OVApep are conjugated to different Fic nanoparticles (568 vs 181 mm³; p=0.04). Statistical significance was calculated using unpaired Student's t-test and GraphPad Prism software, with a p value of less than 0.05 considered to be significant.

### Example B3: Effect of Intratumoral Administration of Immunogenic Compositions on Local Tumor Antigen-Specific Immune Responses

This example describes the elicitation of tumor antigen-specific cellular T cell responses by administration of immunogenic compositions comprising particles comprising a TLR9 agonist (CpG) associated with a polysaccharide multimerization agent alone or in further association with a tumor antigen (Ag). In this example, the polysaccharide multimerization agent was a high MW, branched copolymer of sucrose and epichlorohydrin marketed as FICOLL® marketed by GE Healthcare. However, generic versions or biosimilars (unbranded or other brands) are also suitable for use and hence this moiety is referred to herein as simply Fic.

Tumor Models.

Immunogenic compositions were tested in the EG7-OVA lymphoma and B16-OVA melanoma models described in Example B1.

Immunogenic Compositions and Treatment Regimens.

D61-01 is a linear chimeric compound having three nucleic acid moieties and two non-nucleic acid moieties as

(SEQ ID NO: 5)

5′-TCGGCGC-3′-HEG-5′-AACGTTC-3′-HEG-5′-TCGGCGC-3.′

D61-01-Fic-OVApep particles comprises the ovalbumin polypeptide:

(SEQ ID NO: 11)

CSGLEQLESIINFEKLTEWTSSNVMEERKIKV.

D61-01-Fic-Triple particles comprise three epitopes as a fusion polypeptide: VGALEGPRNQDWLAKXVAAWTLKAAATAYRYHLLSSVYDFFVWLSC in which X is L-cyclohexylalanine (SEQ ID NO:14). Immunogenic compositions were administered by intratumoral injection (IT) or by extratumoral injection, which in this example involved subcutaneous (SC) injection. D61-01-Fic-OVApep particles were delivered as a dose containing 50 μg D61-01 and 56 μg OVApep in a volume of 150 μl. D61-01-Fic-Triple particles were delivered as a dose containing 55 μg D61-01 and 50 μg Triple in a volume of 150 μl. D61-01-Fic particles were delivered as a dose containing 50 μg D61-01 in a volume of 150 μl. Mice bearing established EG7 tumors or B16-OVA tumors received injections on days 0, 7 and 10.

Tumor Processing for Extraction of Infiltrating Leukocytes.

Three days after the last injection, tumors were collected. Tumors were cut into small pieces using a scalpel blade and transferred to a gentleMACS™ tube (Miltenyi Biotec, Auburn, Calif.) containing 10 mL of RPMI-1640 cell culture medium with 5% fetal calf serum (FCS). One hundred microliters of a 100× tumor digestion enzyme mix containing 50 mg/mL collagenase 4 (Sigma-Aldrich C5138-100MG collagenase from Clostridium histolyticum) and 2 mg/mL DNase I (Sigma-Aldrich DN25-100MG deoxyribonuclease I from bovine pancreas) was added to a gentleMACS™ tube (Miltenyi Biotec) containing the tumor fragments. Tumors were dissociated into single cell suspensions using the m_LDK_1 program on the gentleMACS™ Octo dissociator (Miltenyi Biotec) with a 37° C. heating attachment. Samples were filtered through a 70 μm filter. Samples were centrifuged at 1400 rpm for 7 minutes at room temperature and re-suspended in 1 to 5 volumes of 5% FCS in RPMI depending on the size of the tumors.

Isolation of Tumor Infiltrating Leukocytes (TIL).

LYMPHOLYTE® Mammal Cell Separation Media (CEDARLANE® Corporation, Burlington, N.C.) was used to separate tumor infiltrating leukocytes (TIL) from tumor cells. The cell suspension obtained from the tumors was brought to a total volume of 7, 14 or 21 mL by addition of 5% FCS/RPMI. This was determined by the size of the pellet and the requirement to divide the cell suspension into multiple tubes to ensure adequate separation of TIL and tumor cells. A 15 mL conical tube was first filled with 7 mL LYMPHOLYTE®-Mammal Cell Separation Media (Catalog No. CL5120), followed by careful top layering with 7 mL of the cell suspension. Cells were centrifuged at 800×g for 20 min at room temperature (RT) without braking. The layer that forms at the interface between the separation media and the cell culture media was transferred into a 50 mL tube, which was filled with 5% FCS/RPMI medium to a total volume of 50 mL. Cells were pelleted at 1800 rpm for 7 min at RT, under maximum acceleration and maximum braking. The media was aspirated and the TIL-containing pellet re-suspended in 1 mL of 5% FCS in RPMI media.

Generation of Bone-Marrow-Derived Dendritic Cells (BMDCs).

Bone marrow was harvested and pooled from the femurs of three C57BL/6 mice by flushing with purification buffer (0.5% BSA, 2 mM EDTA/PBS). Red blood cells were lysed using 5 mL of red cell lysing buffer (Sigma Catalog No. R7757) for 5 min at RT and the reaction neutralized by the addition of 10 mL purification buffer. Samples were centrifuged for 5 min at 1500 rpm and resuspended in progenitor medium μ (RPMI, 10% FCS, 50 U/mL penicillin, 50 μg/mL streptomycin, 2 mM L-glutamine, 10 mM HEPES, 1 mM sodium pyruvate and 55 μM 2-β-mercaptoethanol) supplemented with 20 ng/mL GM-CSF (Peprotech Catalog No. 315-03) and 10 ng/mL IL4 (Peprotech Catalog No. 214-14) to a final concentration of 1×10⁶ cells/mL. 10 mL of cell suspension was added per non-tissue cultured treated petri dish (BD Falcon Catalog No. 351029) and plates were incubated at 5% CO₂ and 37° C. On day 3 of culture, the cytokines were replenished in each dish by removal of 5 mL of medium and the addition of 5 mL of fresh progenitor medium containing 40 ng/mL GM-CSF and 20 ng/mL IL4. Cultures were incubated for a further 3 days prior to use.

Peptide-Pulsing of BMDCs.

CD11c+ dendritic cells (DCs) were enriched from bone marrow dendritic cell (BMDC) cultures by incubation with pan-DC microbeads (Miltenyi Biotec, Catalog No. 130-100-875) and magnetic separation according to the manufacturer's instructions. Once isolated, BMDC were resuspended at a concentration of 10×10⁶ cells/mL in progenitor medium. A 10 μg/mL final concentration of the OVA class I restricted peptide (SIINFEKL set forth as SEQ ID NO:12) was added and the cells were placed in a 5% CO₂ 37° C. incubator for 30 min. The peptide-pulsed DCs were washed twice with 10 mL progenitor medium and resuspended in 1 mL progenitor medium for subsequent assays.

In Vitro Stimulation of Tumor Infiltrating Leukocytes with Peptide Pulsed Dendritic Cells.

Approximately 250,000 TIL isolated using LYMPHOLYTE® Mammal Cell Separation Media (CEDARLANE® Corporation, Burlington, N.C.) were added per well of a 96-well U-bottomed plate (Corning Costar Catalog No. 3799) and centrifuged at 1600 rpm for 5 min at RT. Peptide-pulsed DCs were resuspended in progenitor medium containing 3 μg/mL brefeldin A (BFA) at a concentration of 400,000 cells/mL. Then, 200 μL of the DC cell suspension was added to the each well containing pelleted TILs and the cells were resuspended by pipetting up and down. TIL-DC co-cultures were centrifuged at 1600 rpm for 1 min to ensure cell to cell contact occurred and incubated for 4 hours at 5% CO₂ 37° C. Samples were analyzed for cytokine production by intracellular staining and flow cytometry.

Intracellular and Cell Surface Staining for Flow Cytometry.

All reagents were kept at 4° C. and all washes performed by addition of 200 μL of FACS buffer (PBS, 10% FCS, 0.1% sodium azide), followed by pipetting the plated cell suspensions up and down, and centrifugation at 1600 rpm for 5 min at 4° C. After 4 hours of incubation, TIL-DC co-cultures were pelleted and resuspended in 45 μL of staining buffer containing 5 μL of the H-2K^{b}/SIINFEKL-APC (Immudex Catalog No. JD2163) or H-2K^{b}/TAYRYHLL-APC (Immudex Catalog No. JD4138) dextramer. SIINFEKL is an OVA class I peptide (SEQ ID NO:12) and TAYRYHLL is a Trp1 class I peptide (SEQ ID NO:15). The staining buffer was a 1:1 mixture of BD Horizon BRILLIANT™ Violet staining buffer (Becton, Dickinson and Co., Franklin Lakes, N.J.) and FACS buffer supplemented with 2 μL/100 μL of Fc block. Samples were incubated for 10 min at 4° C. prior to the addition of 50 μL of staining buffer containing antibodies for cell surface markers. Samples were incubated for a further 20 min at 4° C., washed three times and resuspended in 200 μL of fixation buffer (FACS buffer containing 0.5% paraformaldehyde). Samples were fixed overnight at 4° C. and protected from light.

For intracellular cytokine staining, samples stored overnight at 4° C. were fixed and permeabilized using the BD PHARMINGEN™ Transcription Factor Buffer set (Catalog No. 562725) according to the manufacturer's instructions. Briefly, cells were pelleted and resuspended in 100 μL of 1× Fix/Perm buffer and incubated for 40 min at 4° C. Samples were washed twice with 200 μL 1× Perm/Wash buffer and resuspended in 100 μL of 1× Penn/Wash buffer containing 2.5 μL anti-mouse IFN-γ-PE (BD Biosciences Catalog No. 554412), and 1.5 μL anti-mouse TNF-α-APC-Cy7 (BD Biosciences Catalog No. 560658). Following an additional 40 min incubation at 4° C., samples were washed twice with 200 μL 1× Perm/Wash buffer and resuspended in 300 μL of FACS buffer for analysis. The data was acquired immediately using a LSRII flow cytometer from BD Biosciences (San Jose, Calif.).

Lymph Node Processing for IFN-γ Measurement by ELISA.

Three days after the last injection tumor draining lymph nodes were collected. Single-cell suspensions were generated by passage of lymph nodes in 5 mL 5% FCS/RPMI through a 70 μM filter and homogenization using a 3 mL syringe plunger. Any tissue left on the filter was harvested and added to the tube containing the homogenized lymph nodes. Tissue dissociation enzyme was added and the samples were incubated at 37° C. for 20 min with occasional mixing by inversion of the tube. The reaction was neutralized by the addition of 10 mL of 5% FCS/RPMI. The samples were centrifuged and resuspended in 2 mL of progenitor medium for subsequent assays.

About 500,000 lymph node cells were added to each well of a 96-well U-bottom plate in 100 μL of progenitor medium. Serial dilutions of a 2× concentration of peptide were made and 100 μL was added to each well. IFN-γ was measured in tissue culture supernatants using a commercially available ELISA kit (R & D Systems Catalog No. DY485) according to the manufacturer's instructions. The assay was considered valid if the optical density of the sample fell within the linear range of the standard curve. Values were calculated by subtraction of the background concentration levels, which were determined with reference to controls incubated in the absence of peptide to determine the level of spontaneous cytokine production.

Results.

Tables B3-1 and B3-2 show the magnitude of antigen-specific cytokine production by TIL of mice treated with D61-01-Fic-OVApep or D61-01-Fic-Triple administered by IT or SC injection. Antigen-specific cytokine production by TIL of control mice, which were either left untreated or treated with D61-01-Fic administered by IT injection (adjuvant alone), is also shown. Data represents mean percentage +/−SEM of antigen-specific TIL that simultaneously produce the cytokines TNF-α and IFN-γ. Statistical significance was calculated using unpaired Student's t-test and GraphPad Prism software. Values less than 0.05 were considered significant.

TABLE B3-1
Percentage of EG7-OVA lymphoma TILs that are poly-functional
antigen-specific (OVA class I peptide-specific) lymphocytes\hat{ }

| Group | TNF-α⁺ IFN-γ⁺ |
|---|---|
| Unvaccinated | 1.17 +/− 1.02 |
| D61-01-Fic-OVApep (SC) | 9.02 +/− 0.59 |
| D61-01-Fic-OVApep (IT) | 14.77 +/− 1.84 |
| D61-01-Fic (IT) | 5.21 +/− 1.53 |

\hat{ }p = 0.04 for D61-01-Fic-OVApep (SC) versus D61-01-Fic-OVApep (IT).

TABLE B3-2
Percentage of B16-OVA melanoma TILs that are poly-functional
antigen-specific (Trp1 class I peptide-specific) lymphocytes\hat{ }

| Group | TNF-α⁺ IFN-γ⁺ |
|---|---|
| Unvaccinated | 0.36 +/− 0.13 |
| D61-01-Fic-Triple (SC) | 7.19 +/− 4.21 |
| D61-01-Fic-Triple (IT) | 25.2 +/− 1.22 |
| D61-01-Fic (IT) | 6.03 +/− 4.58 |

\hat{ }p = 0.01 for D61-01-Fic-Triple (SC) versus D61-01-Fic-Triple (IT).

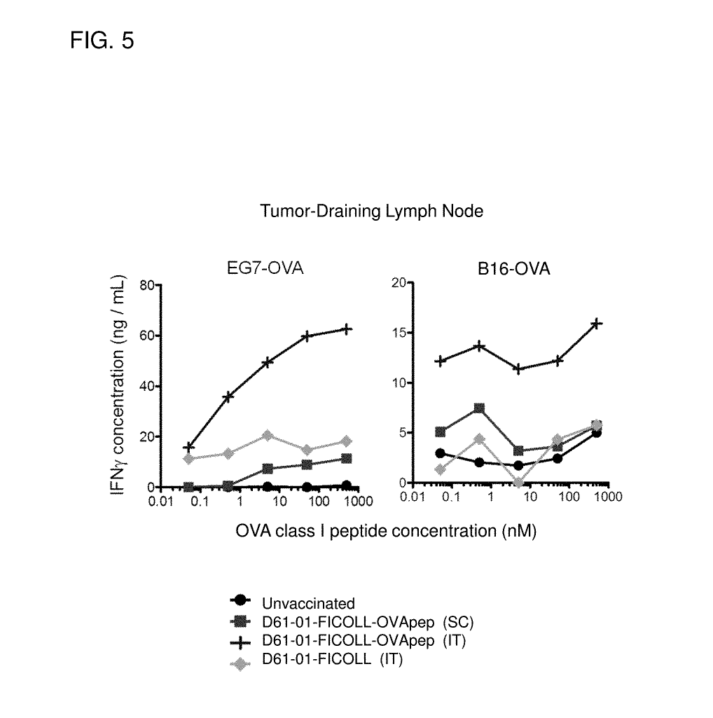

FIG. 5 shows the magnitude of antigen-specific cytokine production by lymphocytes of tumor-draining lymph nodes of mice treated with D61-01-Fic-OVApep administered by IT or SC injection. Antigen-specific cytokine production by lymphocytes of control mice, which were either left untreated or treated with D61-01-Fic administered by IT injection (adjuvant alone), is also shown.

A hallmark of antigen-specific CD8+ T cells with superior cytotoxic function is the simultaneous secretion of multiple cytokines. Acquisition of this phenotype correlates with an enhanced ability to reject tumors (Yuan et al., Proc Natl Acad Sci USA, 105:20410-15, 2008; Aranda et al., Cancer Res, 71:3214, 2011; Mandl et al., J Immunother Cancer, 2:34, 2014; Imai et al., Eur J Immunol, 39:241-53, 2009; and Marshall et al., Cancer Res, 72:581-91, 2012). The secretion of two prototypical Th1 cytokines, TNF-α and IFN-γ was assessed by intracellular FACS analysis following restimulation with cognate peptide. Intratumoral injection of D61-01-Fic-Ag increased the proportion of antigen-specific cells that expressed both cytokines above what was observed for either subcutaneous injection of the D61-01-Fic-Ag or intratumoral injection of D61-01-Fic alone.

Once activated within the tumor-microenvironment, dendritic cells (DCs) travel to tumor draining lymph nodes where they interact with CD8+ T cells to promote their maturation into cytotoxic T lymphocytes (CTLs) capable of tumor destruction. Consequently, the assessment of the tumor antigen-specific recall response of lymph node cells provides a read-out of the ability of the tumor microenvironment to promote the expansion of tumor antigen-specific CTL. Intratumoral injection of D61-01-Fic-Ovapep greatly enhanced the production of IFN-γ from tumor draining lymph node cells in both the EG7-OVA and B16-OVA models. Tumor antigen-specific CD8+ T cells were the prime drivers of this cytokine response as the amount of IFN-γ detected in the culture supernatant was directly proportional to the amount of OVA class I peptide used for stimulation. Taken together, these results demonstrate that intratumoral vaccination promotes the differentiation of antigen-specific CD8+ T cells with increased functionality when compared to subcutaneous vaccination at distant site or intratumoral vaccination in the absence of antigen.

### Example B4: Effect of Intratumoral Administration of Immunogenic Compositions on Systemic Tumor Antigen-Specific Immune Responses

This example describes the elicitation of tumor antigen-specific cellular T cell responses by administration of immunogenic compositions comprising particles comprising a TLR9 agonist (CpG) associated with a polysaccharide multimerization agent alone or in further association with a tumor antigen (Ag). In this example, the polysaccharide multimerization agent was a high MW, branched copolymer of sucrose and epichlorohydrin marketed as FICOLL® marketed by GE Healthcare (Sweden). However, generic versions or biosimilars (unbranded or other brands) are also suitable for use and hence this moiety is referred to herein as simply Fic.

Tumor Models.

Immunogenic compositions were tested in the EG7-OVA lymphoma and B16-OVA melanoma models described in Example B1. For ex vivo analysis of TILs in contralateral tumors, tumor cells were inoculated in both the right and left flanks on the same day. For analysis of the effect of immunogenic compositions on metastases, lung tumors were established by injecting B16-OVA cells by the intravenous route, while subcutaneous tumors were established by injecting B16-OVA cells by the subcutaneous route.

Immunogenic Compositions and Treatment Regimens.

D61-01 is a linear chimeric compound having three nucleic acid moieties and two non-nucleic acid moieties as

(SEQ ID NO: 5)

5′-TCGGCGC-3′-HEG-5′-AACGTTC-3′-HEG-5′-TCGGCGC-3′.

D61-01-Fic-OVApep particles comprises the ovalbumin polypeptide: CSGLEQLESIINFEKLTEWTSSNVMEERKIKV (SEQ ID NO:11). Immunogenic compositions were administered by intratumoral injection (IT) or by extratumoral injection, which in this example involved subcutaneous (SC) injection. D61-01-Fic-OVApep particles were delivered as a dose containing 50 μg D61-01 and 56 μg OVApep in a volume of 150 μl. D61-01-Fic particles were delivered as a dose containing 50 μg D61-01 in a volume of 150 μl. For analysis of splenic lymphocytes, mice bice bearing established EG7 tumors or B16-OVA tumors received injections on days 0, 3 and 7. For analysis of contralateral TILs, mice bearing established B16-OVA tumors on both the right and left flanks received IT injections in either the right tumor or SC injections at a site distant from both the right and left tumors on days 11, 14 and 18 (study one) or on days 10, 13 and 17 (study two). For analysis of the effect of immunogenic compositions on metastasis, mice bearing concommitant subcutaneous and lung tumors received injections of immunogenic compositions on days 8, 12, 15 and 18 after implantation of the subcutaneous tumor.

Spleen Processing and IFN-γ ELISA.

Three days after the last immunization, spleens were collected and splenocytes were isolated. Single-cell suspensions were generated by passage of the spleen in 5 mL 5% FCS/RPMI through a 70 μM filter. The samples were centrifuged and resuspended in 5 mL red cell lysis buffer for 5 min at RT. The reaction was neutralized by the addition of 10 mL of 5% FCS/RPMI and the samples were subsequently centrifuged and resuspended in 2 mL progenitor medium. About 500,000 splenocytes were added to each well of a 96-well U-bottom plate in 100 μL of progenitor medium. Serial dilutions of a 2× concentration of peptide were made and 100 μL added per well. IFN-γ was measured in tissue culture supernatants as described in Example B3.

Isolation of Tumor Infiltrating Leukocytes (TILs).

Tumors were collected and TILs were isolated and stimulated as described in Example B3.

Intracellular and Cell Surface Staining for Flow Cytometry.

TIL samples were resuspended in 45 μL of FACS staining buffer (PBS, 10% FCS, 0.1% sodium azide) containing 5 μL of the H-2Kb/SIINFEKL-APC (Immudex Catalog No. JD2163) dextramer. Samples were incubated for 10 min at 4° C. prior to the addition of 50 μL of staining buffer containing antibodies (1.5 μL per 50 μL) for cell surface markers such as CD107a-PerCP-eFluor710 (eBiosciences Catalog No. 46-1071-82). Samples were incubated for a further 20 min at 4° C., washed three times and resuspended in 200 μL of fixation buffer (FACS buffer containing 0.5% paraformaldehyde). Samples were fixed overnight at 4° C. protected from light. For intracellular staining, samples stored overnight at 4° C. were fixed and permeabilized using the BD PHARMINGEN™ Transcription Factor Buffer set (Catalog No. 562725) according to the manufacturer's instructions. Briefly, cells were pelleted and resuspended in 100 μL of 1× Fix/Perm buffer and incubated for 40 min at 4° C. Samples were washed twice with 200 μL 1× Perm/Wash buffer and resuspended in 100 μL of 1× Perm/Wash buffer containing 2 μL Granzyme B-FITC (Biolegend Catalog No. 515403) or 1.5 μL of Ki67-PE (Biolegend Catalog No. 652404). Following an additional 40 min incubation at 4° C., samples were washed twice with 200 μL 1× Perm/Wash buffer and resuspended in 300 μL of FACS buffer for analysis. The data was acquired immediately using a flow cytometer (LSRII from BD Bioscience). All reagents were kept at 4° C. and all washes performed by addition of 200 μL of buffer, pipetting the plated cell suspensions up and down and centrifugation at 1600 rpm for 5 min at 4° C.

Gene Expression.

RNA was isolated from tumors using an RNEasy® Mini Kit (Qiagen, Valencia, Calif.) according to the manufacturer's instructions. Gene expression analysis was performed on RNA extracted from whole tumors using the nCounter® PanCancer Immune Profiling Panel (NanoString Technologies, Seattle, Wash.). Data analysis was done using nSolver™ analysis software (NanoString Technologies, Seattle, Wash.).

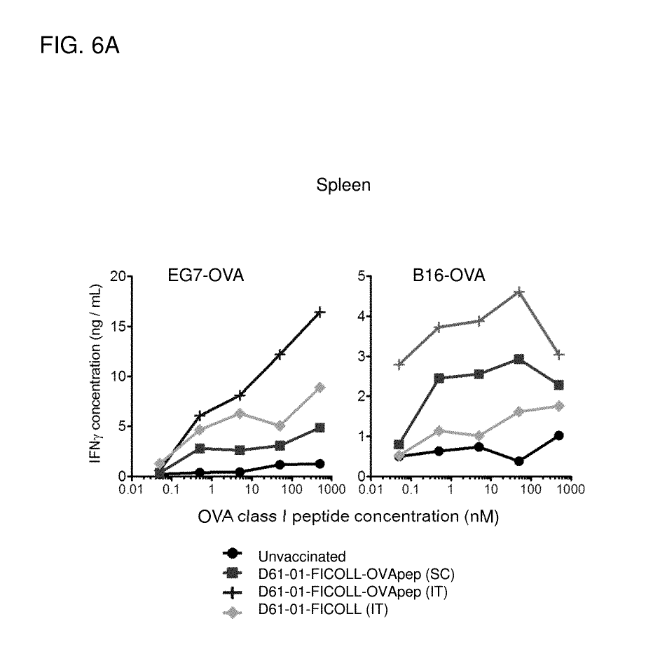

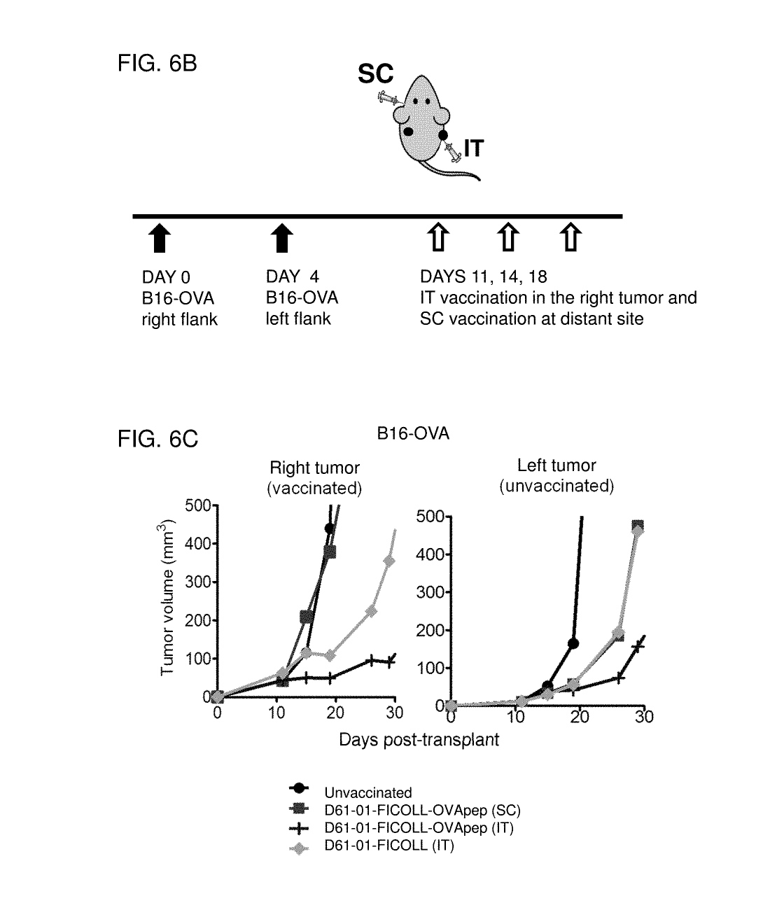

Results. FIG. 6A shows the magnitude of antigen-specific cytokine production by splenocytes of mice treated with D61-01-Fic-OVApep administered by IT or SC injection. Antigen-specific cytokine production by splenocytes of control mice, which were either left untreated or treated with D61-01-Fic administered by IT injection (adjuvant alone), is also shown.

FIG. 6B provides a schematic of the schedule for establishment of bilateral B16-OVA melanoma tumors and subsequent treatment with TLR9 agonist-containing nanoparticles. FIG. 6C show growth curves depicting the change in tumor volume of the contralateral uninjected left tumors and injected right tumors.

Tables B4-1, B4-2 and B4-3 show the characteristics of TIL of contralateral (uninjected) tumors of mice treated with D61-01-Fic-OVApep administered by IT or SC injection. Characteristics of TIL of control mice, which were either left untreated or treated with D61-01-Fic administered by IT injection (adjuvant alone), are also shown. Data represents percentage +/− of 2 or 3 biological replicates per group consisting of a pool of 2-4 independent tumors. Statistical significance was calculated using unpaired Student's t-test and GraphPad Prism software with values less than 0.05 considered to be significant.

TABLE B4-1
Percentage of B16-OVA melanoma TILs from contralateral tumors that
are antigen-specific (OVA class I peptide-specific) lymphocytes\hat{ }

| Group | CD8⁺ SIINFEKL⁺ |
|---|---|
| Unvaccinated | 0.67 +/− 0.09 |
| D61-01-Fic-OVApep (SC) | 8.06 +/− 7.28 |
| D61-01-Fic-OVApep (IT) | 27.7 +/− 2.70 |
| D61-01-Fic (IT) | 15.56 +/− 11.48 |

TABLE B4-2
Percentage of B16-OVA melanoma TILs from contralateral
tumors that are cytotoxic\hat{ }

| Group | GranzB⁺ CD107a⁺ |
|---|---|
| Unvaccinated | 4.65 +/− 1.29 |
| D61-01-Fic-OVApep (SC) | 19.78 +/− 6.57 |
| D61-01-Fic-OVApep (IT) | 39.50 +/− 5.60 |
| D61-01-Fic (IT) | 12.07 +/− 4.50 |

\hat{ }p = 0.01 for D61-01-Fic-OVApep (IT) versus D61-01-Fic (IT).

TABLE B4-3
Percentage of B16-OVA melanoma TILs from contralateral
tumors that are proliferative\hat{ }

| Group | Ki67⁺ |
|---|---|
| Unvaccinated | 5.11 +/− 1.24 |
| D61-01-Fic-OVApep (SC) | 13.43 +/− 1.93 |
| D61-01-Fic-OVApep (IT) | 57.05 +/− 9.65 |
| D61-01-Fic (IT) | 29.4 +/− 1.21 |

\hat{ }p = 0.03 for D61-01-Fic-OVApep (IT) versus D61-01-Fic (IT).

The immunophenotype of TILs from contralateral tumors was determined. Table B4-1 shows that intratumoral injection of CpG-Fic-Ag nanoparticles promotes superior trafficking of tumor antigen-specific CD8+ T cells to the contralateral tumor. Antigen specificity was assessed by measuring binding to an OVA class I peptide. Table B4-2 shows that intratumoral injection of CpG-Fic-Ag nanoparticles enhances cytotoxicity of CD8+ T cells in the contralateral tumor. Cytotoxicity was assessed by staining for expression of the Granzyme B serine protease and the degranulation marker CD107a (LAMP-1). Table B4-3 shows that intratumoral injection of CpG-Fic-Ag nanoparticles enhances the proliferative capacity of CD8+ T cells in the contralateral tumor. Proliferative capacity was assessed by staining for Ki67+ expression.

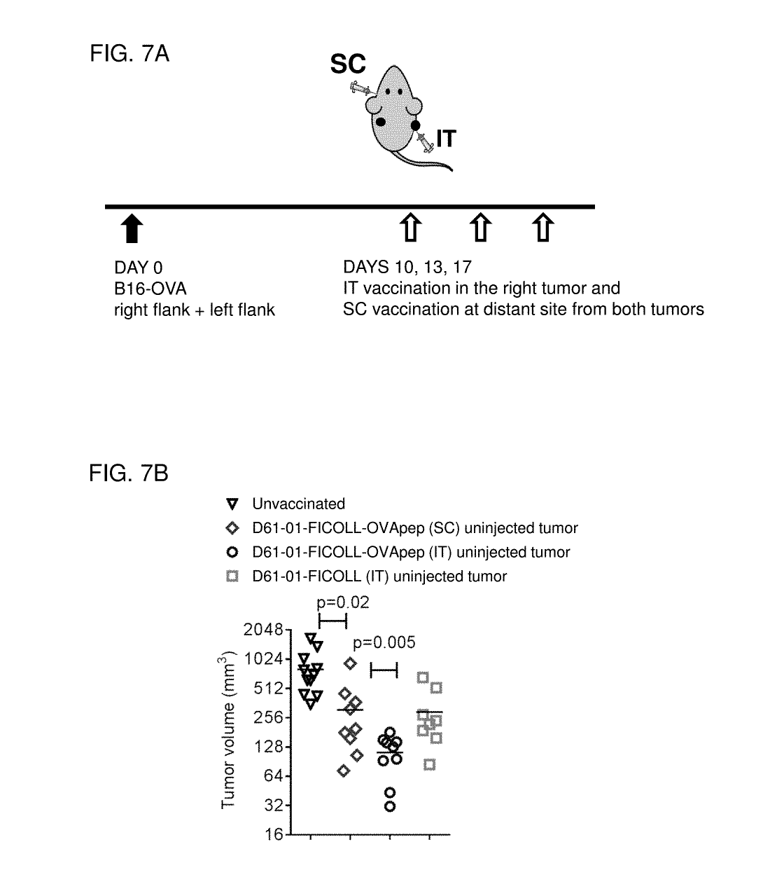

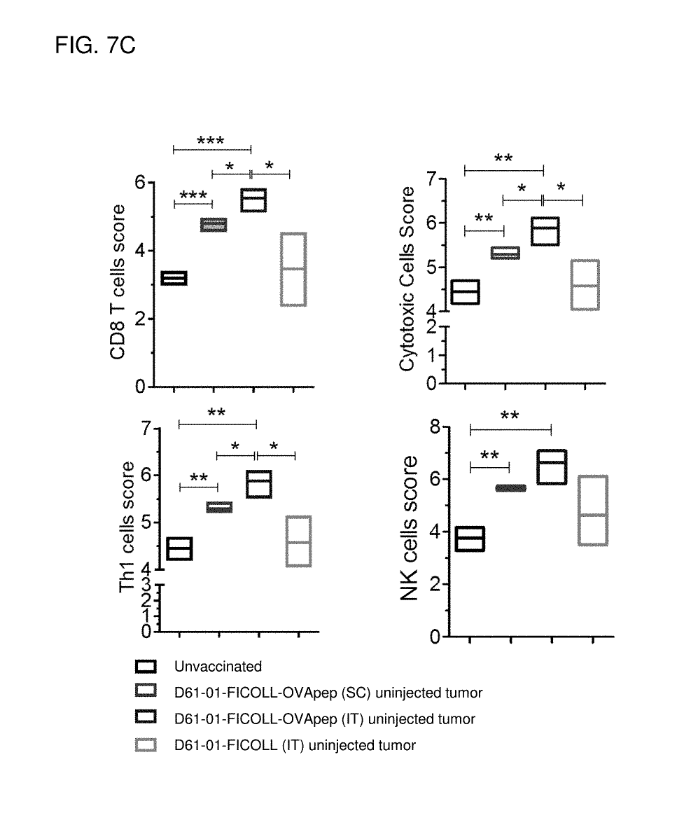

FIG. 7A provides a schematic of the schedule for establishment of bilateral B16-OVA melanoma tumors and subsequent treatment with TLR9 agonist-containing nanoparticles. FIG. 7B shows that administration of an immunogenic composition (D61-01-Fic-OVApep) by the intratumoral route elicited stronger suppression of tumor growth at distant site/uninjected tumors (contralateral) as compared to extratumoral administration via subcutaneous injection. FIG. 7C shows that signatures of CD8+ T cells, cytotoxic cells, Th1 cells and NK cells, are significantly upregulated in uninjected tumors from mice vaccinated IT, as compared to uninjected tumors from mice vaccinated SC, uninjected tumors from mice injected with D61-01-Fic alone by the IT route (IT control), or tumors from unvaccinated mice.

Gene expression analysis was performed on tumor tissue using the nCounter® PanCancer Immune Profiling Panel and nSolver™ analysis software from NanoString Technologies, Inc. (Seattle, Wash.). A directed global significance score was determined, which is a cumulative measure of differential expression in a large set of immune response-related genes. Elevated directed global significance scores, which are indicative of up-regulation of immune responses, are greater than 1.0. Reduced directed global significance scores, which are indicative of down-regulation of immune responses, are less than 1.0.

Table B4-4 shows that the directed global significance score was elevated in uninjected tumors from mice vaccinated IT versus SC with D61-01-Fic-OVApep, or versus uninjected tumors from mice that had received D61-01-Fic alone by the IT route (IT control), relative to tumors from unvaccinated mice. These data show that all immune function signatures of uninjected tumors from mice vaccinated IT are upregulated versus tumors from unvaccinated mice. In addition, all immune function signatures of uninjected tumors from mice vaccinated IT are more intensely upregulated as compared to uninjected tumors from mice vaccinated SC or tumors from mice that had received D61-01-Fic alone by the IT route (IT control).

TABLE B4-4
Relative Directed Global Significance Scores

| | IT vaccine | SC vaccine | IT control |
|---|---|---|---|
| | uninjected | uninjected | uninjected |
| | tumor vs. | tumor vs. | tumor vs. |
| Immune Signature | unvaccinated | unvaccinated | unvaccinated |
| Pathway | tumor | tumor | tumor |
| Adhesion | 4.1 | 3.0 | 1.3 |
| Antigen Processing | 5.2 | 4.3 | 2.0 |
| Apoptosis | 3.7 | 2.7 | 1.4 |
| B-Cell Functions | 3.9 | 2.7 | 0.6 |
| Cell Cycle | 3.4 | 2.2 | 1.3 |
| Complement Pathway | 4.5 | 3.9 | 1.8 |
| Cytokines | 4.0 | 3.1 | 1.4 |
| Dendritic Cell Functions | 4.0 | 3.2 | 1.5 |
| Interferon | 4.6 | 3.5 | 1.7 |
| Interleukins | 4.2 | 3.1 | 1.5 |
| Leukocyte Functions | 3.5 | 2.6 | 0.9 |
| Macrophage Functions | 4.5 | 3.6 | 1.6 |
| MHC class I & II | 5.4 | 4.4 | 2.3 |
| Microglial Functions | 5.4 | 4.0 | 2.2 |
| NK Cell Functions | 3.9 | 3.3 | 1.3 |
| Pathogen Response | 4.1 | 2.9 | 1.2 |
| T-Cell Functions | 4.1 | 3.2 | 1.3 |
| TLR | 4.7 | 3.3 | 2.0 |
| TNF Superfamily | 3.5 | 2.2 | 0.8 |

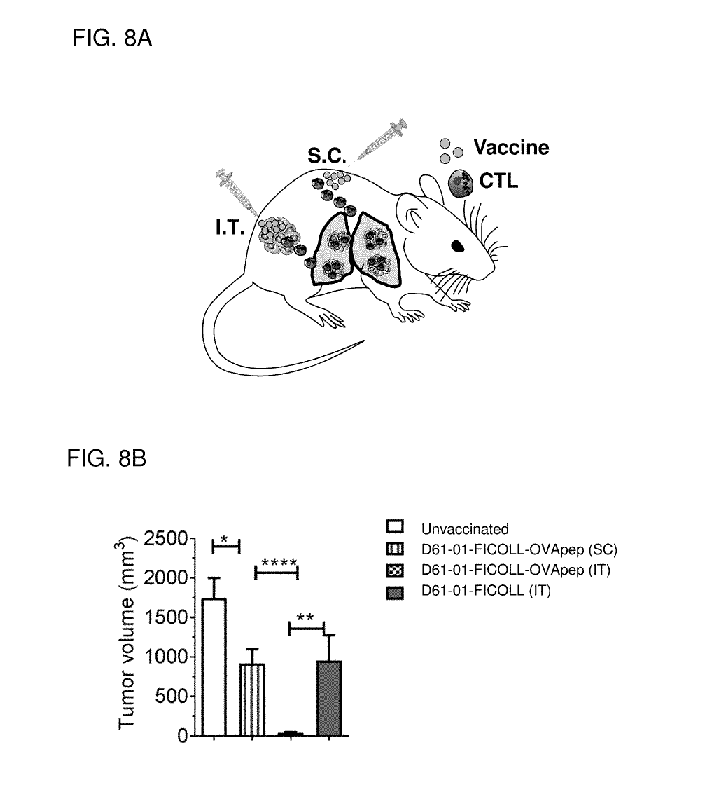

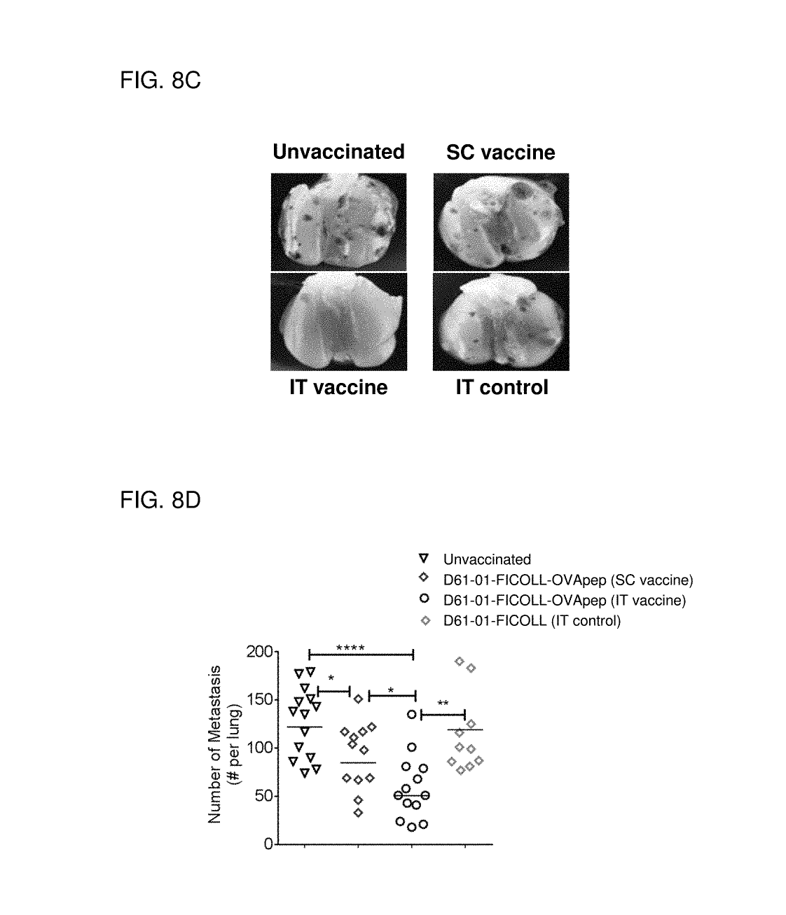

FIG. 8A provides a cartoon showing the establishment of B16-OVA melanoma tumors in both the subcutaneous space and in the lungs of mice. Mice harboring concomitant subcutaneous tumors and lung tumors were vaccinated with D61-01-Fic-OVApep in the subcutaneously growing tumors (IT) or at distant site (SC). D61-01-Fic adjuvant alone given IT was used as control.

FIG. 8B-8D demonstrate that administration of an immunogenic compositions (D61-01-Fic-OVApep) by the intratumoral route elicited a superior anti-tumor response at distant site lung metastasis as compared to extratumoral administration via subcutaneous injection into a site distant from a tumor.

Mortality from cancer is invariably caused by the metastatic spread of primary tumor cells to distant sites in the body. The principal goal of vaccination against cancer is to elicit an immune response capable of not only eradicating the primary tumor but also capable of eliminating disseminated disease (distant site tumors). Tumor-draining lymph nodes receive cells trafficking directly from the tumor, while the spleen provides a reservoir of cells that have trafficked systemically through the peripheral circulation. The development of systemic antigen-specific immunity was confirmed by the dose-dependent induction of IFN-γ from splenocytes isolated from all vaccinated groups in both the EG7 and B16-OVA models. The most robust induction was observed in the intratumoral group vaccinated with CpG-Fic-Ag, demonstrating that intratumoral vaccination induces a superior, tumor antigen-specific, systemic immune response.

Contralateral tumors in mice vaccinated intratumorally with CpG-Fic-Ag grew at a slower rate in comparison to subcutaneously vaccinated mice or mice vaccinated intratumorally with CpG-Fic alone. Accordingly, a greater proportion of antigen-specific CD8+ T cells were found in the contralateral tumors of those mice vaccinated intratumorally with CpG-Fic-Ag (Table B4-1).

Antigen-specific CD8+ T cells exposed to IFN-γ within the Th1-polarized microenvironment generated by intratumoral administration of CpG upregulate expression of the lytic enzyme GranzymeB. The GranzymeB-expressing cells remain poised for recognition of cells presenting a cognate peptide in the context of MHC class I. Once this peptide is recognized, activation-induced degranulation of the CD8+ T cell occurs, which is a necessary precursor of cytolysis. The cell surface exposure of the degranulation marker CD107a is a well-established method to identify this process. The presence of the highly cytotoxic GranzymeB+CD107a+ antigen-specific CD8+ T cells was monitored in contralateral tumors. It was observed that a greater proportion of CD8+ T cells from mice vaccinated intratumorally with CpG-Fic-Ag had acquired this highly cytotoxic phenotype (Table B4-2). In addition, there was an increase in proliferative capacity as a greater proportion of CD8+ T cells expressed the proliferation marker Ki67 (Table B4-3).

Intratumoral administration of CpG-Fic-Ag not only functions to significantly inhibit growth of the primary tumor, but it also induces a systemic immune response that is superior to that elicited by subcutaneous administration of CpG-Fic-Ag or intratumoral administration of CpG-Fic in the absence of Ag. The systemic immune response was characterized by elicitation of CD8+ T cells with enhanced cytotoxicity and proliferative capacity, two characteristics necessary to impart effective control of tumor growth at distant sites.

### Example B5: Effect of Intratumoral Versus Extratumoral Administration of Immunogenic Compositions on Tumor Size and Metastases

This example describes the control of tumor growth by administration of immunogenic compositions comprising particles comprising a TLR9 agonist (CpG) associated with an aluminum salt multimerization agent alone or in further association with a tumor antigen (Ag). In this example, the aluminum salt multimerization agent was an aluminum hydroxide (alum) formulation marketed as ALHYDROGEL® by Brenntag Nordic A/S (Denmark). However, generic versions or biosimilars (unbranded or other brands) are also suitable for use and hence this formulation is referred to herein as Alum.

Tumor Models.

Immunogenic compositions were tested in the EG7-OVA lymphoma and B16-OVA melanoma models described in Example B1. For analysis of the effect of immunogenic compositions on metastases, lung tumors were established by injecting B16-OVA cells by the intravenous route, while subcutaneous tumors were established by injecting B16-OVA cells by the subcutaneous route.

Immunogenic Compositions and Treatment Regimens.

D61-01 is a linear chimeric compound having three nucleic acid moieties and two non-nucleic acid moieties as 5′-TCGGCGC-3′-HEG-5′-AACGTTC-3′-HEG-5′-TCGGCGC-3′ (SEQ ID NO:5). D61-04 is a polynucleotide:

(SEQ ID NO: 6)

5′-TCG AAC GTT CGA ACG TTC GAA CGT TCG AAT-3′.

D61-01-Alum-OVApep and D61-04-Alum-OVApep particles comprise the ovalbumin polypeptide:

(SEQ ID NO: 11)

CSGLEQLESIINFEKLTEWTSSNVMEERKIKV.

D61-01-Alum-Triple particles comprise three epitopes as a fusion polypeptide: VGALEGPRNQDWLAKXVAAWTLKAAATAYRYHLLSSVYDFFVWLSC in which X is L-cyclohexylalanine (SEQ ID NO:14). Immunogenic compositions were administered by intratumoral injection (IT) or by extratumoral injection, which in this example involved subcutaneous (SC) injection. D61-01-Alum-OVApep particles were delivered as a dose containing 50 μg D61-01 and 35 μg OVApep in a volume of 150 μl. D61-01-Alum-Triple particles were delivered as a dose containing 50 μg D61-01 and 35 μg Triple in a volume of 150 μl. D61-01-Alum particles were delivered as a dose containing 50 μg D61-01 in a volume of 150 μl. The D61-04-Alum-OVApep particles were delivered as a dose containing 45 μg D61-01 and 45.6 μg OVApep in a volume of 150 μl. In the D61-01 study, mice bearing established B16-OVA melanoma or EG7-OVA lymphoma tumors received injections on days 8, 11 and 15. In the D61-04 study, mice bearing established B16-OVA melanoma tumors received injections on days 11, 14, 18 and 21 after implantation of the subcutaneous tumor.

Measurement of Tumor Growth.

Tumor size was determined by microcaliper measurement of three dimensions (length; L, width; W and depth; D) and volume calculated using the following formula: (L×W×D/2).

Results.

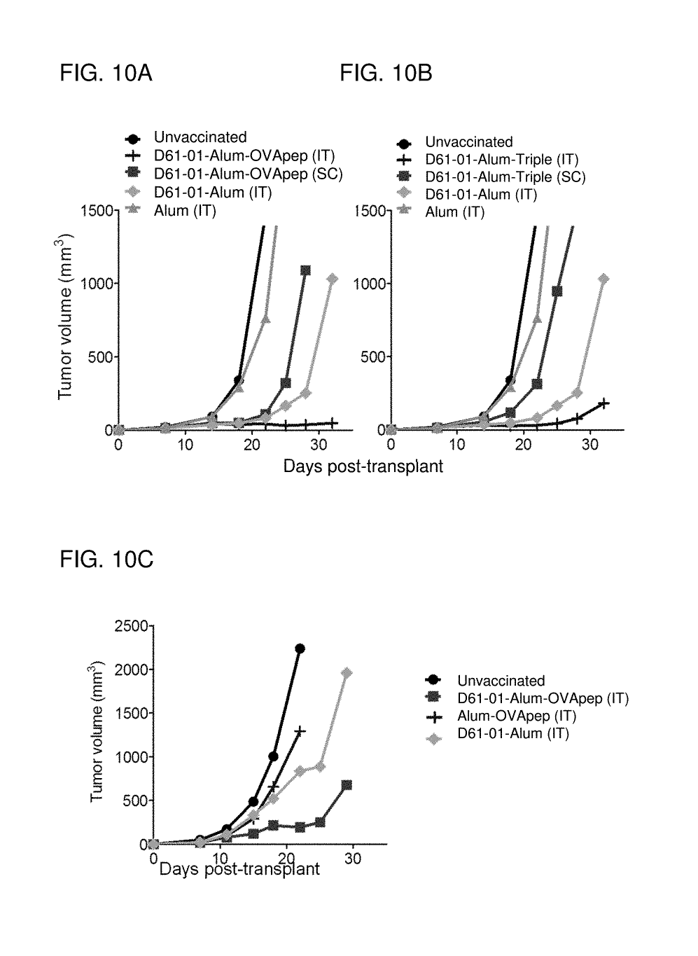

The results of FIG. 10A-C demonstrate that administration of immunogenic compositions by the intratumoral route elicited a superior anti-tumor response when compared to extratumoral administration via subcutaneous injection into a site distant from a tumor.

Advances in high-throughput sequencing technology have catalyzed interest in the identification of patient-specific tumor point mutations that may generate novel epitopes for recognition by CD8+ T cells. The rationale for pursuing these patient-specific neo-epitopes as a source of antigens is that because these epitopes have occurred de novo, they have not been subjected to immune regulation mechanisms that dampen the strength of the CTL response. The personalization of cancer vaccines requires a rapid and efficient method to co-deliver adjuvant and patient-specific neo-antigens to the same cell.

As an alternative to the Fic platform, the use of Alum was explored because preparation of an Alum particulate formulation does not require multi-step chemical conjugation to attach CpG and/or tumor antigens. Similar to what was observed for the CpG-Fic platform, intratumoral administration of D61-01-Alum-OVApep or D61-01-Alum-Triple microparticles generated superior anti-tumor responses when compared to subcutaneous administration or intratumoral administration of D61-01-Alum in the absence of Ag (FIG. 10A-B). Intratumoral administration of Alum alone had no anti-tumor effect, with tumors growing at the same rate as unvaccinated controls. When administered intratumorally, Alum-OVApep or D61-01-Alum failed to achieve the same reduction in tumor growth as the D61-01-Alum-OVApep co-adsorbate (FIG. 10C). This confirmed the requirement for co-absorption of both CpG and antigen to Alum for maximal anti-tumor activity, which replicates what was observed using the Fic platform. Taken together, these results demonstrate that Alum as a particulate formulation can achieve a superior anti-tumor response when co-delivered with CpG adjuvant and antigen intratumorally.

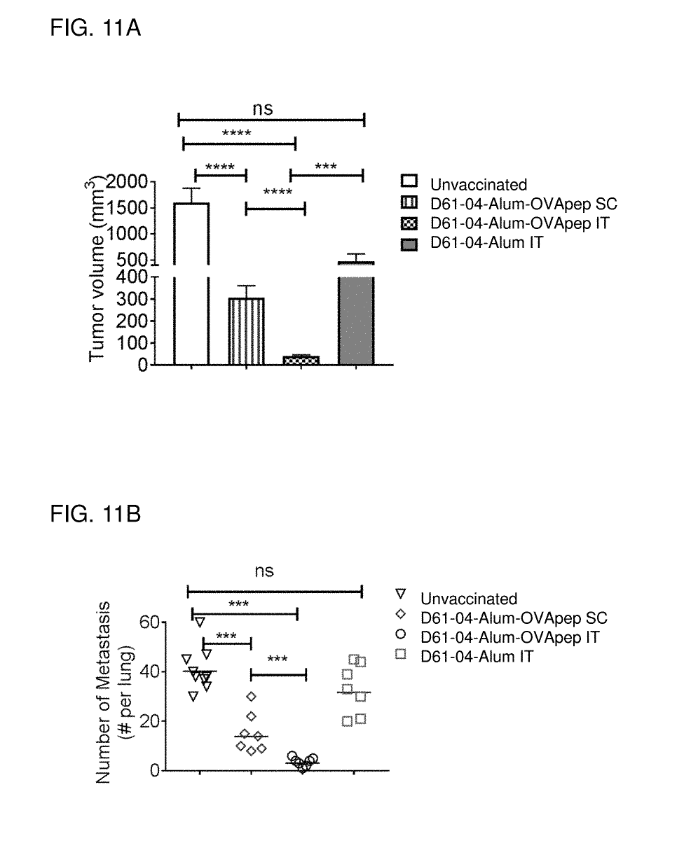

The results of FIG. 11A-B demonstrate that administration of immunogenic compositions (D61-04-Alum-OVApep) by the intratumoral route elicited a superior anti-tumor response in terms of both tumor volume and numbers of distant site lung metastasis as compared to extratumoral administration via subcutaneous injection into a site distant from the tumor. That is, administration of an exemplary immunogenic composition comprising TLR9-alum-tumor antigen particles is efficacious in inducing a systemic immune response capable of reducing tumor volume and eliminating aggressive distant site lung metastases. Moreover, the efficacy of the anti-tumor response is significantly increased when the composition is administered by the intratumoral route.

1. A method of treating cancer in a mammalian subject, the method comprising administering to the subject an effective amount of an immunogenic composition by intratumoral delivery, wherein:
the immunogenic composition comprises a particle comprising a TLR9 agonist and a tumor antigen each associated with a biocompatible multimerization agent;
the multimerization agent has a diameter of about 10 to about 25,000 nanometers and/or a molecular weight of about 10,000 to about 1,000,000 Daltons;
the TLR9 agonist comprises a polynucleotide comprising the sequence 5′-TCGNs-3′ (SEQ ID NO:1), wherein each N is an independently selected nucleoside and s=4 to 47;
the tumor antigen comprises a polypeptide of about 9 to about 1000 amino acids; and
the TLR9 agonist and the tumor antigen are either each associated with the multimerization agent by one or more covalent linkages, or each associated with the multimerization agent by adsorption.

2. The method of claim 1, wherein the multimerization agent comprises an aluminum hydroxide complex having a diameter of about 0.5 to about 25 micrometers, and the TLR9 agonist and the tumor antigen are each associated with the same complex by adsorption.

3. The method of claim 2, wherein the aluminum hydroxide complex has a diameter of about 0.5 to about 5.0 micrometers.

4. The method of claim 1, wherein the multimerization agent comprises a polysaccharide having a diameter of from about 10 to about 1,000 nanometers and/or a molecular weight of about 10,000 to about 1,000,000 Daltons, and the TLR9 agonist and the tumor antigen are each associated with the same molecule of the polysaccharide by one or more covalent linkages.

5. (canceled)

6. The method of claim 4, wherein the polysaccharide is a branched copolymer of sucrose and epichlorohydrin having a molecular weight of about 100,000 to about 700,000 Daltons.

7. The method of claim 1, wherein the TLR9 agonist is a polynucleotide consisting of:
(SEQ ID NO: 2)
5′-(TCG(N_{q}))ᵢN_{w}(X₁X₂CGX₂′X₁′(CG)ₚ)ⱼNᵥ-3′,
wherein each N is an independently selected nucleoside;
p=0 or 1;
q=0, 1, 2, 3, 4 or 5;
v=0 to 41;
w=0, 1 or 2;
i=1, 2, 3 or 4;
j=1, 2, 3 or 4;
X₁ and X₁′ are self-complementary nucleosides; and
X₂ and X₂′ are self-complementary nucleosides; and
wherein the polynucleotide is from 9 to 50 nucleotides in length.

8. The method of claim 1, wherein the TLR9 agonist is a polynucleotide consisting of:
(SEQ ID NO: 3)
5′-TCGN_{q}(X₁X₂CGX₂′X₁′CG)ⱼNᵥ-3′,
wherein each N is an independently selected nucleoside;
q=0, 1, 2, 3, 4, or 5;
v=1 to 39;
j=1, 2, 3 or 4;
X₁ and X₁′ are self-complementary nucleosides; and
X₂ and X₂′ are self-complementary nucleosides;
and wherein the polynucleotide is from 12 to 50 nucleotides in length.

9. The method of claim 1, wherein the TLR9 agonist is a polynucleotide consisting of:
(SEQ ID NO: 4)
5′-TCGN_{q}AACGTTCGAACGTTCGAANᵣ-3′,
wherein each N is an independently selected nucleoside; q=0, 1, 2, 3, 4 or 5; and r=0 to 29.

10. The method of claim 1, wherein the TLR9 agonist is a polynucleotide consisting of a sequence selected from the group consisting of:
(SEQ ID NO: 6)
5′-TCG AAC GTT CGA ACG TTC GAA CGT TCG AAT-3′;
(SEQ ID NO: 7)
5′-TCG TTC GAA CGT TCG AAC GTT CGA A-3′;
(SEQ ID NO: 8)
5′-TCG AAC GTT CGA ACG TTC GAA TTT T-3′;
(SEQ ID NO: 9)
5′-TCG TAA CGT TCG AAC GTT CGA ACG TTA-3′;
and
(SEQ ID NO: 10)
5′-TCG TAA CGT TCG AAC GTT CGA AC-3′.

11. The method of claim 10, wherein the TLR9 agonist is a polynucleotide consisting of 5′-TCG AAC GTT CGA ACG TTC GAA CGT TCG AAT-3′(SEQ ID NO:6).

12. The method of claim 1, wherein the TLR9 agonist is a chimeric compound of the formula Nu1-Sp1-Nu2-Sp2-Nu3,
wherein Nu1, Nu2 and Nu3 are independently selected nucleic acid moieties from 7 to 50 nucleotides in length, and Nu1 consists of the sequence 5′-TCGNs-3′ where s=4 to 47,
wherein Sp1 and Sp2 are the same or different non nucleic acid spacer moieties comprising at least one member of the group consisting of hexaethylene glycol (HEG), triethylene glycol (TEG), propyl, butyl and hexyl, and
wherein Sp1 is covalently linked to Nu1 and Nu2, and Sp2 is covalently linked to Nu2 and Nu3.

13. The method of claim 12, wherein the TLR9 agonist is a chimeric compound comprising three nucleic acid moieties and two hexaethylene glycol (HEG) spacers as
(SEQ ID NO: 5)
5′-TCGGCGC-3′-HEG-5′-AACGTTC-3′-HEG-5′-TCGGCGC-3′
or
(SEQ ID NO: 72)
5′-TCGCCGG-3′-HEG-5′-AACGTTC-3′-HEG-5′-TCGCCGG-3′.

14. The method of claim 1, wherein one or more linkages between nucleotides of the polynucleotide or chimeric compound and/or between the nucleotides and the spacers of the chimeric compound are phosphorothioate ester linkages.

15. The method of claim 14, wherein all of the linkages between nucleotides and between the nucleotides and the spacers are phosphorothioate ester linkages.

16. The method of claim 1, wherein the tumor antigen comprises the amino acid sequence of one of the group consisting of SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:74, and combinations thereof.

17. The method of claim 2, wherein the composition comprises a heterogenous mixture of particles in which the ratio of the TLR9 agonist to the aluminum hydroxide complex and the ratio of the antigen to the aluminum hydroxide complex are each within the range of from about 0.1 to about 5.0 (weight/weight).

18. The method of claim 1, wherein the tumor antigen comprises a polypeptide of about 10 to about 100 amino acids in length.

19. The method of claim 18, wherein the tumor antigen is a fusion protein comprising 2 or more polypeptides, wherein each polypeptide comprises amino acid sequences from different tumor antigens or non-contiguous amino acid sequences from the same tumor antigen.

20. The method of claim 19, wherein the fusion protein comprises a first polypeptide and a second polypeptide, wherein each polypeptide comprises non-contiguous amino acid sequences from the same tumor antigen.

21. The method of claim 18, wherein the tumor antigen comprises a neoantigen encoded by a gene comprising a mutation relative to the gene present in normal cells from the mammalian subject.

22. The method of claim 18, wherein the tumor antigen comprises a viral antigen expressed by the tumor.

23. The method of claim 18, wherein the tumor antigen comprises the amino acid sequence of a human cancer/testis antigen 1 (CTAG1) protein or a fragment thereof.

24. The method of claim 18, wherein the tumor antigen comprises the amino acid sequence of one of the group consisting of SEQ ID NO:52, SEQ ID NO:53, SEQ ID NO:54, SEQ ID NO:55, SEQ ID NO:56, SEQ ID NO:57, SEQ ID NO:58, SEQ ID NO:59, and combinations thereof.

25. The method of claim 1, wherein the mammalian subject is a human.

26. The method of claim 1, wherein intratumoral delivery comprises injection of the immunogenic composition into at least one tumor.

27. The method of claim 26, wherein treating cancer comprises inducing accumulation of tumor antigen-specific T cells in the injected tumor.

28. The method of claim 26, wherein treating cancer comprises eliciting a systemic tumor antigen-specific T cell response.

29. The method of claim 26, wherein treating cancer comprises reducing numbers of CD4+ FoxP3+ regulatory T cells in the injected tumor.

30. The method of claim 26, wherein the subject has one or more uninjected tumors in addition to the injected tumor and treating cancer comprises one or more of the following:
(a) reducing number of uninjected tumors;
(b) reducing volume of uninjected tumors; and
(c) retarding growth of uninjected tumors.

31. The method of claim 26, wherein treating cancer comprises one or more of the following:
(d) increasing survival time of the subject;
(e) reducing volume of the injected tumor; and
(f) retarding growth of the injected tumor.

32. The method of claim 26, wherein the tumor is a sarcoma or a carcinoma.

33. The method of claim 26, wherein the tumor is a lymphoma.

34. The method of claim 1, wherein the cancer is selected from the group consisting of breast cancer, prostate cancer, lung cancer, colorectal cancer, uterine cancer, bladder cancer, melanoma, non-Hodgkin lymphoma, kidney cancer, and thyroid cancer.

35. The method of claim 1, wherein the cancer is a primary cancer of a site selected from the group consisting of oral cavity, digestive system, respiratory system, skin, breast, genital system, urinary system, ocular system, nervous system, endocrine system and lymphoma.

36. The method of claim 1, further comprising administering an effective amount of a second therapeutic agent to the subject.

37. The method of claim 36, wherein the second therapeutic agent comprises a chemotherapeutic agent selected from the group consisting of actinomycin, afatinib, alectinib, asparaginase, azacitidine, azathioprine, bicalutamide, bleomycin, bortezomib, camptothecin, carboplatin, capecitabine, certinib, cisplatin, chlorambucil, crizotinib, cyclophosphamide, cytarabine, daunorubicin, docetaxel, doxifluridine, doxorubicin, erlotinib, epirubicin, epothilone, etoposide, fludarabine, flutamine, fluorouracil, gefitinib, gemcitabine, hydroxyurea, idarubicin, ifosfamide, imatinib, irinotecan, lapatinib, letrozole, mechlorethamine, mercaptopurine, methotrexate, mitomycin, mitoxantrone, octreotide, oxaliplatin, paclitaxel, pemetrexed, raltitrexed, sorafenib, sunitinib, tamoxifen, temozolomide, teniposide, tioguanine, topotecan, valrubicin, vinblastine, vincristine, vindesine, vinorelbine, and combinations thereof.

38. The method of claim 36, wherein the second therapeutic agent is an antagonist of an inhibitory immune checkpoint molecule.

39. The method of claim 38, wherein the inhibitory immune checkpoint molecule is selected from the group consisting of PD-1, PD-L1, PD-L2, CTLA-4 (CD152), LAG-3, TIM-3, TIGIT, IL-10, and TGF-beta.

40. The method of claim 38, wherein the inhibitory immune checkpoint molecule is indoleamine 2,3-dioxygenase (IDO) or tryptophan 2,3-dioxygenase (TDO).

41. The method of claim 36, wherein the second therapeutic agent is an agonist of an immune stimulatory molecule.

42. The method of claim 41, wherein the immune stimulatory molecule is selected from the group consisting of CD27, CD40, OX40 (CD134), GITR, CD137, CD28 and ICOS (CD278).

43. The method of claim 36, wherein the second therapeutic agent comprises an antibody, fragment or derivative thereof.

44. The method of claim 1, further comprising administering radiation therapy.

45. The method of claim 36, wherein the effective amount of the immunogenic composition and the effective amount of the second therapeutic agent together result in a cooperative effect or better against the tumor.

46. The method of claim 36, wherein the effective amount of the immunogenic composition and the effective amount of the second therapeutic agent together result in an additive effect or better against the tumor.

47. The method of claim 36, wherein the effective amount of the immunogenic composition and the effective amount of the second therapeutic agent together result in a synergistic effect against the tumor.

48. The method of claim 1, wherein treating cancer does not result in development of flu-like symptoms of such severity that repeated administration of the immunogenic composition is contraindicated, wherein the flu-like symptoms comprise one or more of the group consisting of fever, headache, chills, myalgia and fatigue.

49. An immunogenic composition comprising a particle comprising a TLR9 agonist and a tumor antigen each associated with a biocompatible multimerization agent, wherein:
the multimerization agent has a diameter of 10 to 10,000 nanometers and/or a molecular weight of about 10,000 to about 1,000,000 Daltons;
the TLR9 agonist comprises a polynucleotide comprising the sequence 5′-TCGNs-3′ (SEQ ID NO:1), wherein each N is an independently selected nucleoside, s=4 to 47;
the tumor antigen comprises a polypeptide of about 9 to about 1000 amino acids; and
the TLR9 agonist and the tumor antigen are either each associated with the multimerization agent by one or more covalent linkages, or each associated with the multimerization agent by adsorption.

50. The composition of claim 49, wherein the multimerization agent is an aluminum hydroxide complex having a diameter of about 500 to about 10,000 micrometers, and the TLR9 agonist and the tumor antigen are each associated with the same complex by adsorption.

51. The composition of claim 50, wherein the aluminum hydroxide complex has a diameter of about 0.5 to about 5.0 micrometers.

52. The composition of claim 49, wherein the multimerization agent is a polysaccharide, and the TLR9 agonist and the tumor antigen are each associated with the same molecule of the polysaccharide by one or more covalent linkages.

53. (canceled)

54. The composition of claim 52, wherein the polysaccharide is a branched copolymer of sucrose and epichlorohydrin having a molecular weight of about 100,000 to about 700,000 Daltons.

55. The composition of claim 54, wherein the particle is a compound of formula (I):
[D-L¹-L²-(PEG)-L³]ₓ-F-[L³-(PEG)-L²-A]ₜ (I),
wherein:
D is the TLR9 agonist;
L¹ is a first linker comprising an alkylthio group;
L² is a second linker comprising a succinimide group;
L³ is a third linker comprising an amide group;
PEG is a polyethylene glycol (e.g., —(OCH₂CH₂)ₙ—, where n is an integer from 2 to 80);
t and x are independently integers from 3 to 200;
A is the tumor antigen; and
F is the polysaccharide, which is connected to L³ via an ether group.

56. The composition of claim 49, wherein the TLR9 agonist is a polynucleotide consisting of
5′-TCGN_{q}AACGTTCGAACGTTCGAANᵣ-3′ (SEQ ID NO:4),
wherein each N is an independently selected nucleoside, q=0, 1, 2, 3, 4 or 5, and r=0 to 29.

57. The composition of claim 56, wherein the TLR9 agonist is a polynucleotide consisting of 5′-TCG AAC GTT CGA ACG TTC GAA CGT TCG AAT-3′ (SEQ ID NO:6).

58. The composition of claim 49, wherein the TLR9 agonist is a chimeric compound of the formula Nu1-Sp1-Nu2-Sp2-Nu3,
wherein Nu1, Nu2 and Nu3 are independently selected nucleic acid moieties from 7 to 50 nucleotides in length, and Nu1 consists of the sequence 5′-TCGNs-3′ where s=4 to 47;
wherein Sp1 and Sp2 are the same or different non nucleic acid spacer moieties comprising at least one member of the group consisting of hexaethylene glycol (HEG), triethylene glycol (TEG), propyl, butyl and hexyl; and
wherein Sp1 is covalently linked to Nu1 and Nu2, and Sp2 is covalently linked to Nu2 and Nu3.

59. The composition of claim 58, wherein the TLR9 agonist is a chimeric compound comprising three nucleic acid moieties and two hexaethylene glycol (HEG) spacers as
(SEQ ID NO: 5)
5′-TCGGCGC-3′-HEG-5′-AACGTTC-3′-HEG-5′-TCGGCGC-3′
or
(SEQ ID NO: 72)
5′-TCGCCGG-3′-HEG-5′-AACGTTC-3′-HEG-5′-TCGCCGG-3′.

60. The composition of claim 49, wherein the tumor antigen comprises a polypeptide of about 10 to about 100 amino acids in length.

61. The composition of claim 60, wherein the tumor antigen is a fusion protein comprising two or more polypeptides, wherein each polypeptide comprises amino acid sequences from different tumor antigens or non-contiguous amino acid sequences from the same tumor antigen.

62. The composition of claim 61, wherein the fusion protein comprises a first polypeptide and a second polypeptide, wherein each polypeptide comprises non-contiguous amino acid sequences from the same tumor antigen.

63. The composition of claim 60, wherein the tumor antigen comprises a neoantigen encoded by a gene comprising a mutation relative to the gene present in normal cells from the mammalian subject.

64. The composition of claim 60, wherein the tumor antigen comprises a viral antigen expressed by the tumor.

65. The composition of claim 60, wherein the tumor antigen comprises the amino acid sequence of a human cancer/testis antigen 1 (CTAG1) protein or a fragment thereof.

66. The composition of claim 60, wherein the tumor antigen comprises the amino acid sequence of one of the group consisting of SEQ ID NO:52, SEQ ID NO:53, SEQ ID NO:54, SEQ ID NO:55, SEQ ID NO:56, SEQ ID NO:57, SEQ ID NO:58, SEQ ID NO:59, and combinations thereof.

67. A method for preparing a compound of formula (I):
[D-L¹-L²-(PEG)-L³]ₓ-F-[L³-(PEG)-L²-A]ₜ (I),
wherein:
D is a TLR9 agonist;
L¹ is a first linker comprising an alkylthio group;
L² is a second linker comprising a succinimide group;
L³ is a third linker comprising an amide group;
PEG is a polyethylene glycol (e.g., —(OCH₂CH₂)ₙ, where n is an integer from 2 to 80);
t and x are independently an integer from 3 to 200;
A is a tumor antigen comprising a polypeptide of about 9 to about 1000 amino acids; and
F is a polysaccharide having a molecular weight of about 10,000 to about 1,000,000 Daltons and is connected to L³ via an ether group,
wherein the TLR9 agonist comprises a polynucleotide comprising the sequence 5′-TCGNs-3′, wherein s=4 to 47 and each N is a nucleoside, and wherein one or more linkages between the nucleotides and between the 3′-terminal nucleotide and L¹ are phosphorothioate ester linkages, and
wherein A is a tumor antigen comprising a polypeptide of about 9 to about 1000 amino acids and comprises at least one thiol group,
the method comprising:
reacting a compound of the formula D-L^{1a}-SH, where D is as defined for formula (I) and L^{1a} is (CH₂)ₘ where m is an integer from 2 to 9, and reacting a compound of the formula A, with a compound of formula (II):
[L^{2a}-(PEG)-L³]_{y}-F (II)
wherein L³, PEG and F are as defined for formula (I);
L^{2a} is 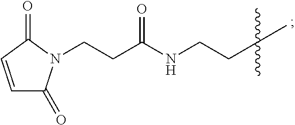 [SMILES: *CCNC(=O)CCN1C(=O)C=CC1=O]
and
y is an integer from 3 to 350; provided that y is no less than the sum of t and x.

68-78. (canceled)

79. A method for preparing a particle comprising a TLR9 agonist and a tumor antigen each associated with a biocompatible multimerization agent by adsorption, wherein:
the multimerization agent is an aluminum hydroxide complex having a diameter of about 0.5 to about 25 micrometers,
the TLR9 agonist comprises a polynucleotide comprising the sequence 5′-TCGNs-3′ (SEQ ID NO:1), wherein s=4 to 47 and each N is a nucleoside, and
the tumor antigen comprises a polypeptide of about 9 to about 1000 amino acids,
the method comprising:
adding the tumor antigen dissolved in an aqueous solution containing about 5% to about 30% isopropanol, and adding the TLR9 agonist, to the aluminum hydroxide complex equilibrated in a buffer,
wherein the buffer is in a pH range of about 6 to about 9 and the buffer is not a phosphate buffer.

80. The method according to claim 79, wherein the aluminum hydroxide complex has a diameter of about 0.5 to about 5.0 micrometers.

81. The method according to claim 80, wherein the buffer is in a pH range of about 7 to about 8.

82. The method according to claim 79, wherein the tumor antigen is dissolved in an aqueous solution containing about 10% to about 20% isopropanol.

83. The method according to claim 79, wherein the TLR9 agonist is dissolved in an acetate buffer having a pH of about 7.

84. The method according to claim 79, wherein the tumor antigen and the TLR9 agonist are adsorbed to the aluminum hydroxide complex at the same time.

85. The method according to claim 79, wherein the tumor antigen is adsorbed to the aluminum hydroxide complex first followed by adsorption of the TLR9 agonist.

86. The method according to claim 79, wherein the TLR9 agonist is adsorbed to the aluminum hydroxide complex first followed by adsorption of the tumor antigen.

87. The method according to claim 79, wherein the TLR9 agonist is a polynucleotide consisting of
5′-TCGN_{q}AACGTTCGAACGTTCGAANᵣ-3′ (SEQ ID NO:4),
wherein each N is an independently selected nucleoside, q=0, 1, 2, 3, 4 or 5, and r=0 to 29.

88. The method according to claim 87, wherein the TLR9 agonist is a polynucleotide consisting of 5′-TCG AAC GTT CGA ACG TTC GAA CGT TCG AAT-3′ (SEQ ID NO:6).

89. The method according to claim 79, wherein the TLR9 agonist is a chimeric compound of the formula Nu1-Sp1-Nu2-Sp2-Nu3,
wherein Nu1, Nu2 and Nu3 are independently selected nucleic acid moieties from 7 to 50 nucleotides in length, and Nu1 consists of the sequence 5′-TCGNs-3′ where s=4 to 47,
wherein Sp1 and Sp2 are the same or different non nucleic acid spacer moieties comprising at least one member of the group consisting of hexaethylene glycol (HEG), triethylene glycol (TEG), propyl, butyl and hexyl, and
wherein Sp1 is covalently linked to Nu1 and Nu2, and Sp2 is covalently linked to Nu2 and Nu3.

90. The method according to claim 89, wherein Nu2 consists of the sequence 5′-AACGTTNm-3′ where m=1 to 44 (SEQ ID NO:73).

91. The method according to claim 90, wherein Nu3 consists of the sequence 5′-AACGTTNm-3′ where m=1 to 44 (SEQ ID NO:73).

92. The method according to claim 89, wherein the TLR9 agonist is
(SEQ ID NO: 5)
5′-TCGGCGC-3′-HEG-5′-AACGTTC-3′-HEG-5′-TCGGCGC-3′,.
or.
(SEQ ID NO: 72)
5′-TCGCCGG-3′-HEG-5′-AACGTTC-3′-HEG-5′-TCGCCGG-3′.

93. The method according to claim 79, wherein the tumor antigen comprises a polypeptide of about 10 to about 100 amino acids in length.

94. The method of claim 93, wherein the tumor antigen is a fusion protein comprising two or more polypeptides, wherein each polypeptide comprises amino acid sequences from different tumor antigens or non-contiguous amino acid sequences from the same tumor antigen.

95. The method of claim 94, wherein the fusion protein comprises a first polypeptide and a second polypeptide, wherein each polypeptide comprises non-contiguous amino acid sequences from the same tumor antigen.

96. The method of claim 93, wherein the tumor antigen comprises a neoantigen encoded by a gene comprising a mutation relative to the gene present in normal cells from the mammalian subject.

97. The method of claim 93, wherein the tumor antigen comprises a viral antigen expressed by the tumor.

98. The method of claim 93, wherein the tumor antigen comprises the amino acid sequence of a human cancer/testis antigen 1 (CTAG1) protein or a fragment thereof.

99. The method of claim 93, wherein the tumor antigen comprises the amino acid sequence of one of the group consisting of SEQ ID NO:52, SEQ ID NO:53, SEQ ID NO:54, SEQ ID NO:55, SEQ ID NO:56, SEQ ID NO:57, SEQ ID NO:58, SEQ ID NO:59, and combinations thereof.

100. A method of preparing a sterile immunogenic composition, comprising the steps of:
(a) dissolving one or more peptide antigens in an aqueous solution comprising an organic solvent to produce an aqueous peptide solution;
(b) contacting the aqueous peptide solution with a slurry comprising an aluminum hydroxide complex to produce particles comprising peptide antigens adsorbed to the aluminum hydroxide complex;
(c) isolating the peptide-aluminum hydroxide particles and reconstitution in a neutral buffer to produce a buffered peptide-aluminum hydroxide particle solution;
(d) autoclaving the buffered peptide-aluminum hydroxide particle solution to produce a sterile particle solution;
(e) dissolving a TLR9 agonist in a neutral buffer to produce a buffered TLR9 agonist solution;
(f) passing the buffered TLR9 agonist solution through an about 0.2 micrometer filter to produce a sterile TLR9 agonist solution; and
(g) contacting the sterile particle solution and the sterile TLR9 agonist solution to produce a sterile immunogenic solution comprising particles comprising the TLR9 agonist and the peptide antigens each adsorbed to the aluminum hydroxide complex; wherein
the one or more peptide antigens are tumor antigens each comprising a polypeptide of about 9 to 2000 amino acids in length,
the aluminum hydroxide complex has a diameter of about 500 to about 5,000 nanometers, and
the TLR9 agonist comprises a CpG-containing polynucleotide of 12 to 50 nucleotides in length.

101. The method of claim 100, wherein the organic solvent is selected from the group consisting of isopropyl alcohol, dimethyl sulfoxide, dimethyformamide, formic acid, ethanol, 2-butanol, acetone, acetic acid, and combinations thereof.

102. The method of claim 100, wherein the tumor antigens each comprise a polypeptide of about 8 to about 60 amino acids in length.

103. The method of claim 100, wherein the neutral buffer is in a pH range of about 6 to about 9 and the buffer is not a phosphate buffer.

104. The method of claim 100, wherein steps (a)-(d) occur before or concurrently with steps (e) and (f).

105. The method of claim 100, wherein the sterile immunogenic composition comprises a heterogeneous mixture of particles in which the ratio of each of the peptide antigens to the aluminum hydroxide complex and the ratio of the TLR9 agonist to the aluminum hydroxide complex are within the range of about 0.1 to about 5.0 (weight/weight).

106. The method of claim 100, wherein the TLR9 agonist comprises the sequence 5′-TCGNs-3′ (SEQ ID NO:1), wherein s=4 to 47 and each N is a nucleoside.

107. The method of claim 106, wherein the TLR9 agonist is a polynucleotide consisting of 5′-TCGN_{q}AACGTTCGAACGTTCGAANᵣ-3′ (SEQ ID NO:4),
wherein each N is an independently selected nucleoside, q=0, 1, 2, 3, 4 or 5, and r=0 to 29.

108. The method of claim 107, wherein the TLR9 agonist is a polynucleotide consisting of 5′-TCG AAC GTT CGA ACG TTC GAA CGT TCG AAT-3′ (SEQ ID NO:6).

109. The method of claim 108, wherein the sterile immunogenic composition comprises a heterogeneous mixture of particles in which the ratio of each of the peptide antigens to the aluminum hydroxide complex is in the range of about 0.6 to 1.2:1.0 (w/w), and the ratio of the TLR9 agonist to the aluminum hydroxide complex is in the range of about 1.7 to 3.4:1.0 (w/w).

110. The method of claim 109, wherein the ratio of each of the peptide antigens to the aluminum hydroxide complex is about 1.2:1.0 (w/w), and the ratio of the TLR9 agonist to the aluminum hydroxide complex is about 3.4:1.0 (w/w).

111. The method of claim 100, wherein the TLR9 agonist is a chimeric compound of the formula Nu1-Sp1-Nu2-Sp2-Nu3,
wherein Nu1, Nu2 and Nu3 are independently selected nucleic acid moieties from 7 to 50 nucleotides in length, and Nu1 consists of the sequence 5′-TCGNs-3′ where s=4 to 47,
wherein Sp1 and Sp2 are the same or different non nucleic acid spacer moieties comprising at least one member of the group consisting of hexaethylene glycol (HEG), triethylene glycol (TEG), propyl, butyl and hexyl, and
wherein Sp1 is covalently linked to Nu1 and Nu2, and Sp2 is covalently linked to Nu2 and Nu3.

112. The method of claim 111, wherein the TLR9 agonist is a chimeric compound comprising three nucleic acid moieties and two hexaethylene glycol (HEG) spacers as
(SEQ ID NO: 5)
5′-TCGGCGC-3′-HEG-5′-AACGTTC-3′-HEG-5′-TCGGCGC-3′
or
(SEQ ID NO: 72)
5′-TCGCCGG-3′-HEG-5′-AACGTTC-3′-HEG-5′-TCGCCGG-3′.

113. The method of claim 36, wherein the second therapeutic agent comprises an epigenetic modulator selected from the group consisting of voronistat, romidepsin, entinostat, abexinostat, elinostat, panobinostat, quisinostat, 4SC-202, resminostat, pracinostat, valproate, and combinations thereof.

114. The composition of claim 49, wherein the tumor antigen comprises the amino acid sequence of one of the group consisting of SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:74, and combinations thereof.

115. The method of claim 79, wherein the tumor antigen comprises the amino acid sequence of one of the group consisting of SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:74, and combinations thereof.


SEQUENCE LISTING (74 sequences)

SEQ ID NO: 1 - DNA, 50 bp, Artificial Sequence
  Synthetic Construct
  misc_feature 4, 5, 6, 7, 8, 9, 10, 11, 12, 13, 14, 15, 16, 17, 18,19, 20, 21, 22, 23, 24, 25, 26, 27, 28, 29, 30, 31, 32, 33,34, 35, 36, 37, 38, 39, 40, 41, 42, 43, 44, 45, 46, 47,48, 49, 50: n = any nucleoside and up to 43 can be present or absent
tcgnnnnnnnnnnnnnnnnnnnnnnnnnnnnnnnnnnnnnnnnnnnnnnn

SEQ ID NO: 2 - DNA, 59 bp, Artificial Sequence
  Synthetic Construct
  misc_feature 4, 5, 6, 7, 8: n = any nucleoside and up to 5 can be present or absent
  misc_feature 1, 2, 3, 4, 5, 6, 7, 8: can be present in repeat of up to 4
  misc_feature 9, 10: n = any nucleoside and up to 2 can be present or absent
  misc_feature 11, 16: n = any nucleoside, and position 11 and 16 are self-complementary
  misc_feature 12, 15: n = any nucleoside, and position 12 and 15 are self-complementary
  misc_feature 17, 18: can be present or absent
  misc_feature 11, 12, 13, 14, 15, 16, 17, 18: can be present in repeat of up to 4
  misc_feature 19, 20, 21, 22, 23, 24, 25, 26, 27, 28, 29, 30, 31,32, 33, 34, 35, 36, 37, 38, 39, 40, 41, 42, 43, 44, 45, 4647, 48, 49, 50, 51, 52, 53, 54, 55, 56, 57, 58, 59: n = any nucleoside and up to 41 can be present or absent
tcgnnnnnnnnncgnncgnnnnnnnnnnnnnnnnnnnnnnnnnnnnnnnnnnnnnnnnn

SEQ ID NO: 3 - DNA, 55 bp, Artificial Sequence
  Synthetic Construct
  misc_feature 4, 5, 6, 7, 8: n = any nucleoside and up to 5 can be present or absent
  misc_feature 9, 14: n = any nucleoside, and position 9 and 14 are self-complementary
  misc_feature 10, 13: n = any nucleoside, and position 10 and 13 are self-complementary
  misc_feature 9, 10, 11, 12, 13, 14, 15, 16: can be present in repeat of up to 4
  misc_feature 17, 18, 19, 20, 21, 22, 23, 24, 25, 26, 27, 28, 29,30, 31, 32, 33, 34, 35, 36, 37, 38, 39, 40, 41, 42,43, 44, 45, 46, 47, 48, 49, 50, 51, 52, 53, 54, 55: n = any nucleoside and up to 38 can be present or absent
tcgnnnnnnncgnncgnnnnnnnnnnnnnnnnnnnnnnnnnnnnnnnnnnnnnnn

SEQ ID NO: 4 - DNA, 55 bp, Artificial Sequence
  Synthetic Construct
  misc_feature 4, 5, 6, 7, 8: n = any nucleoside and up to 5 can be present or absent
  misc_feature 27, 28, 29, 30, 31, 32, 33, 34, 35, 36, 37, 38, 39, 40,41, 42, 43, 44, 45, 46, 47, 48, 49, 50, 51, 52, 53, 54, 55: n = any nucleoside and up to 29 can be present or absent
tcgnnnnnaacgttcgaacgttcgaannnnnnnnnnnnnnnnnnnnnnnnnnnnn

SEQ ID NO: 5 - DNA, 21 bp, Artificial Sequence
  Synthetic Construct
  misc_feature 7, 8: positions 7 and 8 are linked by a hexa-(ethylene glycol) moiety
  misc_feature 14, 15: positions 14 and 15 are linked by a hexa-(ethylene glycol) moiety
tcggcgcaacgttctcggcgc

SEQ ID NO: 6 - DNA, 30 bp, Artificial Sequence
  Synthetic Construct
tcgaacgttcgaacgttcgaacgttcgaat

SEQ ID NO: 7 - DNA, 25 bp, Artificial Sequence
  Synthetic Construct
tcgttcgaacgttcgaacgttcgaa

SEQ ID NO: 8 - DNA, 25 bp, Artificial Sequence
  Synthetic Construct
tcgaacgttcgaacgttcgaatttt

SEQ ID NO: 9 - DNA, 27 bp, Artificial Sequence
  Synthetic Construct
tcgtaacgttcgaacgttcgaacgtta

SEQ ID NO: 10 - DNA, 23 bp, Artificial Sequence
  Synthetic Construct
tcgtaacgttcgaacgttcgaac

SEQ ID NO: 11 - protein, 32 aa, Artificial Sequence
  Synthetic Construct
CSGLEQLESIINFEKLTEWTSSNVMEERKIKV

SEQ ID NO: 12 - protein, 8 aa, Artificial Sequence
  Synthetic Construct
SIINFEKL

SEQ ID NO: 13 - protein, 16 aa, Artificial Sequence
  Synthetic Construct
TEWTSSNVMEERKIKV

SEQ ID NO: 14 - protein, 46 aa, Artificial Sequence
  Synthetic Construct
  VARIANT 1: attached to (polyethylene glycol)24
  VARIANT 16: Xaa = L-cyclohexylalanine
VGALEGPRNQDWLAKXVAAWTLKAAATAYRYHLLSSVYDFFVWLSC

SEQ ID NO: 15 - protein, 8 aa, Artificial Sequence
  Synthetic Construct
TAYRYHLL

SEQ ID NO: 16 - protein, 9 aa, Artificial Sequence
  Synthetic Construct
SVYDFFVWL

SEQ ID NO: 17 - protein, 9 aa, Artificial Sequence
  Synthetic Construct
EGPRNQDWL

SEQ ID NO: 18 - protein, 13 aa, Artificial Sequence
  Synthetic Construct
  VARIANT 3: Xaa = L-cyclohexylalanine
AKXVAAWTLKAAA

SEQ ID NO: 19 - protein, 28 aa, Artificial Sequence
  Synthetic Construct
EVYDFAFRDLQAEPDRAHYNIVTFCCKC

SEQ ID NO: 20 - protein, 10 aa, Artificial Sequence
  Synthetic Construct
EVYDFAFRDL

SEQ ID NO: 21 - protein, 14 aa, Artificial Sequence
  Synthetic Construct
QAEPDRAHYNIVTF

SEQ ID NO: 22 - protein, 42 aa, Artificial Sequence
  Synthetic Construct
PFPAAVILRDALHWNDLAVIPAGVVHNEFVDWENVSPELNST

SEQ ID NO: 23 - protein, 12 aa, Artificial Sequence
  Synthetic Construct
PFPAAVILRDAL

SEQ ID NO: 24 - protein, 15 aa, Artificial Sequence
  Synthetic Construct
HWNDLAVIPAGVVHN

SEQ ID NO: 25 - protein, 15 aa, Artificial Sequence
  Synthetic Construct
EFVDWENVSPELNST

SEQ ID NO: 26 - protein, 34 aa, Artificial Sequence
  Synthetic Construct
VTYHSPSYVYHQFERRAKLVQFIKDRISVVQASC

SEQ ID NO: 27 - protein, 39 aa, Artificial Sequence
  Synthetic Construct
  VARIANT 1: attached to (polyethylene glycol)24
AAVILRDALHWNDLAVIPAGVVHNEFVDWENVSPELNST

SEQ ID NO: 28 - protein, 52 aa, Artificial Sequence
  Synthetic Construct
  VARIANT 42: Xaa = L-cyclohexylalanine
VGALEGPRNQDWLAAYTAYRYHLLAAYSVYDFFVWLAAYALXVAAWTLKAAA

SEQ ID NO: 29 - protein, 24 aa, Artificial Sequence
  Synthetic Construct
  VARIANT 3: Xaa = L-cyclohexylalanine
AKXVAAWTLKAAAAAYTAYRYHLL

SEQ ID NO: 30 - protein, 20 aa, Artificial Sequence
  Synthetic Construct
AVGALEGPRNQDWLGVPRQL

SEQ ID NO: 31 - protein, 19 aa, Artificial Sequence
  Synthetic Construct
QIANCSVYDFFVWLHYYSV

SEQ ID NO: 32 - protein, 28 aa, Artificial Sequence
  Synthetic Construct
EVYNFAFRDLQAEPDRAHYNIVTFCCKC

SEQ ID NO: 33 - protein, 27 aa, Artificial Sequence
  Synthetic Construct
SHCHWNDLAVIPAGVVHNWDFEPRKVS

SEQ ID NO: 34 - protein, 27 aa, Artificial Sequence
  Synthetic Construct
PSKPSFQEFVDWENVSPELNSTDQPFL

SEQ ID NO: 35 - protein, 27 aa, Artificial Sequence
  Synthetic Construct
DSGSPFPAAVILRDALHMARGLKYLHQ

SEQ ID NO: 36 - protein, 37 aa, Artificial Sequence
  Synthetic Construct
VTYHSPSYVYHQFERRAKAAYLVQFIKDRISVVQASC

SEQ ID NO: 37 - protein, 34 aa, Artificial Sequence; identical to SEQ ID NO: 26
  Synthetic Construct
  VARIANT 1: attached to (polyethylene glycol)24

SEQ ID NO: 38 - protein, 11 aa, Artificial Sequence
  Synthetic Construct
SSDYVIPIGTY

SEQ ID NO: 39 - protein, 12 aa, Artificial Sequence
  Synthetic Construct
DAEKSDICTDEY

SEQ ID NO: 40 - protein, 9 aa, Artificial Sequence
  Synthetic Construct
YMDGTMSQV

SEQ ID NO: 41 - protein, 9 aa, Artificial Sequence
  Synthetic Construct
ALLAVGATK

SEQ ID NO: 42 - protein, 9 aa, Artificial Sequence
  Synthetic Construct
IMDQVPFSV

SEQ ID NO: 43 - protein, 30 aa, Artificial Sequence
  Synthetic Construct
VPLAHSSSAFTIMDQVPFSVSVSQLRALDG

SEQ ID NO: 44 - protein, 9 aa, Artificial Sequence
  Synthetic Construct
YLEPGPVTA

SEQ ID NO: 45 - protein, 9 aa, Artificial Sequence
  Synthetic Construct
LIYRRRLMK

SEQ ID NO: 46 - protein, 9 aa, Artificial Sequence
  Synthetic Construct
SLFRAVITK

SEQ ID NO: 47 - protein, 9 aa, Artificial Sequence
  Synthetic Construct
EADPTGHSY

SEQ ID NO: 48 - protein, 9 aa, Artificial Sequence
  Synthetic Construct
EVDPIGHLY

SEQ ID NO: 49 - protein, 9 aa, Artificial Sequence
  Synthetic Construct
GLYDGMEHL

SEQ ID NO: 50 - protein, 30 aa, Artificial Sequence
  Synthetic Construct
VIWEALNMMGLYDGMEHLIYGEPRKLLTQD

SEQ ID NO: 51 - protein, 16 aa, Artificial Sequence
  Synthetic Construct
AQYIKANSKFIGITEL

SEQ ID NO: 52 - protein, 10 aa, Artificial Sequence
  Synthetic Construct
ASGPGGGAPR

SEQ ID NO: 53 - protein, 9 aa, Artificial Sequence
  Synthetic Construct
MPFATPMEA

SEQ ID NO: 54 - protein, 9 aa, Artificial Sequence
  Synthetic Construct
SLLMWITQC

SEQ ID NO: 55 - protein, 30 aa, Artificial Sequence
  Synthetic Construct
GARGPESRLLEFYLAMPFATPMEAELARRS

SEQ ID NO: 56 - protein, 30 aa, Artificial Sequence
  Synthetic Construct
MEAELARRSLAQDAPPLPVPGVLLKEFTVS

SEQ ID NO: 57 - protein, 30 aa, Artificial Sequence
  Synthetic Construct
VLLKEFTVSGNILTIRLTAADHRQLQLSIS

SEQ ID NO: 58 - protein, 32 aa, Artificial Sequence
  Synthetic Construct
HRQLQLSISSCLQQLSLLMWITQCFLPVFLAQ

SEQ ID NO: 59 - protein, 95 aa, Artificial Sequence
  Synthetic Construct
GARGPESRLLEFYLAMPFATPMEAELARRSLAQDAPPLPVPGVLLKEFTVSGNILTIRLTAADHRQLQLSISSCLQQLSLLMWITQCFLPVFLAQ

SEQ ID NO: 60 - protein, 180 aa, Homo sapiens
MQAEGRGTGGSTGDADGPGGPGIPDGPGGNAGGPGEAGATGGRGPRGAGAARASGPGGGAPRGPHGGAASGLNGCCRCGARGPESRLLEFYLAMPFATPMEAELARRSLAQDAPPLPVPGVLLKEFTVSGNILTIRLTAADHRQLQLSISSCLQQLSLLMWITQCFLPVFLAQPPSGQRR

SEQ ID NO: 61 - protein, 309 aa, Homo sapiens
MSLEQRSLHCKPEEALEAQQEALGLVCVQAATSSSSPLVLGTLEEVPTAGSTDPPQSPQGASAFPTTINFTRQRQPSEGSSSREEEGPSTSCILESLFRAVITKKVADLVGFLLLKYRAREPVTKAEMLESVIKNYKHCFPEIFGKASESLQLVFGIDVKEADPTGHSYVLVTCLGLSYDGLLGDNQIMPKTGFLIIVLVMIAMEGGHAPEEEIWEELSVMEVYDGREHSAYGEPRKLLTQDLVQEKYLEYRQVPDSDPARYEFLWGPRALAETSYVKVLEYVIKVSARVRFFFPSLREAALREEEEGV

SEQ ID NO: 62 - protein, 314 aa, Homo sapiens
MPLEQRSQHCKPEEGLEARGEALGLVGAQAPATEEQQTASSSSTLVEVTLGEVPAADSPSPPHSPQGASSFSTTINYTLWRQSDEGSSNQEEEGPRMFPDLESEFQAAISRKMVELVHFLLLKYRAREPVTKAEMLESVLRNCQDFFPVIFSKASEYLQLVFGIEVVEVVPISHLYILVTCLGLSYDGLLGDNQVMPKTGLLIIVLAIIAIEGDCAPEEKIWEELSMLEVFEGREDSVFAHPRKLLMQDLVQENYLEYRQVPGSDPACYEFLWGPRALIETSYVKVLHHTLKIGGEPHISYPPLHERALREGEE

SEQ ID NO: 63 - protein, 314 aa, Homo sapiens
MPLEQRSQHCKPEEGLEARGEALGLVGAQAPATEEQEAASSSSTLVEVTLGEVPAAESPDPPQSPQGASSLPTTMNYPLWSQSYEDSSNQEEEGPSTFPDLESEFQAALSRKVAELVHFLLLKYRAREPVTKAEMLGSVVGNWQYFFPVIFSKASSSLQLVFGIELMEVDPIGHLYIFATCLGLSYDGLLGDNQIMPKAGLLIIVLAIIAREGDCAPEEKIWEELSVLEVFEGREDSILGDPKKLLTQHFVQENYLEYRQVPGSDPACYEFLWGPRALVETSYVKVLHHMVKISGGPHISYPPLHEWVLREGEE

SEQ ID NO: 64 - protein, 317 aa, Homo sapiens
MSSEQKSQHCKPEEGVEAQEEALGLVGAQAPTTEEQEAAVSSSSPLVPGTLEEVPAAESAGPPQSPQGASALPTTISFTCWRQPNEGSSSQEEEGPSTSPDAESLFREALSNKVDELAHFLLRKYRAKELVTKAEMLERVIKNYKRCFPVIFGKASESLKMIFGIDVKEVDPTSNTYTLVTCLGLSYDGLLGNNQIFPKTGLLIIVLGTIAMEGDSASEEEIWEELGVMGVYDGREHTVYGEPRKLLTQDWVQENYLEYRQVPGSNPARYEFLWGPRALAETSYVKVLEHVVRVNARVRIAYPSLREAALLEEEEGV

SEQ ID NO: 65 - protein, 124 aa, Homo sapiens
MSLEQKSQHCKPEEGLDTQEEALGLVGVQAATTEEQEAVSSSSPLVPGTLGEVPAAGSPGPLKSPQGASAIPTAIDFTLWRQSIKGSSNQEEEGPSTSPDPESVFRAALSKKVADLIHFLLLKY

SEQ ID NO: 66 - protein, 314 aa, Homo sapiens
MPLEQRSQHCKPEEGLEARGEALGLVGAQAPATEEQEAASSSSTLVEVTLGEVPAAESPDPPQSPQGASSLPTTMNYPLWSQSYEDSSNQEEEGPSTFPDLESEFQAALSRKVAKLVHFLLLKYRAREPVTKAEMLGSVVGNWQYFFPVIFSKASDSLQLVFGIELMEVDPIGHVYIFATCLGLSYDGLLGDNQIMPKTGFLIIILAIIAKEGDCAPEEKIWEELSVLEVFEGREDSIFGDPKKLLTQYFVQENYLEYRQVPGSDPACYEFLWGPRALIETSYVKVLHHMVKISGGPRISYPLLHEWALREGEE

SEQ ID NO: 67 - protein, 318 aa, Homo sapiens
MLLGQKSQRYKAEEGLQAQGEAPGLMDVQIPTAEEQKAASSSSTLIMGTLEEVTDSGSPSPPQSPEGASSSLTVTDSTLWSQSDEGSSSNEEEGPSTSPDPAHLESLFREALDEKVAELVRFLLRKYQIKEPVTKAEMLESVIKNYKNHFPDIFSKASECMQVIFGIDVKEVDPAGHSYILVTCLGLSYDGLLGDDQSTPKTGLLIIVLGMILMEGSRAPEEAIWEALSVMGLYDGREHSVYWKLRKLLTQEWVQENYLEYRQAPGSDPVRYEFLWGPRALAETSYVKVLEHVVRVNARVRISYPSLHEEALGEEKGV

SEQ ID NO: 68 - protein, 315 aa, Homo sapiens
MSLEQRSPHCKPDEDLEAQGEDLGLMGAQEPTGEEEETTSSSDSKEEEVSAAGSSSPPQSPQGGASSSISVYYTLWSQFDEGSSSQEEEEPSSSVDPAQLEFMFQEALKLKVAELVHFLLHKYRVKEPVTKAEMLESVIKNYKRYFPVIFGKASEFMQVIFGTDVKEVDPAGHSYILVTALGLSCDSMLGDGHSMPKAALLIIVLGVILTKDNCAPEEVIWEALSVMGVYVGKEHMFYGEPRKLLTQDWVQENYLEYRQVPGSDPAHYEFLWGSKAHAETSYEKVINYLVMLNAREPICYPSLYEEVLGEEQEGV

SEQ ID NO: 69 - protein, 369 aa, Homo sapiens
MPRAPKRQRCMPEEDLQSQSETQGLEGAQAPLAVEEDASSSTSTSSSFPSSFPSSSSSSSSSCYPLIPSTPEEVSADDETPNPPQSAQIACSSPSVVASLPLDQSDEGSSSQKEESPSTLQVLPDSESLPRSEIDEKVTDLVQFLLFKYQMKEPITKAEILESVIRNYEDHFPLLFSEASECMLLVFGIDVKEVDPTGHSFVLVTSLGLTYDGMLSDVQSMPKTGILILILSIVFIEGYCTPEEVIWEALNMMGLYDGMEHLIYGEPRKLLTQDWVQENYLEYRQVPGSDPARYEFLWGPRAHAEIRKMSLLKFLAKVNGSDPRSFPLWYEEALKDEEERAQDRIATTDDTTAMASASSSATGSFSYPE

SEQ ID NO: 70 - protein, 429 aa, Homo sapiens
METQFRRGGLGCSPASIKRKKKREDSGDFGLQVSTMFSEDDFQSTERAPYGPQLQWSQDLPRVQVFREQANLEDRSPRRTQRITGGEQVLWGPITQIFPTVRPADLTRVIMPLEQRSQHCKPEEGLQAQEEDLGLVGAQALQAEEQEAAFFSSTLNVGTLEELPAAESPSPPQSPQEESFSPTAMDAIFGSLSDEGSGSQEKEGPSTSPDLIDPESFSQDILHDKIIDLVHLLLRKYRVKGLITKAEMLGSVIKNYEDYFPEIFREASVCMQLLFGIDVKEVDPTSHSYVLVTSLNLSYDGIQCNEQSMPKSGLLIIVLGVIFMEGNCIPEEVMWEVLSIMGVYAGREHFLFGEPKRLLTQNWVQEKYLVYRQVPGTDPACYEFLWGPRAHAETSKMKVLEYIANANGRDPTSYPSLYEDALREEGEGV

SEQ ID NO: 71 - protein, 314 aa, Homo sapiens
MPLEQRSQHCKPEEGLEAQGEALGLVGAQAPATEEQETASSSSTLVEVTLREVPAAESPSPPHSPQGASTLPTTINYTLWSQSDEGSSNEEQEGPSTFPDLETSFQVALSRKMAELVHFLLLKYRAREPFTKAEMLGSVIRNFQDFFPVIFSKASEYLQLVFGIEVVEVVRIGHLYILVTCLGLSYDGLLGDNQIVPKTGLLIIVLAIIAKEGDCAPEEKIWEELSVLEASDGREDSVFAHPRKLLTQDLVQENYLEYRQVPGSDPACYEFLWGPRALVETSYVKVLHHLLKISGGPHISYPPLHEWAFREGEE

SEQ ID NO: 72 - DNA, 21 bp, Artificial Sequence
  Synthetic Construct
  misc_feature 7, 8: positions 7 and 8 are linked by a hexa-(ethylene glycol) moiety
  misc_feature 14, 15: positions 14 and 15 are linked by a hexa-(ethylene glycol) moiety
tcgccggaacgttctcgccgg

SEQ ID NO: 73 - DNA, 50 bp, Artificial Sequence
  Synthetic Construct
  misc_feature 7, 8, 9, 10, 11, 12, 13, 14, 15, 16, 17, 18, 19, 20, 21,22, 23, 24, 25, 26, 27, 28, 29, 30, 31, 32, 33, 34, 35,36, 37, 38, 39, 40, 41, 42, 43, 44, 45, 46, 47, 48, 49,50: n = any nucleoside and up to 43 can be present or absent
aacgttnnnnnnnnnnnnnnnnnnnnnnnnnnnnnnnnnnnnnnnnnnnn

SEQ ID NO: 74 - protein, 25 aa, Artificial Sequence
  Synthetic Construct
GHGHSYTTAEELAGIGILTVILGVL